SEQUENCE LISTING (16 sequences)

SEQ ID NO: 1 - DNA, 2721 bp, Artificial Sequence
  Description of Artificial Sequence Synthetic polynucleotide
cctccgaagaagaagcggaaagtcgtggacctccggaccctgggttactctcagcagcagcaggagaagatcaagccgaaggtgcggtcgactgtggcccagcatcacgaggccctggtgggacacggcttcacccacgcccacattgtggccctgagccagcacccggcagcgctgggaaccgtggccgtgacctaccagcacatcattactgccctgcctgaagcgacccacgaggatatcgtgggcgtcggaaagcagtggtccggagccagagccttggaggctctgctgactgacgccggagagctgcggggcccgcccctgcaactggataccggccagctcgtgaaaatcgccaagagaggaggagtgaccgccatggaagccgtgcatgcatcccgcaatgcactgactggtgcacccctgaacctcactcctgaccaggtcgtcgctatcgcaagcaacatcggagggaaacaagctctcgagacagtgcagcgcctcctgccagtgctttgccaggaccacggcctgactccagaccaggtggtcgctattgcgtcgaacattggagggaagcaagcccttgaaaccgtgcagaggctgctcccggtgctgtgccaagaccatggactcaccccggaccaagtggtggctattgctagcaacattggcggtaagcaggcgctggagacagtccagcggctgctgccggtgttgtgccaagatcacggtcttaccccagaccaagtcgtggcgattgcctccaacggtggcggcaagcaagcactcgaaactgtccagagactgctccctgtgctctgtcaagaccacgggttgacccccgaccaagtggtggccatcgcctcccatgatggaggaaagcaggccctcgagactgtccagcgactgctccccgtgttgtgtcaggatcatggattgacgcccgatcaggtcgtggccattgcctcccacgacggtggaaagcaagcgctggaaactgtgcagcggttgctgccggtcctgtgccaggaccacggactgactccggaccaggtggtcgccatcgcatccaacattggtggcaagcaggctctcgaaaccgtccaacgcctgttgccggtgctgtgtcaggatcatggactgaccccggaccaagtggtggctatcgcctccaacaacgggggcaaacaggccctggaaaccgtgcaacgcctgctgcccgtcctctgccaggaccacggtctgacccctgaccaggtcgtcgcgatcgcgtcaaatggggggggcaaacaggctctggaaacggtgcagcggctccttccggtgttatgccaggaccacgggctgactcccgaccaggtggtggcgatcgcctcgaacaacggaggcaaacaagccctggagactgtgcagagactcctgcccgtgctgtgccaagaccatgggctcacccctgatcaggtggtggcaatcgcctcaaacatcggcgggaagcaggcactggaaactgtgcagagactcctgcccgtgctgtgccaagaccatgggctgaccccggaccaagtggtggctatcgcctcccacgacgggggcaaacaggccctggaatccatcgtcgctcagctgagccggcctgacccagcactcgccgccctgaccaatgaccatctggtcgccctggcctgcctgggaggcagacccgcgatggacgcggtcaagaagggtctgccgcacgcccctgagcttattcggagagtgaacaggcgcatcggtgaacgcacctcccatcgggtcgcaatctctagagtgggcggatcgtcccggcgggagtccatcaatccttggatcctgaccggcttcgccgacgccgaaggctccttcatcctggacatcaggaacaggaacaacgagtcaaacaggtaccgcacctcccttcggttccagattactctgcacaacaaggataagtccatcctcgagaacatccagtcaacctggaaagtgggcaagatcactaactcctcggaccgcgcagtgatgctccgggtgacccgcttcgaggacctgaaggtgatcattgaccacttcgagaagtaccctctcataacccagaagctgggagattacaagctgtttaagcaggcgttctccgtgatggagaacaaagaacaccttaaggagaatgggattaaggaactggtccgcattaaggccaagatgaactggggactgaacgacgagttgaaaaaggcatttcctgaaaacatctccaaggaacggccgctcatcaacaagaacattcccaatttcaagtggctggcggggttcactgccggggacggacacttcggagtgaacctgaagaaggtgaagggcaccgccaaggtgtacgtgggcctgcggttcgcgatcagccagcacatccgggataagaacctgatgaacagcctcatcacctacctgggatgcggaagcatccgggagaagaacaagtcagaattccgatggctggaatttgaagtgaccaagttctccgacatcaacgacaagatcatccccgtgttccaggagaacaccctcattggagtgaagctggaggacttcgaggactggtgcaaggtggccaagctcatcgaagagaagaagcacctgaccgaaagcggcctggatgagattaagaagattaagctcaacatgaacaagggaagatagtag

SEQ ID NO: 2 - RNA, 2957 nt, Artificial Sequence
  Description of Artificial Sequence Synthetic polynucleotide
ggggccgccaccaugggauccgccccuccgaagaagaagcggaaagucguggaccuccggacccuggguuacucucagcagcagcaggagaagaucaagccgaaggugcggucgacuguggcccagcaucacgaggcccuggugggacacggcuucacccacgcccacauuguggcccugagccagcacccggcagcgcugggaaccguggccgugaccuaccagcacaucauuacugcccugccugaagcgacccacgaggauaucgugggcgucggaaagcagugguccggagccagagccuuggaggcucugcugacugacgccggagagcugcggggcccgccccugcaacuggauaccggccagcucgugaaaaucgccaagagaggaggagugaccgccauggaagccgugcaugcaucccgcaaugcacugacuggugcaccccugaaccucacuccugaccaggucgucgcuaucgcaagcaacaucggagggaaacaagcucucgagacagugcagcgccuccugccagugcuuugccaggaccacggccugacuccagaccagguggucgcuauugcgucgaacauuggagggaagcaagcccuugaaaccgugcagaggcugcucccggugcugugccaagaccauggacucaccccggaccaagugguggcuauugcuagcaacauuggcgguaagcaggcgcuggagacaguccagcggcugcugccgguguugugccaagaucacggucuuaccccagaccaagucguggcgauugccuccaacgguggcggcaagcaagcacucgaaacuguccagagacugcucccugugcucugucaagaccacggguugacccccgaccaagugguggccaucgccucccaugauggaggaaagcaggcccucgagacuguccagcgacugcuccccguguugugucaggaucauggauugacgcccgaucaggucguggccauugccucccacgacgguggaaagcaagcgcuggaaacugugcagcgguugcugccgguccugugccaggaccacggacugacuccggaccagguggucgccaucgcauccaacauugguggcaagcaggcucucgaaaccguccaacgccuguugccggugcugugucaggaucauggacugaccccggaccaagugguggcuaucgccuccaacaacgggggcaaacaggcccuggaaaccgugcaacgccugcugcccguccucugccaggaccacggucugaccccugaccaggucgucgcgaucgcgucaaauggggggggcaaacaggcucuggaaacggugcagcggcuccuuccgguguuaugccaggaccacgggcugacucccgaccaggugguggcgaucgccucgaacaacggaggcaaacaagcccuggagacugugcagagacuccugcccgugcugugccaagaccaugggcucaccccugaucaggugguggcaaucgccucaaacaucggcgggaagcaggcacuggaaacugugcagagacuccugcccgugcugugccaagaccaugggcugaccccggaccaagugguggcuaucgccucccacgacgggggcaaacaggcccuggaauccaucgucgcucagcugagccggccugacccagcacucgccgcccugaccaaugaccaucuggucgcccuggccugccugggaggcagacccgcgauggacgcggucaagaagggucugccgcacgccccugagcuuauucggagagugaacaggcgcaucggugaacgcaccucccaucgggucgcaaucucuagagugggcggaucgucccggcgggaguccaucaauccuuggauccugaccggcuucgccgacgccgaaggcuccuucauccuggacaucaggaacaggaacaacgagucaaacagguaccgcaccucccuucgguuccagauuacucugcacaacaaggauaaguccauccucgagaacauccagucaaccuggaaagugggcaagaucacuaacuccucggaccgcgcagugaugcuccgggugacccgcuucgaggaccugaaggugaucauugaccacuucgagaaguacccucucauaacccagaagcugggagauuacaagcuguuuaagcaggcguucuccgugauggagaacaaagaacaccuuaaggagaaugggauuaaggaacugguccgcauuaaggccaagaugaacuggggacugaacgacgaguugaaaaaggcauuuccugaaaacaucuccaaggaacggccgcucaucaacaagaacauucccaauuucaaguggcuggcgggguucacugccggggacggacacuucggagugaaccugaagaaggugaagggcaccgccaagguguacgugggccugcgguucgcgaucagccagcacauccgggauaagaaccugaugaacagccucaucaccuaccugggaugcggaagcauccgggagaagaacaagucagaauuccgauggcuggaauuugaagugaccaaguucuccgacaucaacgacaagaucauccccguguuccaggagaacacccucauuggagugaagcuggaggacuucgaggacuggugcaagguggccaagcucaucgaagagaagaagcaccugaccgaaagcggccuggaugagauuaagaagauuaagcucaacaugaacaagggaagauaguagcgccguacggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 3 - RNA, 2721 nt, Artificial Sequence
  Description of Artificial Sequence Synthetic polynucleotide
ccuccgaagaagaagcggaaagucguggaccuccggacccuggguuacucucagcagcagcaggagaagaucaagccgaaggugcggucgacuguggcccagcaucacgaggcccuggugggacacggcuucacccacgcccacauuguggcccugagccagcacccggcagcgcugggaaccguggccgugaccuaccagcacaucauuacugcccugccugaagcgacccacgaggauaucgugggcgucggaaagcagugguccggagccagagccuuggaggcucugcugacugacgccggagagcugcggggcccgccccugcaacuggauaccggccagcucgugaaaaucgccaagagaggaggagugaccgccauggaagccgugcaugcaucccgcaaugcacugacuggugcaccccugaaccucacuccugaccaggucgucgcuaucgcaagcaacaucggagggaaacaagcucucgagacagugcagcgccuccugccagugcuuugccaggaccacggccugacuccagaccagguggucgcuauugcgucgaacauuggagggaagcaagcccuugaaaccgugcagaggcugcucccggugcugugccaagaccauggacucaccccggaccaagugguggcuauugcuagcaacauuggcgguaagcaggcgcuggagacaguccagcggcugcugccgguguugugccaagaucacggucuuaccccagaccaagucguggcgauugccuccaacgguggcggcaagcaagcacucgaaacuguccagagacugcucccugugcucugucaagaccacggguugacccccgaccaagugguggccaucgccucccaugauggaggaaagcaggcccucgagacuguccagcgacugcuccccguguugugucaggaucauggauugacgcccgaucaggucguggccauugccucccacgacgguggaaagcaagcgcuggaaacugugcagcgguugcugccgguccugugccaggaccacggacugacuccggaccagguggucgccaucgcauccaacauugguggcaagcaggcucucgaaaccguccaacgccuguugccggugcugugucaggaucauggacugaccccggaccaagugguggcuaucgccuccaacaacgggggcaaacaggcccuggaaaccgugcaacgccugcugcccguccucugccaggaccacggucugaccccugaccaggucgucgcgaucgcgucaaauggggggggcaaacaggcucuggaaacggugcagcggcuccuuccgguguuaugccaggaccacgggcugacucccgaccaggugguggcgaucgccucgaacaacggaggcaaacaagcccuggagacugugcagagacuccugcccgugcugugccaagaccaugggcucaccccugaucaggugguggcaaucgccucaaacaucggcgggaagcaggcacuggaaacugugcagagacuccugcccgugcugugccaagaccaugggcugaccccggaccaagugguggcuaucgccucccacgacgggggcaaacaggcccuggaauccaucgucgcucagcugagccggccugacccagcacucgccgcccugaccaaugaccaucuggucgcccuggccugccugggaggcagacccgcgauggacgcggucaagaagggucugccgcacgccccugagcuuauucggagagugaacaggcgcaucggugaacgcaccucccaucgggucgcaaucucuagagugggcggaucgucccggcgggaguccaucaauccuuggauccugaccggcuucgccgacgccgaaggcuccuucauccuggacaucaggaacaggaacaacgagucaaacagguaccgcaccucccuucgguuccagauuacucugcacaacaaggauaaguccauccucgagaacauccagucaaccuggaaagugggcaagaucacuaacuccucggaccgcgcagugaugcuccgggugacccgcuucgaggaccugaaggugaucauugaccacuucgagaaguacccucucauaacccagaagcugggagauuacaagcuguuuaagcaggcguucuccgugauggagaacaaagaacaccuuaaggagaaugggauuaaggaacugguccgcauuaaggccaagaugaacuggggacugaacgacgaguugaaaaaggcauuuccugaaaacaucuccaaggaacggccgcucaucaacaagaacauucccaauuucaaguggcuggcgggguucacugccggggacggacacuucggagugaaccugaagaaggugaagggcaccgccaagguguacgugggccugcgguucgcgaucagccagcacauccgggauaagaaccugaugaacagccucaucaccuaccugggaugcggaagcauccgggagaagaacaagucagaauuccgauggcuggaauuugaagugaccaaguucuccgacaucaacgacaagaucauccccguguuccaggagaacacccucauuggagugaagcuggaggacuucgaggacuggugcaagguggccaagcucaucgaagagaagaagcaccugaccgaaagcggccuggaugagauuaagaagauuaagcucaacaugaacaagggaagauaguag

SEQ ID NO: 4 - DNA, 2511 bp, Artificial Sequence
  Description of Artificial Sequence Synthetic polynucleotide
ccgccgaagaagaagcgcaaggtggtggatctgagaaccctgggatacagccagcagcagcaggagaagatcaagccgaaggtccggtctaccgtggcccagcaccatgaggcccttgtgggccacggcttcacacatgcacacatcgtcgccctgtcgcagcatcccgccgccctggggaccgtggccgtgacctatcaacacatcattaccgccctgccggaggccacccacgaggacatcgtgggtgtggggaagcagtggagcggagccagggcactcgaagccctcctcactgacgctggagaactgcgcggaccgcctctccagctggacaccggacagctggtgaaaatcgccaagcggggaggagtgaccgccatggaagccgtgcacgcctcgaggaacgcgctgactggcgcccctctgaacctgacccctgatcaggtcgtggctatcgcctcaaacaacgggggtaagcaggcgctggagacagtgcaacgacttctgccagtgctttgtcaggaccatggtctgacccccgaccaggtcgtcgccattgcatccaacaatggtggcaagcaggcactggagactgtccagaggctgctcccggtgctgtgccaggaccacgggctcaccccggaccaagtggtcgccatcgcctccaacggaggaggaaaacaagctctggagactgtgcaacgcctgctgcctgtgttgtgccaagaccacggactgacgcccgatcaggtggtggcgatcgcatcgaacaacggaggaaagcaagcgctggaaaccgtgcagcgcctcctgcccgtcctctgccaggatcacggcctgactccggaccaggtggtcgcgatcgccagcaataacggggggaagcaagccctcgagactgtgcagcggttgctgcccgtgctctgccaagatcatggccttaccccagaccaagtcgtggccattgcttccaacaacggtggcaaacaggcgctcgaaaccgtccagcggctgttgcccgtgctttgccaggatcacggactcacccctgatcaggtggtggcaattgcgtccaacaacggtggaaagcaggccctggaaacggtgcagcggctgcttccggtcctgtgtcaggatcatgggctgactcccgaccaggtcgtcgccattgcatcccacgatgggggtaaacaggccctcgaaacagtgcagagactcctgccagtcctgtgccaagaccacggacttaccccggatcaggtggtggccatagcctcgaacggcggcgggaaacaggctctggaaactgtgcaaagactcctcccggtgttgtgtcaagaccatggactgaccccagatcaggtggtggctattgcctctaacaacggcggcaagcaagcactcgaaagcatcgtggcccagttgtcacgccccgaccccgcactggctgccctgacgaatgaccatctggtggcgctggcctgcctgggagggaggccagcgatggatgcggtgaagaagggactgccccatgctccggagctgattcggagagtgaataggcgcatcggagagagaacttcacatcgggtggccatttctagagtgggcggcagctcccggcgcgagtccattaacccctggatcctgaccggctttgccgacgccgaagggtccttcggcctctcgatcctgaaccggaaccggggtaccgctcggtaccacaccagactgtccttcaccatcgtgctgcacaacaaggacaagagcatcctcgaaaacattcagtcaacgtggaaggtgggaattattactaacgacggcgacagatacgtgcgcctgtgcgtgacccggtttgaggacctgaaggtcattatcgaccacttcgagaagtaccccctcgtgactcagaagctgggagactacaagctgttcaagcaggcgttctcggtgatggaaaacaaggagcacctgaaggagaacggcatcaaggagctcgcccggatcaaggccaagatgaactggggcctgaatgatgaactcaagaaggcgttccctgaaaacatcggtaaagaacggcccctgatcaacaagaacatcccgaacttcaagtggcttgccggattcacctccggcgacggatccttcttcgtccggctgcgcaagtccaacgtgaacgcgagagtgcgggtgcaattggtctttgaaatctcacagcacatcagggacaagaatttgatgaactccctcatcacctacctgggttgcggacacatctacgaaggcaataagtcggagcggagctggctgcagtttcgcgtggaaaagttctccgacattaacgacaagatcatcccagtgttccaggaaaacactctgattggcgtgaagcttgaggatttcgaggactggtgcaaggtggccaagctgattgaagagaagaagcacctgaccgagtccggcctggacgaaatcaagaaaatcaagctgaacatgaacaagggacgg

SEQ ID NO: 5 - RNA, 2789 nt, Artificial Sequence
  Description of Artificial Sequence Synthetic polynucleotide
ggggccgccaccaugggaagcugccgguaccccuacgacgucccugacuacgccccgccgaagaagaagcgcaaggugguggaucugagaacccugggauacagccagcagcagcaggagaagaucaagccgaagguccggucuaccguggcccagcaccaugaggcccuugugggccacggcuucacacaugcacacaucgucgcccugucgcagcaucccgccgcccuggggaccguggccgugaccuaucaacacaucauuaccgcccugccggaggccacccacgaggacaucguggguguggggaagcaguggagcggagccagggcacucgaagcccuccucacugacgcuggagaacugcgcggaccgccucuccagcuggacaccggacagcuggugaaaaucgccaagcggggaggagugaccgccauggaagccgugcacgccucgaggaacgcgcugacuggcgccccucugaaccugaccccugaucaggucguggcuaucgccucaaacaacggggguaagcaggcgcuggagacagugcaacgacuucugccagugcuuugucaggaccauggucugacccccgaccaggucgucgccauugcauccaacaaugguggcaagcaggcacuggagacuguccagaggcugcucccggugcugugccaggaccacgggcucaccccggaccaaguggucgccaucgccuccaacggaggaggaaaacaagcucuggagacugugcaacgccugcugccuguguugugccaagaccacggacugacgcccgaucaggugguggcgaucgcaucgaacaacggaggaaagcaagcgcuggaaaccgugcagcgccuccugcccguccucugccaggaucacggccugacuccggaccagguggucgcgaucgccagcaauaacggggggaagcaagcccucgagacugugcagcgguugcugcccgugcucugccaagaucauggccuuaccccagaccaagucguggccauugcuuccaacaacgguggcaaacaggcgcucgaaaccguccagcggcuguugcccgugcuuugccaggaucacggacucaccccugaucaggugguggcaauugcguccaacaacgguggaaagcaggcccuggaaacggugcagcggcugcuuccgguccugugucaggaucaugggcugacucccgaccaggucgucgccauugcaucccacgauggggguaaacaggcccucgaaacagugcagagacuccugccaguccugugccaagaccacggacuuaccccggaucaggugguggccauagccucgaacggcggcgggaaacaggcucuggaaacugugcaaagacuccucccgguguugugucaagaccauggacugaccccagaucaggugguggcuauugccucuaacaacggcggcaagcaagcacucgaaagcaucguggcccaguugucacgccccgaccccgcacuggcugcccugacgaaugaccaucugguggcgcuggccugccugggagggaggccagcgauggaugcggugaagaagggacugccccaugcuccggagcugauucggagagugaauaggcgcaucggagagagaacuucacaucggguggccauuucuagagugggcggcagcucccggcgcgaguccauuaaccccuggauccugaccggcuuugccgacgccgaaggguccuucggccucucgauccugaaccggaaccgggguaccgcucgguaccacaccagacuguccuucaccaucgugcugcacaacaaggacaagagcauccucgaaaacauucagucaacguggaaggugggaauuauuacuaacgacggcgacagauacgugcgccugugcgugacccgguuugaggaccugaaggucauuaucgaccacuucgagaaguacccccucgugacucagaagcugggagacuacaagcuguucaagcaggcguucucggugauggaaaacaaggagcaccugaaggagaacggcaucaaggagcucgcccggaucaaggccaagaugaacuggggccugaaugaugaacucaagaaggcguucccugaaaacaucgguaaagaacggccccugaucaacaagaacaucccgaacuucaaguggcuugccggauucaccuccggcgacggauccuucuucguccggcugcgcaaguccaacgugaacgcgagagugcgggugcaauuggucuuugaaaucucacagcacaucagggacaagaauuugaugaacucccucaucaccuaccuggguugcggacacaucuacgaaggcaauaagucggagcggagcuggcugcaguuucgcguggaaaaguucuccgacauuaacgacaagaucaucccaguguuccaggaaaacacucugauuggcgugaagcuugaggauuucgaggacuggugcaagguggccaagcugauugaagagaagaagcaccugaccgaguccggccuggacgaaaucaagaaaaucaagcugaacaugaacaagggacggugauagcgcgcuagccguacggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 6 - RNA, 2511 nt, Artificial Sequence
  Description of Artificial Sequence Synthetic polynucleotide
ccgccgaagaagaagcgcaaggugguggaucugagaacccugggauacagccagcagcagcaggagaagaucaagccgaagguccggucuaccguggcccagcaccaugaggcccuugugggccacggcuucacacaugcacacaucgucgcccugucgcagcaucccgccgcccuggggaccguggccgugaccuaucaacacaucauuaccgcccugccggaggccacccacgaggacaucguggguguggggaagcaguggagcggagccagggcacucgaagcccuccucacugacgcuggagaacugcgcggaccgccucuccagcuggacaccggacagcuggugaaaaucgccaagcggggaggagugaccgccauggaagccgugcacgccucgaggaacgcgcugacuggcgccccucugaaccugaccccugaucaggucguggcuaucgccucaaacaacggggguaagcaggcgcuggagacagugcaacgacuucugccagugcuuugucaggaccauggucugacccccgaccaggucgucgccauugcauccaacaaugguggcaagcaggcacuggagacuguccagaggcugcucccggugcugugccaggaccacgggcucaccccggaccaaguggucgccaucgccuccaacggaggaggaaaacaagcucuggagacugugcaacgccugcugccuguguugugccaagaccacggacugacgcccgaucaggugguggcgaucgcaucgaacaacggaggaaagcaagcgcuggaaaccgugcagcgccuccugcccguccucugccaggaucacggccugacuccggaccagguggucgcgaucgccagcaauaacggggggaagcaagcccucgagacugugcagcgguugcugcccgugcucugccaagaucauggccuuaccccagaccaagucguggccauugcuuccaacaacgguggcaaacaggcgcucgaaaccguccagcggcuguugcccgugcuuugccaggaucacggacucaccccugaucaggugguggcaauugcguccaacaacgguggaaagcaggcccuggaaacggugcagcggcugcuuccgguccugugucaggaucaugggcugacucccgaccaggucgucgccauugcaucccacgauggggguaaacaggcccucgaaacagugcagagacuccugccaguccugugccaagaccacggacuuaccccggaucaggugguggccauagccucgaacggcggcgggaaacaggcucuggaaacugugcaaagacuccucccgguguugugucaagaccauggacugaccccagaucaggugguggcuauugccucuaacaacggcggcaagcaagcacucgaaagcaucguggcccaguugucacgccccgaccccgcacuggcugcccugacgaaugaccaucugguggcgcuggccugccugggagggaggccagcgauggaugcggugaagaagggacugccccaugcuccggagcugauucggagagugaauaggcgcaucggagagagaacuucacaucggguggccauuucuagagugggcggcagcucccggcgcgaguccauuaaccccuggauccugaccggcuuugccgacgccgaaggguccuucggccucucgauccugaaccggaaccgggguaccgcucgguaccacaccagacuguccuucaccaucgugcugcacaacaaggacaagagcauccucgaaaacauucagucaacguggaaggugggaauuauuacuaacgacggcgacagauacgugcgccugugcgugacccgguuugaggaccugaaggucauuaucgaccacuucgagaaguacccccucgugacucagaagcugggagacuacaagcuguucaagcaggcguucucggugauggaaaacaaggagcaccugaaggagaacggcaucaaggagcucgcccggaucaaggccaagaugaacuggggccugaaugaugaacucaagaaggcguucccugaaaacaucgguaaagaacggccccugaucaacaagaacaucccgaacuucaaguggcuugccggauucaccuccggcgacggauccuucuucguccggcugcgcaaguccaacgugaacgcgagagugcgggugcaauuggucuuugaaaucucacagcacaucagggacaagaauuugaugaacucccucaucaccuaccuggguugcggacacaucuacgaaggcaauaagucggagcggagcuggcugcaguuucgcguggaaaaguucuccgacauuaacgacaagaucaucccaguguuccaggaaaacacucugauuggcgugaagcuugaggauuucgaggacuggugcaagguggccaagcugauugaagagaagaagcaccugaccgaguccggccuggacgaaaucaagaaaaucaagcugaacaugaacaagggacgg

SEQ ID NO: 7 - DNA, 2925 bp, Artificial Sequence
  Description of Artificial Sequence Synthetic polynucleotide
cctcctaagaagaagcgcaaggtcgtggatctgcggaccctaggttactcccagcagcagcaggaaaagattaagccaaaggtccgctccaccgtggcgcagcaccacgaagcacttgtggggcacggattcacccacgcccacatcgtggccctgtcccaacatccggccgccctgggtactgtcgccgtgacttatcagcacattatcactgccctgcccgaggccacccacgaagatattgtgggagtcggaaagcagtggtcaggagcccgcgctctcgaggccctcctgaccgatgctggagagctgaggggtcctccgctccagctcgacactggacagctcgtgaaaatcgctaagaggggtggtgtgaccgccatggaagctgtgcacgccagccggaacgctctgactggagcccccctgaacctgactcctgatcaggtggttgctatagcttcgaacaatggcggtaagcaagcattggaaaccgtccaacggctgttgcccgtgctttgtcaagatcacggactgacccccgaccaggttgtggccattgcgtccaacaatggaggcaagcaagccctggagacagtgcagcggttgctgccggtgttgtgccaagatcatgggttaacgcctgaccaggtcgtcgcaatcgcctcaaataacgggggaaagcaggccctcgagactgtgcaacgcctcctgccggtcctctgccaagatcatggcctgacgccggatcaggtcgtggctatcgcaagccacgacggcgggaagcaagcactcgagactgtgcagagactgctcccggtcctgtgtcaagaccatggactcacgcccgatcaagtcgtcgcgattgccagcaacattggagggaaacaagctctcgaaaccgtgcaacgcttgctgcccgtcctgtgccaagaccacggactcacccccgatcaggtggtggcaatcgccagccacgatggggggaaacaggcattggaaactgtgcaaagactgctgccagtcttgtgccaggaccacgggctcacccctgatcaagtggtggcgatcgcaagccatgacggaggaaaacaagccttggagactgtccagaggctccttcccgtgttgtgtcaagaccatggcctcactccggaccaagtcgtggccatcgcctcgcacgacggaggcaaacaagcactggaaaccgtccagagattgctgcctgtcctgtgccaagaccacggtctgactcccgaccaagtggtcgccattgcatccaacggaggtggcaagcaggctttggaaaccgtacaacggctgctgcctgtgctctgccaggaccacgggctcaccccggatcaagttgtggctattgcctccaataacggcggaaaacaggccctggaaacggtccagcggctgcttccggtgctgtgccaggatcacgggctgacaccggaccaggtggtcgctatcgcctccaacaacggggggaagcaggctctcgaaaccgtgcagaggctgctccctgtgctttgccaggatcacggccttacccctgaccaggtcgtcgccatcgcttcgaacatcggtggtaagcaggcgctggaaactgtccaacgccttcttccagtgctgtgtcaggaccatgggcttaccccagaccaggtagtcgctatagcatcaaacggtggaggaaagcaggccttggaaacggtgcagcggctcctgccagtcctgtgccaagaccacggcctgaccccagatcaggtggtagccatcgccagcaataacggtggcaaacaggctctggagtccattgtggcccagctgtcgaggcctgacccggcgctggccgccctgaccaacgatcacctggtcgcgctggcctgtctcggaggcagacccgctatggacgcagtgaagaaaggcctgccccatgccccggaactgatccgcagggtgaatcgccggatcggcgaaagaaccagccatcgagtggccatctcccgcgtgggcggctcctccagacgcgagtccataaacccgtggatcctgaccgggttcgccgatgccgaaggttgcttcatgctgaatatttggaataagaaccgaactcgggccaaatactacaccgctctccgcttcgagatcgcgctccacaacaaggataagtccatactggagaacatccgatcgacctggaaagtcggcaagatccgcaacatcggggaccgggtggtgaagctggtcgtgggacggttcgaagatttgaaggtcattatcgaccatttcgagaagtaccctctgattacgcagaagctgggggactacaagctgttcaaacaggctttctccctgatggagaacaaggaacacttgaaggaaaacggcatcaaggaactggtccgcatcaaggccaagatgaactgggggctgaacgacgagctgaagaaggccttcccggaaaacattagcaaggagcgcccgctcatcaacaagaacatccccaacctgaagtggctggcgggattcacttccggcgacggttattttggagtggaactcatgaagagaacaaccggaacccacgtgaccgtgcgactccggttctccattacccagcatatccgggacaaaaacctgatgaacagcctgattacatacttgggatgtggccgcatttccgagcggaacaagtccgaatacagctggctggaattcgtggtcaccaagttcagcgacatcaacgacaagatcatcccagtgttccaggagaataccctgattggagtgaagctcgaggattttgaggactggtgcaaggtcgccaagctgatagaagaaaagaagcatctcaccgaatcaggcctggatgaaattaagaagatcaaactcaacatgaataagggacgcgtgttc

SEQ ID NO: 8 - RNA, 3227 nt, Artificial Sequence
  Description of Artificial Sequence Synthetic polynucleotide
ggggccgccaccauggggucaugucgguaccccuacgacguccccgacuacgcgccuccuaagaagaagcgcaaggucguggaucugcggacccuagguuacucccagcagcagcaggaaaagauuaagccaaagguccgcuccaccguggcgcagcaccacgaagcacuuguggggcacggauucacccacgcccacaucguggcccugucccaacauccggccgcccuggguacugucgccgugacuuaucagcacauuaucacugcccugcccgaggccacccacgaagauauugugggagucggaaagcaguggucaggagcccgcgcucucgaggcccuccugaccgaugcuggagagcugagggguccuccgcuccagcucgacacuggacagcucgugaaaaucgcuaagagggguggugugaccgccauggaagcugugcacgccagccggaacgcucugacuggagccccccugaaccugacuccugaucaggugguugcuauagcuucgaacaauggcgguaagcaagcauuggaaaccguccaacggcuguugcccgugcuuugucaagaucacggacugacccccgaccagguuguggccauugcguccaacaauggaggcaagcaagcccuggagacagugcagcgguugcugccgguguugugccaagaucauggguuaacgccugaccaggucgucgcaaucgccucaaauaacgggggaaagcaggcccucgagacugugcaacgccuccugccgguccucugccaagaucauggccugacgccggaucaggucguggcuaucgcaagccacgacggcgggaagcaagcacucgagacugugcagagacugcucccgguccugugucaagaccauggacucacgcccgaucaagucgucgcgauugccagcaacauuggagggaaacaagcucucgaaaccgugcaacgcuugcugcccguccugugccaagaccacggacucacccccgaucaggugguggcaaucgccagccacgauggggggaaacaggcauuggaaacugugcaaagacugcugccagucuugugccaggaccacgggcucaccccugaucaagugguggcgaucgcaagccaugacggaggaaaacaagccuuggagacuguccagaggcuccuucccguguugugucaagaccauggccucacuccggaccaagucguggccaucgccucgcacgacggaggcaaacaagcacuggaaaccguccagagauugcugccuguccugugccaagaccacggucugacucccgaccaaguggucgccauugcauccaacggagguggcaagcaggcuuuggaaaccguacaacggcugcugccugugcucugccaggaccacgggcucaccccggaucaaguuguggcuauugccuccaauaacggcggaaaacaggcccuggaaacgguccagcggcugcuuccggugcugugccaggaucacgggcugacaccggaccagguggucgcuaucgccuccaacaacggggggaagcaggcucucgaaaccgugcagaggcugcucccugugcuuugccaggaucacggccuuaccccugaccaggucgucgccaucgcuucgaacaucggugguaagcaggcgcuggaaacuguccaacgccuucuuccagugcugugucaggaccaugggcuuaccccagaccagguagucgcuauagcaucaaacgguggaggaaagcaggccuuggaaacggugcagcggcuccugccaguccugugccaagaccacggccugaccccagaucaggugguagccaucgccagcaauaacgguggcaaacaggcucuggaguccauuguggcccagcugucgaggccugacccggcgcuggccgcccugaccaacgaucaccuggucgcgcuggccugucucggaggcagacccgcuauggacgcagugaagaaaggccugccccaugccccggaacugauccgcagggugaaucgccggaucggcgaaagaaccagccaucgaguggccaucucccgcgugggcggcuccuccagacgcgaguccauaaacccguggauccugaccggguucgccgaugccgaagguugcuucaugcugaauauuuggaauaagaaccgaacucgggccaaauacuacaccgcucuccgcuucgagaucgcgcuccacaacaaggauaaguccauacuggagaacauccgaucgaccuggaaagucggcaagauccgcaacaucggggaccggguggugaagcuggucgugggacgguucgaagauuugaaggucauuaucgaccauuucgagaaguacccucugauuacgcagaagcugggggacuacaagcuguucaaacaggcuuucucccugauggagaacaaggaacacuugaaggaaaacggcaucaaggaacugguccgcaucaaggccaagaugaacugggggcugaacgacgagcugaagaaggccuucccggaaaacauuagcaaggagcgcccgcucaucaacaagaacauccccaaccugaaguggcuggcgggauucacuuccggcgacgguuauuuuggaguggaacucaugaagagaacaaccggaacccacgugaccgugcgacuccgguucuccauuacccagcauauccgggacaaaaaccugaugaacagccugauuacauacuugggauguggccgcauuuccgagcggaacaaguccgaauacagcuggcuggaauucguggucaccaaguucagcgacaucaacgacaagaucaucccaguguuccaggagaauacccugauuggagugaagcucgaggauuuugaggacuggugcaaggucgccaagcugauagaagaaaagaagcaucucaccgaaucaggccuggaugaaauuaagaagaucaaacucaacaugaauaagggacgcguguucagcggacgcuagugacgcgcuagccauuuaaauguuaaucguacggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 9 - RNA, 2925 nt, Artificial Sequence
  Description of Artificial Sequence Synthetic polynucleotide
ccuccuaagaagaagcgcaaggucguggaucugcggacccuagguuacucccagcagcagcaggaaaagauuaagccaaagguccgcuccaccguggcgcagcaccacgaagcacuuguggggcacggauucacccacgcccacaucguggcccugucccaacauccggccgcccuggguacugucgccgugacuuaucagcacauuaucacugcccugcccgaggccacccacgaagauauugugggagucggaaagcaguggucaggagcccgcgcucucgaggcccuccugaccgaugcuggagagcugagggguccuccgcuccagcucgacacuggacagcucgugaaaaucgcuaagagggguggugugaccgccauggaagcugugcacgccagccggaacgcucugacuggagccccccugaaccugacuccugaucaggugguugcuauagcuucgaacaauggcgguaagcaagcauuggaaaccguccaacggcuguugcccgugcuuugucaagaucacggacugacccccgaccagguuguggccauugcguccaacaauggaggcaagcaagcccuggagacagugcagcgguugcugccgguguugugccaagaucauggguuaacgccugaccaggucgucgcaaucgccucaaauaacgggggaaagcaggcccucgagacugugcaacgccuccugccgguccucugccaagaucauggccugacgccggaucaggucguggcuaucgcaagccacgacggcgggaagcaagcacucgagacugugcagagacugcucccgguccugugucaagaccauggacucacgcccgaucaagucgucgcgauugccagcaacauuggagggaaacaagcucucgaaaccgugcaacgcuugcugcccguccugugccaagaccacggacucacccccgaucaggugguggcaaucgccagccacgauggggggaaacaggcauuggaaacugugcaaagacugcugccagucuugugccaggaccacgggcucaccccugaucaagugguggcgaucgcaagccaugacggaggaaaacaagccuuggagacuguccagaggcuccuucccguguugugucaagaccauggccucacuccggaccaagucguggccaucgccucgcacgacggaggcaaacaagcacuggaaaccguccagagauugcugccuguccugugccaagaccacggucugacucccgaccaaguggucgccauugcauccaacggagguggcaagcaggcuuuggaaaccguacaacggcugcugccugugcucugccaggaccacgggcucaccccggaucaaguuguggcuauugccuccaauaacggcggaaaacaggcccuggaaacgguccagcggcugcuuccggugcugugccaggaucacgggcugacaccggaccagguggucgcuaucgccuccaacaacggggggaagcaggcucucgaaaccgugcagaggcugcucccugugcuuugccaggaucacggccuuaccccugaccaggucgucgccaucgcuucgaacaucggugguaagcaggcgcuggaaacuguccaacgccuucuuccagugcugugucaggaccaugggcuuaccccagaccagguagucgcuauagcaucaaacgguggaggaaagcaggccuuggaaacggugcagcggcuccugccaguccugugccaagaccacggccugaccccagaucaggugguagccaucgccagcaauaacgguggcaaacaggcucuggaguccauuguggcccagcugucgaggccugacccggcgcuggccgcccugaccaacgaucaccuggucgcgcuggccugucucggaggcagacccgcuauggacgcagugaagaaaggccugccccaugccccggaacugauccgcagggugaaucgccggaucggcgaaagaaccagccaucgaguggccaucucccgcgugggcggcuccuccagacgcgaguccauaaacccguggauccugaccggguucgccgaugccgaagguugcuucaugcugaauauuuggaauaagaaccgaacucgggccaaauacuacaccgcucuccgcuucgagaucgcgcuccacaacaaggauaaguccauacuggagaacauccgaucgaccuggaaagucggcaagauccgcaacaucggggaccggguggugaagcuggucgugggacgguucgaagauuugaaggucauuaucgaccauuucgagaaguacccucugauuacgcagaagcugggggacuacaagcuguucaaacaggcuuucucccugauggagaacaaggaacacuugaaggaaaacggcaucaaggaacugguccgcaucaaggccaagaugaacugggggcugaacgacgagcugaagaaggccuucccggaaaacauuagcaaggagcgcccgcucaucaacaagaacauccccaaccugaaguggcuggcgggauucacuuccggcgacgguuauuuuggaguggaacucaugaagagaacaaccggaacccacgugaccgugcgacuccgguucuccauuacccagcauauccgggacaaaaaccugaugaacagccugauuacauacuugggauguggccgcauuuccgagcggaacaaguccgaauacagcuggcuggaauucguggucaccaaguucagcgacaucaacgacaagaucaucccaguguuccaggagaauacccugauuggagugaagcucgaggauuuugaggacuggugcaaggucgccaagcugauagaagaaaagaagcaucucaccgaaucaggccuggaugaaauuaagaagaucaaacucaacaugaauaagggacgcguguuc

SEQ ID NO: 10 - DNA, 711 bp, Artificial Sequence
  Description of Artificial Sequence Synthetic polynucleotide
atgtcagaaccacctcgcgctgagactttcgtgttccttgaccttgaagccacaggactgcccaacatggacccggaaatcgccgagatcagcctcttcgcggtgcatcggtcatccctggagaaccccgaacgggacgattccgggtcactggtgttgcctcgcgtgctggataagctgactctctgcatgtgccctgaaaggccgttcaccgccaaagcgtcggaaatcaccggtctgtcgtccgaatccctgatgcattgcggaaaggccgggttcaatggtgccgtcgtcagaaccctgcagggattcctgtcacggcaggaaggtcctatctgcctggtggcacacaacggcttcgactacgactttcccctcctgtgtaccgagctgcagagactcggagcccatctgccacaggatactgtgtgcctcgacactctgcctgcgctcagaggacttgatcgggcacactcccatggaaccagggctcagggaagaaagtcctacagcttggcctcgctgttccaccggtacttccaggcagaaccatcggccgcacattcagctgagggcgatgtgcacaccctgctgctgatcttccttcaccgcgcacctgaactgctggcttgggcagacgaacaagctagatcctgggcgcacattgagcctatgtacgtgcctccggatggacctagcctcgaggcctag

SEQ ID NO: 11 - RNA, 964 nt, Artificial Sequence
  Description of Artificial Sequence Synthetic polynucleotide
ggggccgccaccaugucagaaccaccucgcgcugagacuuucguguuccuugaccuugaagccacaggacugcccaacauggacccggaaaucgccgagaucagccucuucgcggugcaucggucaucccuggagaaccccgaacgggacgauuccgggucacugguguugccucgcgugcuggauaagcugacucucugcaugugcccugaaaggccguucaccgccaaagcgucggaaaucaccggucugucguccgaaucccugaugcauugcggaaaggccggguucaauggugccgucgucagaacccugcagggauuccugucacggcaggaagguccuaucugccugguggcacacaacggcuucgacuacgacuuuccccuccuguguaccgagcugcagagacucggagcccaucugccacaggauacugugugccucgacacucugccugcgcucagaggacuugaucgggcacacucccauggaaccagggcucagggaagaaaguccuacagcuuggccucgcuguuccaccgguacuuccaggcagaaccaucggccgcacauucagcugagggcgaugugcacacccugcugcugaucuuccuucaccgcgcaccugaacugcuggcuugggcagacgaacaagcuagauccugggcgcacauugagccuauguacgugccuccggauggaccuagccucgaggccuaguuaauuaacgagcaucuuaccgccauuuauaccguacggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 12 - RNA, 711 nt, Artificial Sequence
  Description of Artificial Sequence Synthetic polynucleotide
augucagaaccaccucgcgcugagacuuucguguuccuugaccuugaagccacaggacugcccaacauggacccggaaaucgccgagaucagccucuucgcggugcaucggucaucccuggagaaccccgaacgggacgauuccgggucacugguguugccucgcgugcuggauaagcugacucucugcaugugcccugaaaggccguucaccgccaaagcgucggaaaucaccggucugucguccgaaucccugaugcauugcggaaaggccggguucaauggugccgucgucagaacccugcagggauuccugucacggcaggaagguccuaucugccugguggcacacaacggcuucgacuacgacuuuccccuccuguguaccgagcugcagagacucggagcccaucugccacaggauacugugugccucgacacucugccugcgcucagaggacuugaucgggcacacucccauggaaccagggcucagggaagaaaguccuacagcuuggccucgcuguuccaccgguacuuccaggcagaaccaucggccgcacauucagcugagggcgaugugcacacccugcugcugaucuuccuucaccgcgcaccugaacugcuggcuugggcagacgaacaagcuagauccugggcgcacauugagccuauguacgugccuccggauggaccuagccucgaggccuag

SEQ ID NO: 13 - DNA, 300 bp, Artificial Sequence
  Description of Artificial Sequence Synthetic polynucleotide
  misc_feature (1)..(300): This sequence may encompass 5-300 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 14 - protein, 9 aa, Unknown
  Description of Unknown “LAGLIDADG” family peptide motif sequence
LAGLIDADG

SEQ ID NO: 15 - DNA, 25 bp, Artificial Sequence
  Description of Artificial Sequence Synthetic oligonucleotide
ttttttttttttttttttttttttt

SEQ ID NO: 16 - DNA, 23 bp, Artificial Sequence
  Description of Artificial Sequence Synthetic oligonucleotide
ttttttttttttttttttttttt

(19) United States
(12) Patent Application Publication
(10) Pub. No.: US 2024/0218351 A1
(43) Pub. Date: Jul. 4, 2024

(54) SINGLE STRANDED RNA PURIFICATION METHODS

(71) Applicant: 2SEVENTY BIO, INC., Cambridge, MA (US)

(72) Inventors: Michael T. Certo, Bozeman, MT (US); Aaron David Edwards, Boston, MA (US); Joseph Louis Barberio, Marborough, MA (US)

(21) Appl. No.: 18/569,665

(22) PCT Filed: Jun. 14, 2022
(86) PCT No.: PCT/US2022/033346

§ 371 Date: Dec. 13, 2023

Related U.S. Application Data
(60) Provisional application No. 63/210,101, filed on Jun. 14, 2021.

(51) Int. Cl. C12N 15/10; A61K 31/7105; C12N 15/11

(52) U.S. Cl. CPC C12N 15/1006; A61K 31/7105; C12N 15/11

(57) ABSTRACT

The present disclosure relates to improved therapeutic RNA compositions. More particularly, the disclosure relates to improved methods for purifying therapeutic RNA and related therapeutic RNA preparations.

### CROSS REFERENCE TO RELATED APPLICATIONS

This application claims the benefit under 35 U.S.C. § 119(e) of U.S. Provisional Application No. 63/210,101, filed Jun. 14, 2021, which is incorporated by reference herein in its entirety.

### STATEMENT REGARDING SEQUENCE LISTING

The Sequence Listing associated with this application is provided in text format in lieu of a paper copy and is hereby incorporated by reference into the specification. The name of the text file containing the Sequence Listing is BLUE-136_PC_SL.txt. The text file is 39,804 bytes in size, created on Jun. 9, 2022, and is being submitted electronically via EFS-Web, concurrent with the filing of the specification.

### BACKGROUND

### Technical Field

The present disclosure relates to improved RNA compositions. More particularly, the disclosure relates to improved methods for purifying therapeutic RNA and related therapeutic RNA preparations.

### Description of the Related Art

Over the past 30-40 years there has been a slow emergence of RNA-based technologies, which have garnered increased attention in recent years as new methods for RNA delivery have been developed and proven effective in vivo. For example, RNAi (e.g., siRNA, shRNA, or miRNA), ribozymes, aptamers, and the related techniques have been used to reduce expression or modulate the activity of disease-associated proteins. In other cases, RNA has been used to express proteins either in vitro, ex vivo, or in vivo for therapeutic purposes. However, double stranded RNA (dsRNA) can be toxic to cells in certain situations. There remains a need to develop improved methods for specifically removing dsRNA from RNA preparations, particularly for therapeutic use in vivo.

### BRIEF SUMMARY

The present disclosure generally relates, in part, to RNA purification methods/processes comprising a dsRNA removal step. In some embodiments, the methods/processes comprise one or more oligo dT purification steps and a dsRNA removal step.

In one aspect, an RNA purification process is provided, comprising: contacting an RNA sample comprising single-stranded RNA and double-stranded RNA (dsRNA) with an antibody or antigen binding fragment thereof that binds dsRNA, thus forming dsRNA:antibody complexes; removing the dsRNA:antibody complexes from the sample; and purifying the single-stranded RNA.

In another aspect, an RNA purification process is provided, comprising: contacting an RNA sample comprising single-stranded RNA and double-stranded RNA (dsRNA) with an antibody or antigen binding fragment thereof that binds dsRNA, thus forming dsRNA:antibody complexes; removing the dsRNA:antibody complexes from the sample; and purifying the single-stranded RNA; thereby producing therapeutic RNA.

In another aspect, an RNA purification process is provided, comprising: contacting an RNA sample comprising single-stranded RNA and double-stranded RNA (dsRNA) encoding a nuclease with an antibody or antigen binding fragment thereof that binds dsRNA, thus forming dsRNA:antibody complexes; removing the dsRNA:antibody complexes from the sample; and purifying the single-stranded RNA; wherein the editing rate of the nuclease is increased compared to the editing rate of a nuclease encoded by an RNA that is not contacted with an antibody that binds dsRNA.

In another aspect, an RNA purification process is provided, comprising: contacting an RNA sample comprising single-stranded RNA and double-stranded RNA (dsRNA) with an antibody or antigen binding fragment thereof that binds dsRNA, thus forming dsRNA:antibody complexes; removing the dsRNA:antibody complexes from the sample; and purifying the single-stranded RNA; wherein the immunogenicity and/or toxicity of the RNA when administered to a cell or subject is less than the immunogenicity and/or toxicity of an RNA administered to a cell or subject when the RNA has not been contacted with an antibody that binds dsRNA. In some embodiments, the subject is human.

In various embodiments, the single-stranded RNA is single-stranded circular RNA, single-stranded mRNA, or single-stranded non-coding RNA.

In various embodiments, the single-stranded RNA is polyadenylated and/or the process comprises a polyadenylation step prior to contacting the sample with an antibody or antigen binding fragment thereof that binds dsRNA.

In various embodiments, the process comprises contacting the polyadenylated RNA sample with a first oligonucleotide dT (oligo dT) probe that binds polyadenylated RNA and removing unbound RNA from the sample prior to contacting the sample with an antibody or antigen binding fragment thereof that binds dsRNA.

In various embodiments, the process further comprises contacting the polyadenylated RNA with a second oligonucleotide dT probe after contacting with the antibody or antigen binding fragment thereof that binds dsRNA.

In various embodiments, the RNA sample is obtained de novo through chemical synthesis. In some embodiments, the RNA sample is obtained from an in vitro transcription reaction.

In various embodiments, cytotoxicity, as measured by impedance, of the purified RNA when administered to a cell is less than the cytotoxicity of RNA administered to a cell, when the RNA has not been contacted with an antibody that binds dsRNA and/or a second oligo dT.

In various embodiments, the first and/or second oligonucleotide dT probe is bound to a surface. In some embodiments, the first and/or second oligonucleotide dT probe is covalently linked to the surface.

In various embodiments, the RNA in the sample is capped and/or the process comprises capping the RNA in the sample. In some embodiments, the RNA is obtained from an in vitro transcription reaction and is co-transcriptionally capped. In some embodiments, the cap is a cap0 or cap1. In some embodiments, the cap is an ARCA cap or modified ARCA cap. In some embodiments, the RNA in the sample is capped at its 5′ end using a capping enzyme, guanosine triphosphate, and S-adenosyl-L-methionine. In particular embodiments, the capping enzyme is Vaccinia guanylyltransferase. In some embodiments, the capping comprises guanosine triphosphate. In some embodiments, the capping comprises, S-adenosyl-L-methionine. In some embodiments, the capping comprises a 2′-O-Methyltransferase.

In various embodiments, the antibody or antigen binding fragment thereof that binds dsRNA is selected from the group consisting of: a Camel Ig, a Llama Ig, an Alpaca Ig, Ig NAR, a Fab′ fragment, a F(ab′)2 fragment, a bispecific Fab dimer (Fab2), a trispecific Fab trimer (Fab3), an Fv, an single chain Fv protein (“scFv”), a bis-scFv, (scFv)2, a minibody, a diabody, a triabody, a tetrabody, a disulfide stabilized Fv protein (“dsFv”), and a single-domain antibody (sdAb, a camelid VHH, Nanobody). In some embodiments, the antibody or antigen binding fragment thereof that binds dsRNA is a monoclonal antibody. In some embodiments, the antibody is selected from the group consisting of: J2, J5, K1, K2, 1D3, CABT-B212, and 9D5. In particular embodiments, the antibody is J2.

In various embodiments, the sample is contacted with at least about 1.5 mol %, at about least 2 mol %, at least about 2.5 mol %, at least about 3 mol %, at least about 3.5 mol %, at least about 4 mol %, at least about 4.5 mol %, at least about 5 mol %, at least about 5.5 mol %, at least about 6 mol %, at least about 6.5 mol %, at least about 7 mol %, at least about 7.5 mol %, at least about 15 mol %, at least about 30 mol %, or at least about 60 mol % antibody, compared to total moles of RNA within the sample. In some embodiments, the sample is contacted with at least about 1.5 mol %, at least about 7.5 mol %, at least about 15 mol %, at least about 30 mol %, or at least about 60 mol % antibody, compared to total moles of RNA within the sample. In some embodiments, the sample is contacted with at least about 7.5 mol % antibody, compared to total moles of RNA within the sample. In some embodiments, the sample is contacted with about 1.5 mol %, about 2 mol %, about 2.5 mol %, about 3 mol %, about 3.5 mol %, about 4 mol %, about 4.5 mol %, about 5 mol %, about 5.5 mol %, about 6 mol %, about 6.5 mol %, about 7 mol %, about 7.5 mol %, about 15 mol %, about 30 mol %, or about 60 mol % antibody, compared to total moles of RNA within the sample. In some embodiments, the sample is contacted with about 7.5 mol % antibody, compared to total moles of RNA within the sample. In some embodiments, the sample is contacted with about 1.5 mol % to about 60 mol % antibody, compared to total moles of RNA within the sample. In some embodiments, the sample is contacted with about 2 mol % to about 60 mol % antibody, compared to total moles of RNA within the sample. In some embodiments, the sample is contacted with about 2.5 mol % to about 60 mol % antibody, compared to total moles of RNA within the sample. In some embodiments, the sample is contacted with about 3 mol % to about 60 mol % antibody, compared to total moles of RNA within the sample. In some embodiments, the sample is contacted with about 3.5 mol % to about 60 mol % antibody, compared to total moles of RNA within the sample. In some embodiments, the sample is contacted with about 4 mol % to about 60 mol % antibody, compared to total moles of RNA within the sample. In some embodiments, the sample is contacted with about 4.5 mol % to about 60 mol % antibody, compared to total moles of RNA within the sample. In some embodiments, the sample is contacted with about 5 mol % to about 60 mol % antibody, compared to total moles of RNA within the sample. In some embodiments, the sample is contacted with about 5.5 mol % to about 60 mol % antibody, compared to total moles of RNA within the sample. In some embodiments, the sample is contacted with about 6 mol % to about 60 mol % antibody, compared to total moles of RNA within the sample. In some embodiments, the sample is contacted with about 6.5 mol % to about 60 mol % antibody, compared to total moles of RNA within the sample. In some embodiments, the sample is contacted with about 7 mol % to about 60 mol % antibody, compared to total moles of RNA within the sample. In some embodiments, the sample is contacted with about 7.5 mol % to about 60 mol % antibody, compared to total moles of RNA within the sample. In some embodiments, the sample is contacted with about 15 mol % to about 60 mol % antibody, compared to total moles of RNA within the sample. In some embodiments, the sample is contacted with about 30 mol % to about 60 mol % antibody, compared to total moles of RNA within the sample. In some embodiments, the sample is contacted with about 1.5 mol % to about 30 mol % antibody, compared to total moles of RNA within the sample. In some embodiments, the sample is contacted with about 1.5 mol % to about 15 mol % antibody, compared to total moles of RNA within the sample. In some embodiments, the sample is contacted with about 1.5 mol % to about 7.5 mol % antibody, compared to total moles of RNA within the sample. In some embodiments, the sample is contacted with about 1.5 mol % to about 7 mol % antibody, compared to total moles of RNA within the sample. In some embodiments, the sample is contacted with about 1.5 mol % to about 6.5 mol % antibody, compared to total moles of RNA within the sample. In some embodiments, the sample is contacted with about 1.5 mol % to about 6 mol % antibody, compared to total moles of RNA within the sample. In some embodiments, the sample is contacted with about 1.5 mol % to about 5.5 mol % antibody, compared to total moles of RNA within the sample. In some embodiments, the sample is contacted with about 1.5 mol % to about 5 mol % antibody, compared to total moles of RNA within the sample. In some embodiments, the sample is contacted with about 1.5 mol % to about 4.5 mol % antibody, compared to total moles of RNA within the sample. In some embodiments, the sample is contacted with about 1.5 mol % to about 4 mol % antibody, compared to total moles of RNA within the sample. In some embodiments, the sample is contacted with about 1.5 mol % to about 3.5 mol % antibody, compared to total moles of RNA within the sample. In some embodiments, the sample is contacted with about 1.5 mol % to about 3 mol % antibody, compared to total moles of RNA within the sample. In some embodiments, the sample is contacted with about 1.5 mol % to about 2.5 mol % antibody, compared to total moles of RNA within the sample. In some embodiments, the sample is contacted with about 1.5 mol % to about 2 mol % antibody, compared to total moles of RNA within the sample.

In various embodiments, the dsRNA:antibody complex is separated from the single-stranded RNA by antibody-based affinity chromatography. In some embodiments, the antibody-based affinity chromatography comprises a 1 ml column. In some embodiments, the antibody-based affinity chromatography comprises a 5 ml column. In some embodiments, the antibody-based affinity chromatography comprises a 10 ml column.

In various embodiments, the process comprises a plasmid digestion step prior to an IVT step.

In various embodiments, the process further comprises a step of treating the sample with a DNase to remove residual plasmid DNA template. In some embodiments, the DNase treatment step occurs after an IVT step and/or after a capping step.

In various embodiments, the process further comprises one or more ultrafiltration/diafiltration (UF/DF) step(s). In some embodiments, the UF/DF step is after a plasmid digestion step, an in vitro transcription step, a cap reaction step, or an affinity chromatography step (e.g., dT or J2).

In various embodiments, the process further comprises a final sterile filtration step. In some embodiments, the final sterile filtration step comprises filtration through a 0.22 μm filter.

In various embodiments, the nuclease is an endonuclease or exonuclease. In some embodiments, the nuclease is a homing endonuclease, megaTAL, CRISPR-associated nuclease, zinc finger nuclease, transcription activator-like effector nuclease (TALEN). In some embodiments, the CRISPR-associated nuclease is Cas9 or a variant thereof.

In various embodiments, Aspartate Aminotransferase enzyme (AST) levels in a subject administered the purified RNA are less than AST levels in a subject administered purified RNA not contacted with an antibody that binds dsRNA and/or a second oligo dT.

In various embodiments, IL-6 levels in a subject administered the purified RNA are less than IL-6 levels in a subject administered purified RNA not contacted with an antibody that binds dsRNA and/or a second oligo dT.

In various embodiments, MCP-1 levels in a subject administered the purified RNA are less than MCP-1 levels in a subject administered purified RNA not contacted with an antibody that binds dsRNA and/or a second oligo dT.

### BRIEF DESCRIPTION OF SEVERAL VIEWS OF THE DRAWINGS

FIGS. 1A-1F show different unit operations for illustrative RNA purification methods.

FIG. 2A shows a dsRNA dot blot analysis of purified mRNA using different chromatography column volumes to remove excess anti-dsRNA antibodies.

FIG. 2B shows % dsRNA content in samples of purified mRNA using different chromatography column volumes to remove excess anti-dsRNA antibodies.

FIG. 2C shows a dot blot analysis of purified mRNA using fluorescently-labeled secondary to directly blot for residual anti-dsRNA antibodies.

FIGS. 3A and 3B show % dsRNA content in samples of purified mRNA using different amounts of anti-dsRNA antibodies.

FIGS. 3C and 3D show in vitro cytotoxicity of purified mRNA using different amounts of anti-dsRNA antibodies

FIG. 4A shows % full length mRNA in samples purified by different methods.

FIG. 4B shows % dsRNA content in samples purified by different methods.

FIG. 4C shows in vitro cytotoxicity of mRNA samples purified by different methods.

FIG. 4D shows % INDEL fold change after in vivo editing by PCSK9 megaTAL and Trex2 encoded by mRNA purified by different methods.

FIG. 4E shows in vivo toxicity of PCSK9 megaTAL and Trex2 mRNA purified by different methods.

FIG. 4F shows the degree of immunogenicity (cytokine/chemokine release) induced by PCSK9 megaTAL and Trex2 mRNA purified by different methods.

FIG. 5A shows % full length mRNA in samples purified by different methods.

FIG. 5B shows % dsRNA content in samples purified by different methods.

FIG. 5C shows in vitro cytotoxicity of mRNA samples purified by different methods.

FIG. 5D shows % INDEL after ex vivo editing by PD-1 megaTAL encoded by mRNA purified by different methods.

FIG. 5E shows % PD-1 surface expression after ex vivo editing by PD-1 megaTAL encoded by mRNA purified by different methods.

FIG. 6A shows % dsRNA content among different methods of RNA purification.

FIG. 6B shows the correlation between cellular cytotoxicity of RNA preparations and % dsRNA content.

### BRIEF DESCRIPTION OF THE SEQUENCE IDENTIFIERS

SEQ ID NO: 1 is a TCRα megaTAL DNA sequence.

SEQ ID NO: 2 is a TCRα megaTAL RNA sequence.

SEQ ID NO: 3 is a TCRα megaTAL RNA sequence.

SEQ ID NO: 4 is a PD1 megaTAL DNA sequence.

SEQ ID NO: 5 is a PD1 megaTAL RNA sequence.

SEQ ID NO: 6 is a PD1 megaTAL RNA sequence.

SEQ ID NO: 7 is a PCSK9 megaTAL DNA sequence.

SEQ ID NO: 8 is a PCSK9 megaTAL RNA sequence.

SEQ ID NO: 9 is a PCSK9 megaTAL RNA sequence.

SEQ ID NO: 10 is a Trex2 DNA sequence.

SEQ ID NO: 11 is a Trex2 RNA sequence.

SEQ ID NO: 12 is a Trex2 RNA sequence.

In the foregoing sequences, X, if present, refers to any amino acid or the absence of an amino acid.

### DETAILED DESCRIPTION

### A. Overview

The present disclosure generally relates to, in part, improved methods of RNA purification and preparation of RNA compositions. More particularly, the disclosure relates to improved methods for separating double stranded RNA (dsRNA) from therapeutic single-stranded therapeutic RNA and related therapeutic single stranded RNA compositions. The RNA preparations may be for use in vitro, ex vivo, or in vivo. Without wishing to be bound by any particular theory, the inventors have discovered that RNA purification using an anti-dsRNA antibody (e.g., antibody-based affinity chromatography) is surprisingly effective at purifying single-stranded RNA and reducing the immunogenicity and cellular toxicity of the RNA delivered to cells ex vivo and in vivo. In particular embodiments, the RNA purification method comprises one or more oligo dT purification steps and an anti-dsRNA antibody-based removal step.

Accordingly, the problem of RNA immunogenicity and toxicity is solved by utilizing anti-dsRNA antibody removal and/or oligo dT purification, as described further herein. RNA purified using the compositions and methods contemplated in particular embodiments, are suitable for use in in vitro, ex vivo, or in vivo applications.

In one aspect, an RNA purification process is provided, comprising: contacting an RNA sample comprising single-stranded RNA and double-stranded RNA (dsRNA) with an antibody or antigen binding fragment thereof that binds dsRNA, thus forming dsRNA:antibody complexes; removing the dsRNA:antibody complexes from the sample; and purifying the RNA. In various embodiments, the single-stranded RNA is single-stranded circular RNA, single-stranded mRNA, or single-stranded non-coding RNA. In some embodiments, the single-stranded RNA is polyadenylated and/or the process comprises a polyadenylation step. In some embodiments, the RNA in the sample is capped and/or the process comprises capping the RNA in the sample. In some embodiments, the process comprises contacting the RNA sample with one or more oligonucleotide dT (oligo dT) probe(s) that bind polyadenylated RNA and removing unbound RNA from the sample.

In another aspect, an RNA purification process is provided, comprising: contacting an RNA sample comprising single-stranded polyadenylated RNA and double-stranded RNA (dsRNA) with a first oligonucleotide dT probe that binds polyadenylated RNA and removing unbound RNA from the sample; contacting the sample with an antibody or antigen binding fragment thereof that binds dsRNA; and purifying single-stranded polyadenylated RNA. In various embodiments, the single-stranded RNA is single-stranded circular RNA, single-stranded mRNA, or single-stranded non-coding RNA. In some embodiments, the single-stranded RNA is polyadenylated and/or the process comprises a polyadenylation step. In some embodiments, the RNA in the sample is capped and/or the process comprises capping the RNA in the sample.

In another aspect, an RNA purification process is provided, comprising: contacting an RNA sample comprising single-stranded polyadenylated RNA and double-stranded RNA (dsRNA) with a first oligonucleotide dT probe that binds polyadenylated RNA and removing unbound RNA from the sample; contacting the sample with an antibody or antigen binding fragment thereof that binds dsRNA, thus forming dsRNA:antibody complexes; and removing the dsRNA:antibody complexes from the sample; thereby purifying the single-stranded polyadenylated RNA. In various embodiments, the single-stranded RNA is single-stranded circular RNA, single-stranded mRNA, or single-stranded non-coding RNA. In some embodiments, the single-stranded RNA is polyadenylated and/or the process comprises a polyadenylation step. In some embodiments, the RNA in the sample is capped and/or the process comprises capping the RNA in the sample.

In another aspect, an RNA purification process is provided, comprising: contacting an RNA sample comprising single-stranded polyadenylated RNA and double-stranded RNA (dsRNA) with a first oligonucleotide dT probe that binds polyadenylated RNA and removing unbound RNA from the sample; contacting the sample with an antibody or antigen binding fragment thereof that binds dsRNA, thus forming dsRNA:antibody complexes; removing the dsRNA:antibody complexes from the sample; and contacting the sample with a second oligonucleotide dT probe to capture single-stranded polyadenylated RNA; thereby purifying the single-stranded polyadenylated RNA. In various embodiments, the single-stranded RNA is single-stranded circular RNA, single-stranded mRNA, or single-stranded non-coding RNA. In some embodiments, the single-stranded RNA is polyadenylated and/or the process comprises a polyadenylation step. In some embodiments, the RNA in the sample is capped and/or the process comprises capping the RNA in the sample.

In particular embodiments, the RNA is a therapeutic RNA (e.g., mRNA). In particular embodiments, the RNA encodes a therapeutic polypeptide.

In another aspect, a process for increasing nuclease editing efficiency is provided comprising: contacting an RNA sample comprising single-stranded polyadenylated RNA and double-stranded RNA (dsRNA) with a first oligonucleotide dT probe that binds polyadenylated RNA and removing unbound RNA from the sample; contacting the sample with an antibody or antigen binding fragment thereof that binds dsRNA; and purifying single-stranded polyadenylated RNA; wherein the editing rate of the nuclease is increased compared to the editing rate of a nuclease encoded by an RNA that is not contacted with an antibody that binds dsRNA. In various embodiments, the single-stranded RNA is single-stranded circular RNA, single-stranded mRNA, or single-stranded non-coding RNA. In some embodiments, the single-stranded RNA is polyadenylated and/or the process comprises a polyadenylation step. In some embodiments, the RNA in the sample is capped and/or the process comprises capping the RNA in the sample.

In another aspect, a process for increasing nuclease editing efficiency is provided comprising: contacting an RNA sample comprising single-stranded polyadenylated RNA and double-stranded RNA (dsRNA) with a first oligonucleotide dT probe that binds polyadenylated RNA and removing unbound RNA from the sample; contacting the sample with an antibody or antigen binding fragment thereof that binds dsRNA, thus forming dsRNA:antibody complexes; and removing the dsRNA:antibody complexes from the sample; wherein the editing rate of the nuclease is increased compared to the editing rate of a nuclease encoded by an RNA that is not contacted with an antibody that binds dsRNA. In various embodiments, the single-stranded RNA is single-stranded circular RNA, single-stranded mRNA, or single-stranded non-coding RNA. In some embodiments, the single-stranded RNA is polyadenylated and/or the process comprises a polyadenylation step. In some embodiments, the RNA in the sample is capped and/or the process comprises capping the RNA in the sample.

In another aspect, a process for increasing nuclease editing efficiency is provided comprising: contacting an RNA sample comprising single-stranded polyadenylated RNA and double-stranded RNA (dsRNA) with a first oligonucleotide dT probe that binds polyadenylated RNA and removing unbound RNA from the sample; contacting the sample with an antibody or antigen binding fragment thereof that binds dsRNA, thus forming dsRNA:antibody complexes; removing the dsRNA:antibody complexes from the sample; and contacting the sample with a second oligonucleotide dT probe to capture single-stranded polyadenylated RNA; wherein the editing rate of the nuclease is increased compared to the editing rate of a nuclease encoded by an RNA that is not contacted with an antibody that binds dsRNA and/or a second oligo dT. In various embodiments, the single-stranded RNA is single-stranded circular RNA, single-stranded mRNA, or single-stranded non-coding RNA. In some embodiments, the single-stranded RNA is polyadenylated and/or the process comprises a polyadenylation step. In some embodiments, the RNA in the sample is capped and/or the process comprises capping the RNA in the sample.

In various embodiments, nuclease is an endonuclease or exonuclease. In some embodiments, the endonuclease is a homing endonuclease, megaTAL, CRISPR-associated nuclease (e.g., Cas9 and variants thereof), zinc finger nuclease, transcription activator-like effector nuclease (TALEN).

In another aspect, a process for decreasing the immunogenicity and/or toxicity of RNA administered to a cell or subject is provided comprising: contacting an RNA sample comprising single-stranded polyadenylated RNA and double-stranded RNA (dsRNA) with a first oligonucleotide dT probe that binds polyadenylated RNA and removing unbound RNA from the sample; contacting the sample with an antibody or antigen binding fragment thereof that binds dsRNA; and purifying single-stranded polyadenylated RNA; wherein the immunogenicity and/or toxicity of the RNA, when administered to a cell or subject, is less than the immunogenicity and/or toxicity of an RNA administered to a cell or subject, when the RNA has not been contacted with an antibody that binds dsRNA. In various embodiments, the single-stranded RNA is single-stranded circular RNA, single-stranded mRNA, or single-stranded non-coding RNA. In some embodiments, the single-stranded RNA is polyadenylated and/or the process comprises a polyadenylation step. In some embodiments, the RNA in the sample is capped and/or the process comprises capping the RNA in the sample.

In another aspect, a process for decreasing the immunogenicity and/or toxicity of RNA administered to a cell or subject is provided comprising: contacting an RNA sample comprising single-stranded polyadenylated RNA and double-stranded RNA (dsRNA) with a first oligonucleotide dT probe that binds polyadenylated RNA and removing unbound RNA from the sample; contacting the sample with an antibody or antigen binding fragment thereof that binds dsRNA, thus forming dsRNA:antibody complexes; and removing the dsRNA:antibody complexes from the sample; wherein the immunogenicity and/or toxicity of the RNA, when administered to a cell or subject, is less than the immunogenicity and/or toxicity of an mRNA administered to a cell or subject, when the RNA has not been contacted with an antibody that binds dsRNA. In various embodiments, the single-stranded RNA is single-stranded circular RNA, single-stranded mRNA, or single-stranded non-coding RNA. In some embodiments, the single-stranded RNA is polyadenylated and/or the process comprises a polyadenylation step. In some embodiments, the RNA in the sample is capped and/or the process comprises capping the RNA in the sample.

In another aspect, a process for decreasing the immunogenicity and/or toxicity of RNA administered to a cell or subject is provided comprising: contacting an RNA sample comprising single-stranded polyadenylated RNA and double-stranded RNA (dsRNA) with a first oligonucleotide dT probe that binds polyadenylated RNA and removing unbound RNA from the sample; contacting the sample with an antibody or antigen binding fragment thereof that binds dsRNA, thus forming dsRNA:antibody complexes; removing the dsRNA:antibody complexes from the sample; and contacting the sample with a second oligonucleotide dT probe to capture single-stranded polyadenylated RNA; wherein the immunogenicity and/or toxicity of the RNA, when administered to a cell or subject, is less than the immunogenicity and/or toxicity of an RNA administered to a cell or subject, when the RNA has not been contacted with an antibody that binds dsRNA. In various embodiments, the single-stranded RNA is single-stranded circular RNA, single-stranded mRNA, or single-stranded non-coding RNA. In some embodiments, the single-stranded RNA is polyadenylated and/or the process comprises a polyadenylation step. In some embodiments, the RNA in the sample is capped and/or the process comprises capping the RNA in the sample.

In any of the embodiments contemplated herein, the anti-dsRNA antibody is selected from the group consisting of: J2, J5, K1, K2, 1D3, CABT-B212, and 9D5, or a functional derivative or fragment thereof. In particular embodiments, the anti-dsRNA antibody is J2.

In any of the embodiments, methods, procedures, or processes contemplated herein may comprise additional steps, e.g., plasmid linearization/digestion, in vitro transcription, diafiltration, ultra-filtration, and final filtration.

Techniques for recombinant (i.e., engineered) DNA, peptide and oligonucleotide synthesis, immunoassay's, tissue culture, transformation (e.g., electroporation, lipofection), enzymatic reactions, purification and related techniques and procedures may be generally performed as described in various general and more specific references in microbiology, molecular biology, biochemistry, molecular genetics, cell biology, virology and immunology as cited and discussed throughout the present specification. See, e.g., Sambrook et al., Molecular Cloning: A Laboratory Manual, 3d ed., Cold Spring Harbor Laboratory Press, Cold Spring Harbor, N.Y.; Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford Univ. Press USA, 1985); Current Protocols in Immunology (Edited by: John E. Coligan, Ada M. Kruisbeek, David H. Margulies, Ethan M. Shevach, Warren Strober 2001 John Wiley & Sons, NY, NY); Real-Time PCR: Current Technology and Applications, Edited by Julie Logan, Kirstin Edwards and Nick Saunders, 2009, Caister Academic Press, Norfolk, UK; Anand, Techniques for the Analysis of Complex Genomes, (Academic Press, New York, 1992); Guthrie and Fink, Guide to Yeast Genetics and Molecular Biology (Academic Press, New York, 1991); Oligonucleotide Synthesis (N. Gait, Ed., 1984); Nucleic Acid The Hybridization (B. Hames & S. Higgins, Eds., 1985); Transcription and Translation (B. Hames & S. Higgins, Eds., 1984); Animal Cell Culture (R. Freshney, Ed., 1986); Perbal, A Practical Guide to Molecular Cloning (1984); Next-Generation Genome Sequencing (Janitz, 2008 Wiley-VCH); PCR Protocols (Methods in Molecular Biology) (Park, Ed., 3rd Edition, 2010 Humana Press); Immobilized Cells And Enzymes (IRL Press, 1986); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Harlow and Lane, Antibodies, (Cold Spring Harbor Laboratory Press, Cold Spring Harbor, N.Y., 1998); Immunochemical Methods In Cell And Molecular Biology (Mayer and Walker, eds., Academic Press, London, 1987); Handbook Of Experimental Immunology, Volumes I-IV (D. M. Weir and C C Blackwell, eds., 1986); Roitt, Essential Immunology, 6th Edition, (Blackwell Scientific Publications, Oxford, 1988); Current Protocols in Immunology (Q. E. Coligan, A. M. Kruisbeek, D. H. Margulies, E. M. Shevach and W. Strober, eds., 1991); Annual Review of Immunology; as well as monographs in journals such as Advances in Immunology.

### B. Definitions

Prior to setting forth this disclosure in more detail, it may be helpful to an understanding thereof to provide definitions of certain terms to be used herein.

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below.

The articles “a,” “an,” and “the” are used herein to refer to one or to more than one (i.e., to at least one, or to one or more) of the grammatical object of the article. By way of example, “an element” means one element or one or more elements.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

In one embodiment, a range, e.g., 1 to 5, about 1 to 5, or about 1 to about 5, refers to each numerical value encompassed by the range. For example, in one non-limiting and merely illustrative embodiment, the range “1 to 5” is equivalent to the expression 1, 2, 3, 4, 5; or 1.0, 1.5, 2.0, 2.5, 3.0, 3.5, 4.0, 4.5, or 5.0; or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher compared to a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are present that materially affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

The term “ex vivo” refers generally to activities that take place outside an organism, such as experimentation or measurements done in or on living tissue in an artificial environment outside the organism, preferably with minimum alteration of the natural conditions. In particular embodiments, “ex vivo” procedures involve living cells or tissues taken from an organism and cultured or modulated in a laboratory apparatus, usually under sterile conditions, and typically for a few hours or up to about 24 hours, but including up to 48 or 72 hours, depending on the circumstances. In certain embodiments, such tissues or cells can be collected and frozen, and later thawed for ex vivo treatment. Tissue culture experiments or procedures lasting longer than a few days using living cells or tissue are typically considered to be “in vitro,” though in certain embodiments, this term can be used interchangeably with ex vivo.

The term “in vivo” refers generally to activities that take place inside an organism. In one embodiment, cellular genomes are engineered, edited, or modified in vivo.

An “increased” or “enhanced” amount is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or control.

A “decrease” or “reduced” amount is typically a “statistically significant” amount, and may include a decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, or control.

By “maintain,” or “preserve,” or “maintenance,” or “no change,” or “no substantial change,” or “no substantial decrease” refers to a response that is not significantly different or measurably different from a reference response, vehicle or control.

The terms “specific binding affinity” or “specifically binds” or “specifically bound” or “specific binding” or “specifically targets” as used herein, describe binding of one molecule to another, e.g., an antibody binding to double stranded RNA (dsRNA), a nucleotide (e.g., oligo dT) binding to a poly(A)-tail, or a meganuclease (or binding domain) binding to a target site, at greater binding affinity than background binding. An antibody, nucleotide, or binding domain “specifically binds” to another molecule (e.g., DNA, RNA, or polypeptide) if it binds to or associates with the molecule with an affinity or Kₐ (i.e., an equilibrium association constant of a particular binding interaction with units of 1/M) of, for example, greater than or equal to about 10⁵ M⁻¹. In certain embodiments, a binding domain binds to a target site with a Kₐ greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding domains refers to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater.

The terms “selectively binds” or “selectively bound” or “selectively binding” or “selectively targets” and describe preferential binding of one molecule to a target molecule (on-target binding) in the presence of a plurality of off-target molecules. In particular embodiments, an anti-dsRNA antibody, or fragment thereof, selectively binds to dsRNA about 5, 10, 15, 20, 25, 50, 100, or 1000 times more frequently than the anti-dsRNA antibody binds single-stranded RNA (ssRNA).

The term “antibody” refers to a binding agent that is a polypeptide comprising at least a light chain or heavy chain immunoglobulin variable region or fragment thereof which specifically recognizes and binds one or more epitopes of an antigen, such as a peptide, lipid, polysaccharide, or nucleic acid containing an antigenic determinant, such as those recognized by an immune cell. In some embodiments, the antibody is an anti-dsRNA antibody. In particular embodiments, the anti-dsRNA antibody and dsRNA form a dsRNA:antibody complex.

The term “antibody” encompasses any naturally-occurring, recombinant, modified or engineered immunoglobulin or immunoglobulin-like structure or antigen-binding fragment or portion thereof, or derivative thereof, as further described elsewhere herein. Thus, the term refers to an immunoglobulin molecule that specifically binds to a target antigen, and includes, for instance, chimeric, humanized, fully human, and bispecific antibodies. An intact antibody will generally comprise at least two full-length heavy chains and two full-length light chains, but in some instances can include fewer chains such as antibodies naturally occurring in camelids which can comprise only heavy chains. Antibodies can be derived solely from a single source, or can be “chimeric,” that is, different portions of the antibody can be derived from two different antibodies. Antibodies, or antigen-binding portions thereof, can be produced in hybridomas, by recombinant DNA techniques, or by enzymatic or chemical cleavage of intact antibodies.

The term “antigen-binding fragment” or “antigen-binding portion” refers to one or more fragments of an antibody that retain the ability to specifically bind to an antigen (e.g., dsRNA). Antigen binding fragments include, but are not limited to, any naturally occurring, enzymatically obtainable, synthetic, or genetically engineered polypeptide or glycoprotein that specifically binds an antigen to form a complex. In some embodiments, an antigen-binding portion of an antibody may be derived, e.g., from full antibody molecules using any suitable standard techniques such as proteolytic digestion or recombinant genetic engineering techniques involving the manipulation and expression of DNA encoding antibody variable and optionally constant domains.

An “isolated antibody or antigen binding fragment thereof” refers to an antibody or antigen binding fragment thereof which has been identified and separated and/or recovered from a component of its natural environment.

As used herein, “gene of interest” or “polynucleotide of interest” refers to a polynucleotide which encodes a polypeptide or protein of interest. Depending on the context, the gene of interest refers to a deoxyribonucleic acid, e.g., a gene of interest in a DNA template which can be transcribed to an RNA transcript, or a ribonucleic acid, e.g., a gene of interest in an RNA transcript which can be translated to produce the encoded polypeptide of interest in vitro, in vivo, in situ or ex vivo. As described in more detail below, a polypeptide of interest includes but is not limited to, biologics, antibodies, vaccines, therapeutic proteins or peptides, endonucleases, exonucleases, etc.

As used herein, the term “operably linked”, refers to a juxtaposition wherein the components described are in a relationship permitting them to function in their intended manner. In one embodiment, the term refers to a functional linkage between a nucleic acid expression control sequence (such as a promoter, and/or enhancer) and a second polynucleotide sequence, e.g., a polynucleotide coding for a gene of interest, wherein the expression control sequence directs transcription of the nucleic acid corresponding to the second sequence. For example, a gene of interest operably linked to an RNA polymerase promoter allows transcription of the gene of interest.

As used herein, the terms “polypeptide,” “polypeptide fragment,” “peptide” and “protein” are used interchangeably, unless specified to the contrary, and according to conventional meaning, i.e., as a sequence of amino acids. Polypeptides include “polypeptide variants.” Polypeptide variants may differ from a naturally occurring polypeptide in one or more amino acid substitutions, deletions, additions and/or insertions. Such variants may be naturally occurring or may be synthetically generated, for example, by modifying one or more amino acids of the polypeptide sequence(s).

As used herein, “poly A tail”, “poly(A) tail”, or “poly(A)” refers to a chain of adenine nucleotides. The term can refer to poly(A) tail that is to be added to an RNA transcript, or can refer to the poly(A) tail that already exists at the 3′ end of an RNA transcript (e.g., a DNA encoded poly(A) tail). As described in more detail below, a poly(A) tail is typically 5-300 nucleotides in length (SEQ ID NO: 13).

The term “polynucleotide” is interchangeable with the term “nucleic acid”, and includes any compound and/or substance that comprise a polymer of nucleotides. Thus, the terms “polynucleotide” or “nucleic acid” include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

Additional definitions are set forth throughout this disclosure.

### C. Methods

As discussed throughout this disclosure, the inventors recognized that dsRNA is responsible for increased immunogenicity and increased cellular toxicity in RNA compositions, particularly in the context of in vivo therapeutic RNA applications. Moreover, the inventors have surprisingly discovered an improved method/process to remove dsRNA from RNA preparations.

Whether the method is for RNA purification generally or for particular applications (e.g., in vivo therapeutic mRNA treatment or a process for improving gene editing in vivo), the method generally comprises: contacting an RNA sample comprising single-stranded RNA and double-stranded RNA (dsRNA) with an antibody or antigen binding fragment thereof that binds dsRNA; and purifying single-stranded RNA.

In one aspect, the process comprises: contacting an RNA sample comprising single-stranded polyadenylated RNA and double-stranded RNA (dsRNA) with a first oligonucleotide dT probe that binds polyadenylated RNA and removing unbound RNA from the sample; contacting the sample with an antibody or antigen binding fragment thereof that binds dsRNA; and purifying single-stranded polyadenylated RNA.

In another aspect, the process comprises: contacting an RNA sample comprising single-stranded polyadenylated RNA and double-stranded RNA (dsRNA) with a first oligonucleotide dT probe that binds polyadenylated RNA and removing unbound RNA from the sample; contacting the sample with an antibody or antigen binding fragment thereof that binds dsRNA, thus forming dsRNA:antibody complexes; and removing the dsRNA:antibody complexes from the sample; thereby purifying the single-stranded polyadenylated RNA.

In yet another aspect, the process comprises: contacting an RNA sample comprising single-stranded polyadenylated RNA and double-stranded RNA (dsRNA) with a first oligonucleotide dT probe that binds polyadenylated RNA and removing unbound RNA from the sample; contacting the sample with an antibody or antigen binding fragment thereof that binds dsRNA, thus forming dsRNA:antibody complexes; removing the dsRNA:antibody complexes from the sample; and contacting the sample with a second oligonucleotide dT probe to capture single-stranded polyadenylated mRNA; thereby purifying the single-stranded, capped, and polyadenylated RNA.

As described further below, the method may comprise other steps, e.g., plasmid linearization/digestion, in vitro transcription, polyadenylation, capping, diafiltration, ultra-filtration, and final filtration. In some embodiments, the RNA can be for use in in vitro, ex vivo, or in vivo methods. In some embodiments, the single-stranded RNA is single-stranded circular RNA, single-stranded mRNA, or single-stranded non-coding RNA. In particular embodiments, the RNA is an mRNA.

### 1. DNA

The methods disclosed herein must have a source of RNA. The RNA can be obtained or isolated from cells or tissues. Alternatively, the RNA may be prepared de novo through chemical synthesis. In various embodiments, the RNA may be transcribed in vitro from a source of DNA (e.g., isolated genomic DNA, plasmid DNA, or linear/linearized DNA). In various embodiments, the RNA is obtained from an in vitro transcription (IVT) assay using linearized plasmid DNA. In some embodiments, the RNA is obtained from an in vitro transcription (IVT) assay using a linear DNA/vector.

As used herein, the term “plasmid DNA” or “plasmid DNA vector” refers to a circular nucleic acid molecule, preferably to an artificial/recombinant DNA molecule. In some embodiments, a plasmid DNA vector can be linearized. Alternatively, a “linear DNA”, “linear DNA vector”, or “linear vector”, refers to a linear nucleic acid molecule, preferably to an artificial/recombinant linear DNA molecule. A plasmid or linear DNA vector in the context of the present disclosure is suitable for incorporating or harboring a desired nucleic acid sequence or gene of interest, such as a nucleic acid sequence comprising a sequence encoding an RNA, and/or an open reading frame (ORF), encoding at least one polypeptide or gene of interest. Exemplary plasmids useful in the methods described herein include, but are not limited to pUC-based vectors, e.g., pUC19. Exemplary linear DNA vectors linear include but are not limited to pJAZZ®, pSMART® (Lucigen™), and Doggybone™/dbDNA™ (Touchlight) vectors.

An expression vector may be used for production of expression products such as RNA, e.g., mRNA in a process called RNA in vitro transcription. For example, an expression vector may comprise sequences needed for RNA in vitro transcription of a sequence stretch of the vector, such as a promoter sequence, e.g., an RNA promoter sequence.

Preferably, a DNA vector comprises a multiple cloning site, an RNA promoter sequence, an RNA poly(A) tail, optionally a selection marker (such as an antibiotic resistance factor), and a sequence suitable for multiplication of the vector, such as an origin of replication. In particular embodiments, the DNA vector, or expression vector, comprises a promoter for DNA-dependent RNA polymerase, e.g., T3, T7 and Sp6. Plasmid DNA may also comprise a restriction site for linearization.

As used herein, the term “template DNA” (or “DNA template”) refers to a DNA molecule comprising a nucleic acid sequence encoding the RNA sequence to be in vitro transcribed. Therefore, the template DNA comprises all elements necessary for in vitro transcription, particularly a promoter element for binding of a DNA dependent RNA polymerase, e.g., T3, T7 and SP6 RNA polymerases 5′ of the DNA sequence encoding the target RNA sequence, and operably linked there to. The template DNA may also include a sequence coding for a poly(A) tail located 3′ to the gene of interest.

Methods for generating, replicating, and cloning of the recombinant template or plasmid DNA describe herein are known in the art.

The term “template DNA” may also refer to a plasmid DNA vector which comprises a nucleic acid sequence encoding the RNA sequence. Furthermore, the “template DNA” may be a linear or a circular DNA molecule. In particular embodiments, the template DNA is a linearized/digested plasmid DNA molecule.

A linearized template DNA plasmid can be obtained by contacting the plasmid DNA with a restriction enzyme under suitable conditions so that the restriction enzyme cuts the plasmid DNA at its recognition site(s) and disrupts the plasmid structure. If the plasmid DNA contains only one recognition site for the restriction enzyme, the linearized template DNA has the same number of nucleotides as the plasmid DNA. If the plasmid DNA contains more than one recognition site for the restriction enzyme, the linearized template DNA has a smaller number of nucleotides than the plasmid DNA. The linearized template DNA is then the fragment of the plasmid DNA, which contains the elements necessary for RNA in vitro transcription, that is a promoter element for RNA transcription and the template DNA element. Restriction enzymes suitable for cutting DNA and/or linearization of plasmid DNA are known in the art, including, but not limited to, BciVI, XbaI, SpeI, HindIII, NotI, EcoRI, NdeI, BsaI, AfIII, HindIII, and SapI. In some embodiments, the restriction enzyme is a type IIS restriction enzyme. Type IIS restriction enzymes include, but are not limited to AcuI, ALI, BoaeI, BbsI, BbsI-HF, BbvI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspMI, MspQI, BsrDI, BsrI, BtgZI, BtsCI, BtsI, CspCI, EarI, EciI, Esp3I, FauI, FokI, HgaI, HphhI, Hpy AV, MboII, MlyI, MmeI, MnlI, NmeAIII, PaqCI, PleI, PpiI, PsrI, SapI, SfaNI. In particular embodiments, the restriction enzyme is BsaI.

Linear DNA vectors/templates may also be subject to restriction by endonucleases. In some embodiments, a linear DNA vector/template is contacted with a restriction enzyme to produce terminal adenine (A) nucleotides.

In some embodiments, following restriction, the plasmid or linear DNA template is filtered (e.g., by ultrafiltration and/or diafiltration) into an appropriate solvent, e.g., water, TE (Tris-EDTA), Tris HCl pH 7.5, HEPES/phosphate and the like.

The linearized or linear DNA template can be purified before use as a template for in vitro transcription. For example, the linearized or linear DNA template can be purified by phenol/chloroform extraction with subsequent alcohol precipitation, chromatographic methods or filtration methods, or silica-based DNA capture methods.

This step also ensures the reduction of impurities (e.g., proteins) from the previous manufacturing steps, including E. coli proteins, restriction enzymes and BSA (contained in reaction buffers).

In various embodiments, the process further comprises a step of treating the sample with a DNase to remove residual plasmid DNA template (circular or linear residual DNA). In some embodiments, the DNase treatment step occurs after an IVT step and/or after a capping step. In particular embodiments, the DNase is DNase I.

### 2. RNA Production

In particular embodiments, linearized DNA may be used in an in vitro transcription (IVT) system to generate RNA for use in the methods describe herein. The IVT system typically comprises a transcription buffer, nucleotide triphosphates (NTPs), an RNase inhibitor and an RNA polymerase. Methods for in vitro transcription are known in the art. See, e.g., Beckert et al., Methods Mol Biol. 2011; 703:29-41. Exemplary commercially supplied kits for IVT include, but are not limited to, HiScribe™ T7 Quick High Yield RNA Synthesis Kit (New England BioLabs™), MEGAscript® T7 Kit (ThermoFisher Scientific™), TranscriptAid T7 High Yield Transcription Kit (ThermoFisher Scientific™), Riboprobe® or RiboMAX™ RNA Production System (Promega™), AmpliScribe™ T7 Transcription kits (Lucigen®), and RNAMaxx™ (Agilent Technologies™).

Alternatively, IVT assays can be assembled and performed in-house by obtaining each component separately and using methods known in the art. The NTPs may be manufactured in house or purchase from commercial suppliers (e.g., Trilink® and NewEngland BioLabs®). Any number of RNA polymerases or variants thereof may be used in the method described herein, and are readily available through commercial suppliers (e.g., NewEngland BioLabs®, ThermoFisher Scientific™, and MilliporeSigma™). The polymerase may be selected from, but is not limited to, a phage RNA polymerase, e.g., a T7 RNA polymerase, a T3 RNA polymerase, an SP6 RNA polymerase, and/or mutant polymerases such as, but not limited to, polymerases able to incorporate modified nucleic acids.

A typical in vitro transcription reaction includes the following: an RNA polymerase, e.g., a T7 RNA polymerase; a DNA template; nucleotides (NTPs); MgCl2; and a buffer such as, e.g., HEPES or Tris. IVT reactions can also include dithiothreitol (DTT) and/or spermidine, an RNase inhibitor, a pyrophosphatase, and/or EDTA. The in vitro transcription reaction is allowed to proceed, for example, under constant mixing at 37° C. for 4 hours.

### 3. Capping Reaction

In some embodiments, the RNA used in the methods described herein is capped. Capping RNA maximizes efficiency of expression in cells by increasing stability and reducing degradation. In some embodiments, the RNA molecules used in the methods are synthesized in vitro by incubating uncapped RNA in the presence a capping enzyme system. In some embodiments, the RNA is enzymatically capped at the 5′ end after in vitro transcription. In some embodiments, the RNA is enzymatically capped at the 5′ end co-transcriptionally. Accordingly, capping can be performed either before or after further purification of the RNA, e.g., oligo dT purification. In some embodiments, oligo dT affinity purification and ultrafiltration/diafiltration is performed prior to the capping reaction.

As used herein, the terms “5′ cap” or “5′ cap structure” or “5′ cap moiety” refer to a chemical modification, which has been incorporated at the 5′ end of an mRNA. The 5′ cap is involved in nuclear export, mRNA stability, and translation.

In particular embodiments, an mRNA contemplated herein comprises a 5′ cap comprising a 5′-ppp-5′-triphosphate linkage between a terminal guanosine cap residue and the 5′-terminal transcribed sense nucleotide of the mRNA molecule. This 5′-guanylate cap may then be methylated to generate an N7-methyl-guanylate residue.

Illustrative examples of 5′ cap suitable for use in particular embodiments of the mRNA polynucleotides contemplated herein include, but are not limited to: unmethylated 5′ cap analogs, e.g. G(5′)ppp(5′)G, G(5′)ppp(5′)C, G(5′)ppp(5′)A; methylated 5′ cap analogs, e.g. m⁷G(5′)ppp(5′)G, m⁷G(5′)ppp(5′)C, and m⁷G(5′)ppp(5′)A; dimethylated 5′ cap analogs, e.g., m^{2,7}G(5′)ppp(5′)G, n^{2,7}G(5′)ppp(5′)C, and m^{2,7}G(5′)ppp(5′)A; trimethylated 5′ cap analogs, e.g., m^{2,2,7}G(5′)ppp(5′)G, m^{2,2,7}G(5′)ppp(5′)C, and m^{2,2,1}G(5′)ppp(5′)A; dimethylated symmetrical 5′ cap analogs, e.g., m⁷G(5′)pppm⁷(5′)G, m⁷G(5′)pppm⁷(5′)C, and m⁷G(5′)pppm⁷(5′)A; and anti-reverse 5′ cap analogs, e.g. Anti-Reverse Cap Analog (ARCA) cap, designated 3′O-Me-m⁷G(5′)ppp(5′)G, 2′O-Me-m⁷G(5′)ppp(5′)G, 2′O-Me-m⁷G(5′)ppp(5′)C, 2′O-Me-m⁷G(5′)ppp(5′)A, m⁷2′d(5′)ppp(5′)G, m⁷2′d(5′)ppp(5′)C, m⁷2′d(5′)ppp(5′)A, 3′O-Me-m⁷G(5′)ppp(5′)C, 3′O-Me-m⁷G(5′)ppp(5′)A, m⁷3′d(5′)ppp(5′)G, m⁷3′d(5′)ppp(5′)C, m⁷3′d(5′)ppp(5′)A and their tetraphosphate derivatives) (see, e.g., Jemielity et al., RNA, 9: 1108-1122 (2003)).

In particular embodiments, mRNAs comprise a 5′ cap that is a 7-methyl guanylate (“m⁷G”) linked via a triphosphate bridge to the 5′-end of the first transcribed nucleotide, resulting in m⁷G(5′)ppp(5′)N, where N is any nucleoside.

In some embodiments, mRNAs comprise a 5′ cap wherein the cap is a Cap0 structure (Cap0 structures lack a 2′-O-methyl residue of the ribose attached to bases 1 and 2), a Cap1 structure (Cap1 structures have a 2′-O-methyl residue at base 2), or a Cap2 structure (Cap2 structures have a 2′-O-methyl residue attached to both bases 2 and 3).

For example, the RNA can be enzymatically capped at the 5′ end using Vaccinia guanylyltransferase, guanosine triphosphate and s-adenosyl-L-methionine to yield cap 0 structure. An inverted 7-methylguanosine cap is added via a 5′ to 5′ triphosphate bridge. Alternatively, use of a 2′O-methyltransferase with Vaccinia guanylyltransferase yields the cap 1 structure where in addition to the cap 0 structure, the 2′OH group is methylated on the penultimate nucleotide. S-adenosyl-L-methionine (SAM) is a cofactor utilized as a methyl transfer reagent.

In one embodiment, an mRNA comprises a m⁷G(5′)ppp(5′)G cap.

In one embodiment, an mRNA comprises an ARCA cap or modified ARCA cap.

In various embodiments, the RNA is co-transcriptionally capped or enzymatically capped in a separate reaction. The 5′ terminal caps may include endogenous caps or cap analogs. A 5′ terminal cap may comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

Further examples of 5′ cap structures include glyceryl, inverted deoxy abasic residue (moiety), 4′,5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxy butyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′ phosphorothioate, phosphorodithioate, or bridging or non-bridging methylphosphonate moiety. Further modified 5′-CAP structures which may be used in the context of the present invention are CAP1 (methylation of the ribose of the adjacent nucleotide of m7GpppN), CAP2 (methylation of the ribose of the 2nd nucleotide downstream of the m7GpppN), CAP3 (methylation of the ribose of the 3rd nucleotide downstream of the m7GpppN), CAP4 (methylation of the ribose of the 4th nucleotide downstream of the m7GpppN), ARCA (anti-reverse CAP analogue, modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

In eukaryotes, at least three enzymatic activities are required to generate a functional cap 0) (RNA triphosphatase (TPase), RNA guanylyltransferase (GTase) and guanine-N7 methyltransferase (guanine-N7 MTase). For a cap 1 structure, an additional m⁷G-specific 2′O methyltransferase (2′O MTase) is required to methylate the +1 ribonucleotide at the 2′O position of the ribose. Eukaryote capping enzymes are known in the art (Nucleic Acids Research, Volume 44, Issue 16, 19 Sep. 2016, Pages 7511-7526).

Viral RNA capping enzymes are also known in the art. In some instances, viral capping enzymes are known to couple enzymatic activities into multifunctional proteins. For example, Flavivirus, Dengue, West Nile, and Paramyxoviruses, couple the GTase and MTase activities into their RNA polymerase (RdRp). Alternatively, the Vaccinia virus capping enzyme and Bluetongue virus capping enzyme couple all the necessary enzymatic activities of RNA capping to generate cap 0 or cap 1. Thus, due to its simplicity and effectiveness, the Vaccinia virus capping enzyme Vaccinia guanylyltransferase is often a preferred capping enzyme, but not a requirement. Other viral capping enzymes known in the art include, but are not limited to, chlorella virus, alpha virus, rhabdovirus, and vesicular stomatitis virus capping enzymes. In some embodiments, the polyadenylated mRNA is capped at its 5′ end using a Vaccinia guanylyltransferase, guanosine triphosphate, and S-adenosyl-L-methionine (SAM) to produce a cap 0 structure. In some embodiments, the polyadenylated mRNA is capped at its 5′ end using a Vaccinia guanylyltransferase, guanosine triphosphate, S-adenosyl-L-methionine (SAM), and an 2′-O-Methyltransferase to produce a cap 1 structure.

Capping methods and conditions are known in the art (see, e.g., Wiley Interdiscip Rev RNA, 2010 July-August; 1(1); 152-172; and Nat Rev Microbiol. 2011 Dec. 5; 10(1):51-65. An exemplary capping reaction may include the following: S-adenosylmethionine chloride (SAM); RNase inhibitor; buffer (e.g., NEB capping buffer); GTP; Vaccinia Enzyme; mRNA Cap 2′-O-Methyltransferase; and EDTA. The reaction is run under constant mixing at 37° C.

### 4. Affinity Chromatography

To date, the primary means for purifying biological products such as monoclonal antibodies, therapeutic proteins, vaccines, and other biological products (including DNA and RNA) has been through the use of capture chromatography, e.g., affinity chromatography. As used herein, the terms “capture chromatography” and “affinity chromatography” refer to a chromatography component, or related method step, which involves binding and eluting of a desired product (e.g., RNA) to and from a column. Capture or affinity chromatography typically uses selective non-covalent interactions between an analyte and specific molecule(s) (e.g., a specific ligand coupled to a chromatographic medium). For example, capture or affinity chromatography may use protein A, protein G, an antibody (e.g., anti-dsRNA antibody), a specific substrate/probe (e.g., oligo dT), ligand or antigen as the capture reagent. The capture reagent is then mobilized (linked or bound) to a resin/surface within a column, and the sample is passed over the resin/surface (i.e., through the column). The bound product is then eluted off of the column.

Systems for chromatography (e.g., liquid chromatography) are known by those skilled in the art, e.g., high performance liquid chromatography (HPLC), ultra-high performance liquid chromatography (UHPLC), and fast protein liquid chromatography (FPLC; e.g., AKTA™ systems). Chromatography columns suitable for use in the methods described herein are also known by those skilled in the art. The column(s) may be of any suitable volume/size, e.g., 0.2 mL, 1.0 mL, 5.0 mL, or 10 mL. Exemplary manufacturers of chromatographic columns, systems, and materials include, but are not limited to, Sigma-Aldrich™, ThermoFisher Scientific™, Waters™, Bio-Rad Laboratories, PerkinElmer®, and Cytiva.

The column(s) may comprise a suitable resin/surface to retain the substrate or probe. Suitable resin/surface materials are known in the art. Exemplary materials that can be used as a surface include, but are not limited to acrylics, carbon (e.g., graphite, carbon-fiber), cellulose (e.g., cellulose acetate), ceramics, controlled-pore glass, cross-linked polysaccharides (e.g., agarose or SEPHAROSE™), gels, glass (e.g., modified or functionalized glass), gold (e.g., atomically smooth Au(111)), graphite, inorganic glasses, inorganic polymers, latex, metal oxides (e.g., Si02, Ti02, stainless steel), metalloids, metals (e.g., atomically smooth Au(111)), mica, molybdenum sulfides, nanomaterials (e.g., highly oriented pyrolitic graphite (HOPG) nanosheets), nitrocellulose, NYLON™, optical fiber bundles, organic polymers, paper, plastics, polacryloylmorpholide, poly(4-methylbutene), polyethylene terephthalate), poly(vinyl butyrate), polybutylene, polydimethylsiloxane (PDMS), polyethylene, polyformaldehyde, polymethacrylate, polypropylene, polysaccharides, polystyrene, poly(styrene-divinylbenzene), polyurethanes, polyvinylidene difluoride (PVDF), quartz, rayon, resins, beads, rubbers, semiconductor material, silica, silicon (e.g., surface-oxidized silicon), sulfide, and TEFLON™.

In various embodiments the RNA is purified via chromatographic methods using an oligo deoxythymidine (dT) probe or substrate. The mechanism of purification involves hybridization of the poly(A) tail of the RNA to the oligonucleotide ligand (oligo dT) under high salt conditions. The DNA template and/or other impurities will not bind. In addition, RNA transcripts that do not contain poly(A) stretches will not bind to the resin and will not form a duplex with the affinity ligand. Polyadenylated RNA can then be eluted from the resin utilizing a low ionic strength buffer or a competitive binding oligonucleotide solution. Accordingly, in some embodiments, the method comprises contacting an RNA sample with a first or second oligo dT probe/substrate mobilized within a chromatographic column, thus forming oligo dT:polyadenylated RNA complexes. In some embodiments, the method comprises separating unbound RNA and/or contaminants from the oligo dT:polyadenylated RNA complexes. In some embodiments, the method comprises eluting the polyadenylated RNA off the column and retaining the eluted RNA for further purification.

In particular embodiments, the method comprises: contacting an RNA sample comprising single-stranded polyadenylated RNA and double-stranded RNA (dsRNA) with a first oligonucleotide dT probe that binds polyadenylated RNA and removing unbound RNA from the sample; contacting the sample with an antibody or antigen binding fragment thereof that binds dsRNA; and purifying single-stranded polyadenylated mRNA.

In some embodiments, the method comprises: contacting an RNA sample comprising single-stranded polyadenylated RNA and double-stranded RNA (dsRNA) with a first oligonucleotide dT probe that binds polyadenylated RNA and removing unbound RNA from the sample; contacting the sample with an antibody or antigen binding fragment thereof that binds dsRNA, thus forming dsRNA:antibody complexes; and removing the dsRNA:antibody complexes from the sample; thereby purifying the single-stranded polyadenylated RNA.

In various embodiments, the methods described herein comprise more than one oligo dT probe or purification step. In some embodiments, the methods described herein comprise 2 oligo dT probes or purification steps. In some embodiments, the method comprises contacting a sample comprising RNA and double stranded RNA (dsRNA) with a first oligonucleotide dT probe that binds polyadenylated RNA. In some embodiments, the polyadenylated RNA separated from the dsRNA:antibody complex is contacted with a second oligonucleotide dT probe using affinity chromatography.

In particular embodiments, the method comprises: contacting an RNA sample comprising single-stranded polyadenylated RNA and double-stranded RNA (dsRNA) with a first oligonucleotide dT probe that binds polyadenylated RNA and removing unbound RNA from the sample; contacting the sample with an antibody or antigen binding fragment thereof that binds dsRNA, thus forming dsRNA:antibody complexes; removing the dsRNA:antibody complexes from the sample; and contacting the sample with a second oligonucleotide dT probe to capture single-stranded polyadenylated RNA; thereby purifying the single-stranded polyadenylated mRNA.

In some embodiments, the first and/or second oligonucleotide dT probe is bound to a surface. In some embodiments, the first oligonucleotide dT probe is bound to a surface. In some embodiments, the second oligonucleotide dT probe is bound to a surface. In some embodiments, the oligo dT probe is bound or covalently linked to a cellulose resin. In some embodiments, a prepacked oligo dT column is used. Prepacked oligo dT columns for chromatography are known in the art and are commercially available, e.g., POROS™ GoPure™ (ThermoFisher Scientific).

Oligo dT substrates/probes may be of different lengths, e.g., may comprise about 15 to about 30 thymidine residues. In some embodiments, the oligo dT substrate/probe comprises about 16 to about 30 thymidine residues. In some embodiments, the oligo dT substrate/probe comprises about 17 to about 30 thymidine residues. In some embodiments, the oligo dT substrate/probe comprises about 18 to about 30 thymidine residues. In some embodiments, the oligo dT substrate/probe comprises about 19 to about 30 thymidine residues. In some embodiments, the oligo dT substrate/probe comprises about 20 to about 30 thymidine residues. In some embodiments, the oligo dT substrate/probe comprises about 21 to about 30 thymidine residues. In some embodiments, the oligo dT substrate/probe comprises about 22 to about 30 thymidine residues. In some embodiments, the oligo dT substrate/probe comprises about 23 to about 30 thymidine residues. In some embodiments, the oligo dT substrate/probe comprises about 24 to about 30 thymidine residues. In some embodiments, the oligo dT substrate/probe comprises about 25 to about 30 thymidine residues.

In some embodiments, the oligo dT substrate/probe comprises about 15 to about 29 thymidine residues. In some embodiments, the oligo dT substrate/probe comprises about 15 to about 28 thymidine residues. In some embodiments, the oligo dT substrate/probe comprises about 15 to about 27 thymidine residues. In some embodiments, the oligo dT substrate/probe comprises about 15 to about 26 thymidine residues. In some embodiments, the oligo dT substrate/probe comprises about 15 to about 25 thymidine residues. In some embodiments, the oligo dT substrate/probe comprises about 15 to about 24 thymidine residues. In some embodiments, the oligo dT substrate/probe comprises about 15 to about 23 thymidine residues. In some embodiments, the oligo dT substrate/probe comprises about 15 to about 22 thymidine residues. In some embodiments, the oligo dT substrate/probe comprises about 15 to about 21 thymidine residues. In some embodiments, the oligo dT substrate/probe comprises about 15 to about 20 thymidine residues.

In some embodiments, the oligo dT substrate/probe comprises about 21 to about 29 thymidine residues. In some embodiments, the oligo dT substrate/probe comprises about 22 to about 28 thymidine residues. In some embodiments, the oligo dT substrate/probe comprises about 23 to about 27 thymidine residues. In some embodiments, the oligo dT substrate/probe comprises about 24 to about 26 thymidine residues.

Oligo dT substrates/probes may be of different lengths, e.g., may comprise at least about 15 thymidine residues, at least 16 about thymidine residues, at least about 17 thymidine residues, at least about 18 thymidine residues, at least about 19 thymidine residues, at least about 20 thymidine residues, at least about 21 thymidine residues, at least about 22 thymidine residues, at least about 23 thymidine residues, at least about 24 thymidine residues, at least about 25 thymidine residues, at least about 26 thymidine residues, at least about 27 thymidine residues, at least about 28 thymidine residues, at least about 29 thymidine residues, or at least about 30 thymidine residues.

In some embodiments, the oligo dT substrate/probe comprises about 15 thymidine residues. In some embodiments, the oligo dT substrate/probe comprises about 16 thymidine residues. In some embodiments, the oligo dT substrate/probe comprises about 17 thymidine residues. In some embodiments, the oligo dT substrate/probe comprises about 18 thymidine residues. In some embodiments, the oligo dT substrate/probe comprises about 19 thymidine residues. In some embodiments, the oligo dT substrate/probe comprises about 20 thymidine residues. In some embodiments, the oligo dT substrate/probe comprises about 21 thymidine residues. In some embodiments, the oligo dT substrate/probe comprises about 22 thymidine residues. In some embodiments, the oligo dT substrate/probe comprises about 23 thymidine residues. In some embodiments, the oligo dT substrate/probe comprises about 24 thymidine residues. In some embodiments, the oligo dT substrate/probe comprises about 25 thymidine residues. In some embodiments, the oligo dT substrate/probe comprises about 26 thymidine residues. In some embodiments, the oligo dT substrate/probe comprises about 27 thymidine residues. In some embodiments, the oligo dT substrate/probe comprises about 28 thymidine residues. In some embodiments, the oligo dT substrate/probe comprises about 29 thymidine residues. In some embodiments, the oligo dT substrate/probe comprises or about 30 thymidine residues. In a preferred embodiment, the oligo dT substrate/probe comprises 23 or 25 thymidine residues (SEQ ID NOS 16 and 15, respectively). In some embodiments, the oligo dT substrate/probe comprises 23 thymidine residues (SEQ ID NO:16). In some embodiments, the oligo dT substrate/probe comprises 25 thymidine residues (SEQ ID NO: 15).

In various embodiments, the methods described herein comprise an additional chromatography step to further remove dsRNA from the RNA preparation. In some embodiments, the method comprises contacting the sample containing single stranded RNA (e.g., capped polyadenylated mRNA) and dsRNA with an antibody, or antigen binding fragment thereof, that binds dsRNA; and separating dsRNA:antibody complex from the single-stranded RNA (e.g., capped polyadenylated mRNA).

In various embodiments, the dsRNA:antibody complex is separated from the single-stranded RNA by affinity chromatography. In some embodiments, the affinity chromatography comprises a 1 ml column. In some embodiments, the affinity chromatography comprises a 5 ml column. In some embodiments, the affinity chromatography comprises a 10 ml column.

Any resin/column that can bind an anti-dsRNA antibody may be used in the methods described herein to deplete antibody:dsRNA complexes and free antibody from the RNA sample. For example, protein A or protein G bound resins are most typically used to capture antibodies and antibody complexes. Protein A and protein G are immunoglobin-binding proteins originally isolated from bacteria. In preferred embodiments, the resin is a protein A-bound resin or bead. Protein A resins or beads are known in the art and commercially available, e.g., MabCapture™ A Select ProA™ resin).

In some embodiments, the antibody or antigen binding fragment thereof that binds dsRNA is selected from the group consisting of: a Camel Ig, a Llama Ig, an Alpaca Ig. Ig NAR, a Fab′ fragment, a F(ab′)2 fragment, a bispecific Fab dimer (Fab2), a trispecific Fab trimer (Fab3), an Fv, an single chain Fv protein (“scFv”), a bis-scFv, (scFv)2, a minibody, a diabody, a triabody, a tetrabody, a disulfide stabilized Fv protein (“dsFv”), and a single-domain antibody (sdAb, a camelid VHH, Nanobody). In some embodiments, the antibody or antigen binding fragment thereof that binds dsRNA is a monoclonal antibody. In some embodiments, the antibody is selected from the group consisting of: J2, J5, K1, K2, 1D3, CABT-B212, and 9D5. In particular embodiments, the antibody is J2.

Antibodies that bind to dsRNA are known and commercially available. Commercial vendors that sell one or more of the above listed dsRNA antibodies, include but are not limited to, MilliporeSigma, Jena Bioscience, Scicons, Thermo Fisher Scientific, Absolute Antibody, and Creative Diagnostics®.

In various embodiments, the RNA sample is contacted with at least about 1.5 mol %, at least about 2 mol %, at least about 2.5 mol %, at least about 3 mol %, at least about 3.5 mol %, at least about 4 mol %, at least about 4.5 mol %, at least about 5 mol %, at least about 5.5 mol %, at least about 6 mol %, at least about 6.5 mol %, at least about 7 mol %, at least about 7.5 mol %, at least about 15 mol %, at least about 30 mol %, or at least about 60 mol % antibody compared to total moles of RNA within the sample. For example, moles of RNA can be determined by dividing the total mass of RNA within the sample by the molecular weight (MW) of the RNA transcript. The molecular weight of the RNA transcript can be determined by its predicted length multiplied by 330 g/mol (average MW of an RNA nucleotide). RNA concentration can be determined by UV absorbance at 260 nm, which can then be multiplied by the volume to get the total mass of RNA in the sample. Moles of anti-dsRNA antibody can be similarly determined. If the concentration of anti-dsRNA antibody is not known, it can be determined by UV absorbance at 280 nm. If the concentration and volume of anti-dsRNA antibody is known, one can simply divide the total mass by the MW of the antibody to get the total moles of antibody. Once the total moles of RNA and moles anti-dsRNA antibody are determined, the appropriate percent of anti-dsRNA antibody can be added to the sample of RNA (e.g., 7.5 mol %, 15 mol %, 30 mol %, or 60 mol % anti-dsRNA antibody). For example, if there were 100 moles of RNA within a sample, one could add 60 moles of anti-dsRNA antibody to the RNA sample to obtain 60 mol % anti-dsRNA.

In some embodiments, the RNA sample is contacted with at least about 1.5 mol %, at least about 7.5% mol %, at least about 15% mol %, at least about 30% mol %, or at least about 60% mol % antibody, compared to total moles of RNA within the sample. In particular embodiments, the RNA sample is contacted with at least about 7.5 mol % antibody, compared to total moles of RNA within the sample.

In various embodiments, the RNA sample is contacted with about 1.5 mol %, about 2 mol %, about 2.5 mol %, about 3 mol %, about 3.5 mol %, about 4 mol %, about 4.5 mol %, about 5 mol %, about 5.5 mol %, about 6 mol %, about 6.5 mol %, about 7 mol %, about 7.5 mol %, about 15 mol %, about 30 mol %, or about 60 mol % antibody, compared to total moles of RNA within the sample. In particular embodiments, the RNA sample is contacted with about 7.5 mol % antibody, compared to total moles of RNA within the sample. In particular embodiments, the RNA sample is contacted with about 15 mol % antibody, compared to total moles of RNA within the sample. In particular embodiments, the RNA sample is contacted with about 30 mol % antibody, compared to total moles of RNA within the sample. In particular embodiments, the RNA sample is contacted with about 60 mol % antibody, compared to total moles of RNA within the sample.

In various embodiments, the RNA sample is contacted with about 1.5 mol % to about 60 mol % antibody, compared to total moles of RNA within the sample. In some embodiments, the RNA sample is contacted with about 2 mol % to about 60 mol % antibody, compared to total moles of RNA within the sample. In some embodiments, the RNA sample is contacted with about 2.5 mol % to about 60 mol % antibody, compared to total moles of RNA within the sample. In some embodiments, the RNA sample is contacted with about 3 mol % to about 60 mol % antibody, compared to total moles of RNA within the sample. In some embodiments, the RNA sample is contacted with about 3.5 mol % to about 60 mol % antibody, compared to total moles of RNA within the sample. In some embodiments, the RNA sample is contacted with about 4 mol % to about 60 mol % antibody, compared to total moles of RNA within the sample. In some embodiments, the RNA sample is contacted with about 4.5 mol % to about 60 mol % antibody, compared to total moles of RNA within the sample. In some embodiments, the RNA sample is contacted with about 5 mol % to about 60 mol % antibody, compared to total moles of RNA within the sample. In some embodiments, the RNA sample is contacted with about 5.5 mol % to about 60 mol % antibody, compared to total moles of RNA within the sample. In some embodiments, the RNA sample is contacted with about 6 mol % to about 60 mol % antibody, compared to total moles of RNA within the sample. In some embodiments, the RNA sample is contacted with about 6.5 mol % to about 60 mol % antibody, compared to total moles of RNA within the sample. In some embodiments, the RNA sample is contacted with about 7 mol % to about 60 mol % antibody, compared to total moles of RNA within the sample. In some embodiments, the RNA sample is contacted with about 7.5 mol % to about 60 mol % antibody, compared to total moles of RNA within the sample. In some embodiments, the RNA sample is contacted with about 15 mol % to about 60 mol % antibody, compared to total moles of RNA within the sample. In some embodiments, the RNA sample is contacted with about 30 mol % to about 60 mol % antibody, compared to total moles of RNA within the sample.

In various embodiments, the RNA sample is contacted with about 1.5 mol % to about 30 mol % antibody, compared to total moles of RNA within the sample. In some embodiments, the RNA sample is contacted with about 1.5 mol % to about 15 mol % antibody, compared to total moles of RNA within the sample. In some embodiments, the RNA sample is contacted with about 1.5 mol % to about 7.5 mol % antibody, compared to total moles of RNA within the sample. In some embodiments, the RNA sample is contacted with about 1.5 mol % to about 7 mol % antibody, compared to total moles of RNA within the sample. In some embodiments, the RNA sample is contacted with about 1.5 mol % to about 6.5 mol % antibody, compared to total moles of RNA within the sample. In some embodiments, the RNA sample is contacted with about 1.5 mol % to about 6 mol % antibody, compared to total moles of RNA within the sample. In some embodiments, the RNA sample is contacted with about 1.5 mol % to about 5.5 mol % antibody, compared to total moles of RNA within the sample. In some embodiments, the RNA sample is contacted with about 1.5 mol % to about 5 mol % antibody, compared to total moles of RNA within the sample. In some embodiments, the RNA sample is contacted with about 1.5 mol % to about 4.5 mol % antibody, compared to total moles of RNA within the sample. In some embodiments, the RNA sample is contacted with about 1.5 mol % to about 4 mol % antibody, compared to total moles of RNA within the sample. In some embodiments, the RNA sample is contacted with about 1.5 mol % to about 3.5 mol % antibody, compared to total moles of RNA within the sample. In some embodiments, the RNA sample is contacted with about 1.5 mol % to about 3 mol % antibody, compared to total moles of RNA within the sample. In some embodiments, the RNA sample is contacted with about 1.5 mol % to about 2.5 mol % antibody, compared to total moles of RNA within the sample. In some embodiments, the RNA sample is contacted with about 1.5 mol % to about 2 mol % antibody, compared to total moles of RNA within the sample.

### 5. Filtration

Impurities from RNA preparations may be filtered out during one or more steps in the methods described herein. For example, an RNA preparation may be passed through a membrane (e.g., an ultrafiltration membrane) to remove unwanted proteins (e.g., enzymes/proteins) from a previous reaction and/or increase the RNA concentration in the preparation.

As used herein, the terms “ultrafiltration” or “UF” refers to any technique in which a solution or a suspension is subjected to a semi-permeable membrane that retains macromolecules while allowing solvent and small solute molecules to pass through. The terms “ultrafiltration membrane” and “UF membrane” refer to a membrane that has pore sizes in the range of about 10 nanometer to about 100 nanometers (i.e., about 0.01 micrometers to about 0.1 micrometers). Ultrafiltration may be used to increase the concentration of RNA in a sample and/or remove impurities (e.g., proteins). RNA ultrafiltration techniques and methods are known in the art (see, e.g., Fernandez et al., “Cross flow filtration of RNA extracts by hollow fiber membrane,” Acta Biotechnol., 12:49-56, 1992).

Alternatively, diafiltration may be used to perform a buffer exchange and/or concentrate the RNA preparation. Generally, diafiltration is a technique that uses membranes to remove, replace, or lower the concentration of salts or solvents from solutions containing proteins, peptides, nucleic acids, and other biomolecules. Accordingly, as used herein, the term “diafiltration” or “DF” refers to a specialized class of filtration in which the retentate is diluted with solvent and re-filtered, to reduce the concentration of soluble permeate components. The term “retentate” refers to the portion of a sample/preparation or feed that has been retained by a membrane, and a retentate is the stream enriched in a retained species.

For example, in continuous diafiltration, a solvent is continuously added to the retentate at the same rate as the filtrate is generated. In this case, the retentate volume and the concentration of retained components does not change during the process. On the other hand, in discontinuous or sequential dilution diafiltration, an ultrafiltration step is followed by the addition of solvent to the retentate side; if the volume of solvent added to the retentate side is not equal or greater to the volume of filtrate generated, then the retained components will have a high concentration.

Diafiltration may be used to alter the pH, ionic strength, salt composition, buffer composition, or other properties of a solution or suspension of macromolecules.

As used herein, the terms “ultrafiltration/diafiltration” or “UF/DF” refer to any process, technique or combination of techniques that accomplishes ultrafiltration and/or diafiltration, either sequentially or simultaneously. UF/DF techniques, methods, and membranes are known in the art. See, e.g., Eon-Duval et al., Anal Biochem. 2003 May 1; 316(1):66-73.

In various embodiments the ultrafiltration, diafiltration, and/or UF/DF step utilizes tangential flow filtration (e.g., tangential flow ultrafiltration/diafiltration). Tangential flow filtration (TFF) is a process that uses membranes to separate components in a liquid solution or suspension (e.g., a feed sample) on the basis of size, molecular weight, or other differences. In these processes, the feed sample is pumped tangentially along the membrane surface and particles or molecules which are too large to pass through the membrane are retained and returned to a process tank for additional passes across the membrane (i.e., recirculation) until the feed sample is sufficiently clarified, concentrated, or purified. The cross-flow nature of TFF minimizes membrane fouling, thus permitting high volume processing per batch.

Membrane suitable for ultrafiltration and/or diafiltration may be made of a variety of different substrates or polymers known in the art. For example, in some embodiments, the TFF cassettes or hollow fiber cartridges comprise membrane(s) made of polysulfone, polyethersulfone, poly(methyl methacrylate), polyvinylidene fluoride, modified cellulose, regenerated cellulose, delta regenerated cellulose, cellulose acetate, and/or other polymers or substrates known to those skilled in the art. In some embodiments, the membrane is a polysulfone membrane. In some embodiments, the membrane is a polyethersulfone membrane. In some embodiments, the membrane is a poly(methyl methacrylate) membrane. In some embodiments, the membrane is a polyvinylidene fluoride membrane. In some embodiments, the membrane is a modified cellulose membrane. In some embodiments, the membrane is a regenerated cellulose membrane. In some embodiments, the membrane is a delta regenerated cellulose membrane. In some embodiments, the membrane is a cellulose acetate membrane. In preferred embodiments, the membrane is a hollow fiber membrane.

Exemplary TFF cassettes/membranes that are useful for the methods contemplated in particular embodiments herein include, but are not limited to, TFF cassettes supplied by MilliporeSigma Corporation (Burlington, Mass.), Pall Corporation (Port Washington, N.Y.), GE Healthcare Bio-Sciences (Piscataway, N.J.), and Sartorius AG (Bohemia, N.Y.) Exemplary MilliporeSigma Corporation TFF cassettes include, but are not limited to, Pellicon® cassettes (e.g., Pellicon® 2 cassettes, Pellicon® 2 Mini cassettes, Pellicon® 2 Maxi cassettes, Pellicon® 3 cassettes) with Biomax™ membrane, Ultracel™ membrane or Durapore® membrane. Exemplary Pall Corporation TFF cassettes include, but are not limited to Centrasette™ cassettes and Cadence™ single-use cassettes. Exemplary GE Healthcare Bio-Sciences TFF cassettes include, but are not limited to, Kvick™ Flow cassettes. Exemplary Sartorius AG cassettes include, but are not limited to, Hydrosart® cassettes.

In various embodiments, methods contemplated herein comprise additional filtration steps. In some embodiments, the filtration step comprises a final filter (i.e., the last filter in the method). In some embodiments, the filter is a sterilization filter. In some embodiments, the filter comprises a microfiltration membrane. In some embodiments, the filter comprises an ultrafiltration membrane. In some embodiments, the filter comprises a nanofiltration membrane. In some embodiments, the filter is a 0.22 μm filter.

### D. RNA and Related Therapeutic Genes and Proteins

Ribonucleic acid (RNA) is a nucleic acid molecule, i.e., a polymer consisting of nucleotide monomers. These nucleotides are usually adenosine-monophosphate (AMP), uridine-monophosphate (UMP), guanosine-monophosphate (GMP) and cytidine-monophosphate (CMP) monomers or analogs thereof, which are connected to each other via a molecular backbone. The backbone is formed by phosphodiester bonds between the sugar moieties, i.e., ribose, of each nucleotide monomer (base).

As used herein the term “nucleotide” refers to a heterocyclic nitrogenous base in N-glycosidic linkage with a phosphorylated sugar. Nucleotides are understood to include natural bases, and a wide variety of art-recognized modified bases. Such bases are generally located at the 1′ position of a nucleotide sugar moiety. Nucleotides generally comprise a base, sugar and a phosphate group. In ribonucleic acid (RNA), the sugar is a ribose, and in deoxyribonucleic acid (DNA) the sugar is a deoxyribose, i.e., a sugar lacking a hydroxyl group that is present in ribose. Exemplary natural nitrogenous bases include the purines, adenosine (A) and guanidine (G), and the pyrimidines, cytidine (C) and thymidine (T) (or in the context of RNA, uracil (U)). The C-1 atom of deoxyribose is bonded to N-1 of a pyrimidine or N-9 of a purine. Nucleotides are usually mono, di- or triphosphates. The nucleotides can be unmodified or modified at the sugar, phosphate and/or base moiety, (also referred to interchangeably as nucleotide analogs, nucleotide derivatives, modified nucleotides, non-natural nucleotides, and non-standard nucleotides; see for example, WO 92/07065 and WO 93/15187). Examples of modified nucleic acid bases are summarized by Limbach et al., (1994, Nucleic Acids Res. 22, 2183-2196).

A nucleotide may also be regarded as a phosphate ester of a nucleoside, with esterification occurring on the hydroxyl group attached to C-5 of the sugar. As used herein, the term “nucleoside” refers to a heterocyclic nitrogenous base in N-glycosidic linkage with a sugar. Nucleosides are recognized in the art to include natural bases, and also to include well known modified bases. Such bases are generally located at the 1′ position of a nucleoside sugar moiety. Nucleosides generally comprise a base and sugar group. The nucleosides can be unmodified or modified at the sugar, and/or base moiety, (also referred to interchangeably as nucleoside analogs, nucleoside derivatives, modified nucleosides, non-natural nucleosides, or non-standard nucleosides). As also noted above, examples of modified nucleic acid bases are summarized by Limbach et al., (1994, Nucleic Acids Res. 22, 2183-2196).

Messenger RNA (mRNA) is a single-stranded molecule of RNA that corresponds to the genetic sequence of a gene. mRNA can be obtainable by transcription of a DNA sequence, e.g., inside a cell. In a cell, transcription of DNA typically results in the generation of premature mRNA which is subsequently processed into mature mRNA. Processing of the premature RNA into mature messenger RNA usually comprises splicing, 5′-capping, polyadenylation, and export from the nucleus or the mitochondria. Alternatively, mRNA may be transcribed from recombinant DNA either in vitro (as described above) or in vivo. In this case, the translated recombinant DNA sequence typically does not comprise introns, thus splicing of exons is not required during processing.

The mature mRNA usually provides the nucleotide sequence that may be translated into an amino acid sequence of a particular peptide or protein. Typically, a mature mRNA comprises a 5′-cap, optionally a 5′UTR, an open reading frame, optionally a 3′UTR and a poly(A) sequence.

RNA (e.g., mRNA) useful in the methods/processes provided herein may be a product of DNA transcription (e.g., RNA transcript) or chemically synthesized. An RNA transcript (e.g., an in vitro transcribed mRNA) is the polynucleotide product of an in vitro transcription reaction.

As used herein, an “RNA transcript” refers to a ribonucleic acid produced by an in vitro transcription reaction using a DNA template and an RNA polymerase. An RNA transcript typically includes the coding sequence for a gene of interest and a poly(A) tail. The RNA transcript can include modifications, e.g., modified nucleotides. As used herein, the term RNA transcript includes and is interchangeable with mRNA, whether transcribed from a DNA template or chemically synthesized.

RNA transcripts and mRNA are typically single-stranded (ssRNA), however double stranded RNA (dsRNA) is a common byproduct of transcription (e.g., in vitro transcription). It is hypothesized that dsRNA can occur in multiple ways, including but not limited to, turn-around transcription (cis), random priming of abortive transcripts (cis/trans), and/or anti-sense transcription of the DNA. Notwithstanding the mechanism of dsRNA formation, it is known that dsRNA is typically toxic to cells, e.g., when dsRNA is administered in vivo, recipient cells may sense it as an invading virus, which can trigger an immune response.

The RNA transcript (e.g., mRNA sample) to be purified in the methods/processes described herein is not limited by the source of RNA. In some embodiments, the RNA is synthesized by in vitro transcription of a DNA template comprising a gene cloned in a linearized or linear plasmid vector, or by in vitro transcription of a DNA template that is synthesized by PCR or RT-PCR (i.e., by IVT of a PCR amplification product). The RNA may be capped as describe above. In one embodiment the RNA transcript includes a 5′ cap, typically added post transcriptionally.

In certain embodiments, the RNA is polyadenylated (poly(A)). The poly(A) may be encoded into the DNA template or added after transcription. In particular embodiments, an RNA contemplated herein comprises a poly(A) tail to help protect the RNA from exonuclease degradation, stabilize the RNA, and facilitate translation. In certain embodiments, an RNA comprises a 3′ poly(A) tail structure. Methods for polyadenylating RNA are known in the art (P L Wigley et al. Mol Cell Biol. 1990 April; 10(4): 1705-1713; and Wakiyama et al., Biochimie. 1997 December; 79(12):781-5)

In particular embodiments, the length of the poly(A) tail is at least about 10, 25, 50, 75, 100, 150, 200, 250, 300, 350, 400, 450, or at least about 500 or more adenine nucleotides or any intervening number of adenine nucleotides. In particular embodiments, the length of the poly(A) tail is at least about 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 202, 203, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, or 275 or more adenine nucleotides.

In particular embodiments, the length of the poly(A) tail is about 10 to about 500 adenine nucleotides, about 50 to about 500 adenine nucleotides, about 100 to about 500 adenine nucleotides, about 150 to about 500 adenine nucleotides, about 200 to about 500 adenine nucleotides, about 250 to about 500 adenine nucleotides, about 300 to about 500 adenine nucleotides, about 50 to about 450 adenine nucleotides, about 50 to about 400 adenine nucleotides, about 50 to about 350 adenine nucleotides, about 100 to about 500 adenine nucleotides, about 100 to about 450 adenine nucleotides, about 100 to about 400 adenine nucleotides, about 100 to about 350 adenine nucleotides, about 100 to about 300 adenine nucleotides, about 150 to about 500 adenine nucleotides, about 150 to about 450 adenine nucleotides, about 150 to about 400 adenine nucleotides, about 150 to about 350 adenine nucleotides, about 150 to about 300 adenine nucleotides, about 150 to about 250 adenine nucleotides, about 150 to about 200 adenine nucleotides, about 200 to about 500 adenine nucleotides, about 200 to about 450 adenine nucleotides, about 200 to about 400 adenine nucleotides, about 200 to about 350 adenine nucleotides, about 200 to about 300 adenine nucleotides, about 250 to about 500 adenine nucleotides, about 250 to about 450 adenine nucleotides, about 250 to about 400 adenine nucleotides, about 250 to about 350 adenine nucleotides, or about 250 to about 300 adenine nucleotides or any intervening range of adenine nucleotides.

In some embodiments, the RNA transcript includes a 5′UTR and a 3′UTR.

Terms that describe the orientation of polynucleotides (e.g., RNA transcripts or mRNA) include: 5′ (normally the end of the polynucleotide having a free phosphate group) and 3′ (normally the end of the polynucleotide having a free hydroxyl (OH) group). Polynucleotide sequences can be annotated in the 5′ to 3′ orientation or the 3′ to 5′ orientation. For DNA and mRNA, the 5′ to 3′ strand is designated the “sense,” “plus,” or “coding” strand because its sequence is identical to the sequence of the pre-messenger (pre-mRNA) [except for uracil (U) in RNA, instead of thymine (T) in DNA]. For DNA and mRNA, the complementary 3′ to 5′ strand which is the strand transcribed by the RNA polymerase is designated as “template,” “antisense,” “minus,” or “non-coding” strand. As used herein, the term “reverse orientation” refers to a 5′ to 3′ sequence written in the 3′ to 5′ orientation or a 3′ to 5′ sequence written in the 5′ to 3″ orientation.

The terms “complementary” and “complementarity” refer to polynucleotides (i.e., a sequence of nucleotides) related by the base-pairing rules. For example, the complementary strand of the DNA sequence 5′ A G T C A T G 3′ is 3′T C A G T A C 5′. The latter sequence is often written as the reverse complement with the 5′ end on the left and the 3′ end on the right, 5′ C A T G A C T 3′. A sequence that is equal to its reverse complement is said to be a palindromic sequence. Complementarity can be “partial,” in which only some of the nucleic acids' bases are matched according to the base pairing rules or there can be “complete” or “total” complementarity between the nucleic acids.

Moreover, it will be appreciated by those of ordinary skill in the art that, as a result of the degeneracy of the genetic code, there are many nucleotide sequences that may encode a polypeptide, or fragment of variant thereof, as contemplated herein. Some of these polynucleotides bear minimal homology to the nucleotide sequence of any native gene. Nonetheless, polynucleotides that vary due to differences in codon usage are specifically contemplated in particular embodiments, for example polynucleotides that are optimized for human and/or primate codon selection. In one embodiment, polynucleotides comprising particular allelic sequences are provided. Alleles are endogenous polynucleotide sequences that are altered as a result of one or more mutations, such as deletions, additions and/or substitutions of nucleotides.

The RNA transcript may be a coding RNA (e.g., mRNA), which encodes a protein or a fragment or variant thereof, including but not limited to secreted proteins, plasma membrane proteins, cytoplasmic or cytoskeletal proteins, intracellular membrane bound proteins, proteins associated with human disease, targeting moieties, fusion proteins, enzyme, endonucleases, exonucleases, CRISPR-associated nuclease (e.g., Cas9 and variants thereof), meganuclease or homing endonuclease (HE), transcription activator-like effector nuclease (TALEN), megaTAL, zinc finger nuclease, tumor antigens, pathogenic antigens, allergenic antigens, autoimmune antigens, or those proteins encoded by the human genome. RNA sequences that code for a peptide or a protein may be readily identified by one of skill in the art by using public and private databases, e.g., NCBI GenBank or PubMed. In particular embodiments, the coding RNAs may be, e.g., mRNAs, viral RNAs, or replicon RNAs.

In various embodiments, the RNA transcript or mRNA codes for a nuclease (e.g., an endonuclease or exonuclease). The term “endonuclease” refers to enzymes that cleave the phosphodiester bond within a polynucleotide chain. The polynucleotide may be double-stranded DNA (dsDNA), single-stranded DNA (ssDNA), RNA, double-stranded hybrids of DNA and RNA, and synthetic DNA (for example, containing bases other than A, C, G, and T). An endonuclease may cut a polynucleotide symmetrically, leaving “blunt” ends, or in positions that are not directly opposing, creating overhangs, which may be referred to as “sticky ends.” The methods and compositions described herein may be applied to cleavage sites generated by endonucleases. Endonucleases include, but are not limited to, gene editing enzymes such as meganucleases, homing endonucleases (HEs), megaTALs, TALENs, zinc finger nucleases, CRISPR-associated nucleases, or functional variants thereof.

In various embodiments, the RNA transcript or mRNA codes of a gene editing endonuclease. In some embodiments, the gene editing endonuclease is a meganuclease, homing endonuclease (HE), megaTAL, TALEN, zinc finger nuclease, or CRISPR-associated nuclease (e.g., Cas9). In particular embodiments, the gene editing endonuclease is a meganuclease, homing endonuclease (HE), or megaTAL.

The terms “homing endonuclease” and “meganuclease” are used interchangeably and refer to naturally-occurring nucleases that recognize 12-45 base-pair cleavage sites (e.g., a target site) and are commonly grouped into five families based on sequence and structure motifs: LAGLIDADG (SEQ ID NO: 14), GIY-YIG, HNH, His-Cys box, and PD-(D/E)XK. See, e.g., Stoddard Structure. 2011 Jan. 12; 19(1):7-15.

A “megaTAL” refers to a polypeptide comprising a TALE DNA-binding domain and a homing endonuclease variant that binds and cleaves a DNA target sequence in a target gene. See, e.g., Boissel et al. Methods Mol Biol (2015); 1239:171-96. In some embodiments, the megaTAL further comprises one or more linkers and/or additional functional domains, e.g., an end-processing enzymatic domain of an end-processing enzyme that exhibits 5′-3′ exonuclease, 5′-3′ alkaline exonuclease, 3′-5′ exonuclease (e.g., Trex2, ExoI or ExoX), 5′ flap endonuclease, helicase or template-independent DNA polymerases activity.

The term “clustered regularly interspaced short palindromic repeats” or “CRISPR” refers to a family of DNA sequences originally found in the genomes of prokaryotic organisms such as bacteria and archaea and are used to detect and destroy DNA from bacteriophages. CRISPR sequences in combination with a nuclease (e.g., a Cas nuclease; CRISPR-Cas) can also be used to edit genes within organisms and have a variety of applications in research, gene editing, and therapeutics. See, e.g., Nature Biotechnology volume 38, pages 824-844 (2020).

The terms “CRISPR-associate nuclease” and “Cas nuclease” are used interchangeably and refer to an RNA guided sequence-specific nuclease that uses CRISPR sequences as a guide to generate specific single or double stranded breaks in DNA. Generally, targeting by CRISPR-Cas systems requires a short sequence known as a protospacer-adjacent motif (PAM) occur near the target DNA site.

The term “zinc-finger nuclease” (ZFN) refers to artificial restriction enzymes generated by fusing a zinc finger DNA-binding domain to a DNA-cleavage domain. Zinc finger domains can be engineered to bind to a desired target site. In some embodiments, the cleavage domain comprises the non-specific cleavage domain of Fokl. In other embodiments, the cleavage domain comprises all or an active portion of another nuclease.

The term “TAL effector nuclease” (TALEN) refers to a nuclease comprising a TAL-effector domain (TALE) fused to a nuclease domain. TAL-effector DNA binding domains, isolated from the plant pathogen Xanthomonas have been described (see Boch et al., (2009) Science 29 Oct. 2009 (10.1126/science.117881) and Moscou and Bogdanove, (2009) Science 29 Oct. 2009 (10.1126/science.1178817)). These DNA binding domains may be engineered to bind to a desired target and fused to a nuclease domain, such as the Fokl nuclease domain, to derive a TAL effector domain-nuclease fusion protein.

A “target site” or “target sequence” is a chromosomal or extrachromosomal nucleic acid sequence that defines a portion of a nucleic acid to which a binding molecule will bind and/or cleave, provided sufficient conditions for binding and/or cleavage exist. When referring to a polynucleotide sequence or SEQ ID NO. that references only one strand of a target site or target sequence, it would be understood that the target site or target sequence bound and/or cleaved by a nuclease variant is double-stranded and comprises the reference sequence and its complement. In various embodiments, the target site is in an immune system checkpoint gene, globin gene, gene that encodes a polypeptide that contributes to repression of γ-globin gene expression and/or HbF, or immunosuppressive signaling gene.

In various embodiments, the nuclease (e.g., endonuclease, HE, megaTAL, TALEN, ZFN, or CRISPR-Cas) target site is within an immune system checkpoint gene, globin gene, gene that encodes a polypeptide that contributes to repression of γ-globin gene expression and HbF, or immunosuppressive signaling gene. In some embodiments, the target site is within a gene selected from the group consisting of: programmed cell death protein 1 (PD-1; PDCD1), lymphocyte activation gene 3 protein (LAG-3), T cell immunoglobulin domain and mucin domain protein 3 (TIM-3), cytotoxic T lymphocyte antigen-4 (CTLA-4), band T lymphocyte attenuator (BTLA), T cell immunoglobulin and immunoreceptor tyrosine-based inhibitory motif domain (TIGIT), V-domain Ig suppressor of T cell activation (VISTA), and killer cell immunoglobulin-like receptor (KIR), CCR5, TRAC (TCRα), TCRβ, IL10Rα, IL10Rβ, TGFBR1, TGFBR2, CBL-B, PCSK9, AHR, BTK, α-globin, β-globin, γ-globin, and BCL11A gene.

In some embodiments, the target site is a sequence in the human TRAC gene. In some embodiments, the target site is a sequence in the PD1 gene. In some embodiments, the target site is a sequence in the PCSK9 gene. In some embodiments, the target site is a sequence in BCL11A. In some embodiments, the target site is a sequence in BCL11A.

Other target genes may include, but are not limited to, α-globin, β-globin, γ-globin, BCL11A, KLF1, SOX6, GATA1, LSD1, alpha folate receptor (FRα), αvβ6 integrin, B cell maturation antigen (BCMA), B7-H3 (CD276), B7-H6, carbonic anhydrase IX (CAIX), CD16, CD19, CD20, CD22, CD30, CD33, CD37, CD38, CD44, CD44v6, CD44v7/8, CD70, CD79a, CD79b, CD123, CD133, CD138, CD171, carcinoembryonic antigen (CEA), C-type lectin-like molecule-1 (CLL-1), CD2 subset 1 (CS-1), chondroitin sulfate proteoglycan 4 (CSPG4), cutaneous T cell lymphoma-associated antigen 1 (CTAGE1), epidermal growth factor receptor (EGFR), epidermal growth factor receptor variant III (EGFRvIII), epithelial glycoprotein 2 (EGP2), epithelial glycoprotein 40 (EGP40), epithelial cell adhesion molecule (EPCAM), ephrin type-A receptor 2 (EPHA2), fibroblast activation protein (FAP), Fc Receptor Like 5 (FCRL5), fetal acetylcholinesterase receptor (AchR), ganglioside G2 (GD2), ganglioside G3 (GD3), Glypican-3 (GPC3), EGFR family including ErbB2 (HER2), IL-11Rα, IL-13Rα2, Kappa, cancer/testis antigen 2 (LAGE-IA), Lambda, Lewis-Y (LeY), LI cell adhesion molecule (LI-CAM), melanoma antigen gene (MAGE)-A1, MAGE-A3, MAGE-A4, MAGE-A6, MAGEA10, melanoma antigen recognized by T cells 1 (MelanA or MARTI), Mesothelin (MSLN), MUC1, MUC16, MHC class I chain related proteins A (MICA), MHC class I chain related proteins B (MICB), neural cell adhesion molecule (NCAM), cancer/testis antigen 1 (NY-ESO-1), polysialic acid; placenta-specific 1 (PLAC1), preferentially expressed antigen in melanoma (PRAME), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), tumor endothelial marker 1 (TEMI/CD248), tumor endothelial marker 7-related (TEM7R), TEM5, TEM8, trophoblast glycoprotein (TPBG), UL16-binding protein (ULBP) 1, ULBP2, ULBP3, ULBP4, ULBP5, ULBP6, vascular endothelial growth factor receptor 2 (VEGFR2), Wilms tumor 1 (WT-1) gene, and Wiskott-Aldrich syndrome (WAS) gene.

In some embodiments, the nuclease (e.g., endonuclease, HE, megaTAL, TALEN, ZFN, or CRISPR-Cas) target site is within a gene selected from the group consisting of: programmed cell death protein 1 (PD-1; PDCD1), lymphocyte activation gene 3 protein (LAG-3), T cell immunoglobulin domain and mucin domain protein 3 (TIM-3), cytotoxic T lymphocyte antigen-4 (CTLA-4), band T lymphocyte attenuator (BTLA), T cell immunoglobulin and immunoreceptor tyrosine-based inhibitory motif domain (TIGIT), V-domain Ig suppressor of T cell activation (VISTA), and killer cell immunoglobulin-like receptor (KIR), CCR5, TRAC (TCRα), IL10Rα, TGFBR2, CBL-B, PCSK9, AHR, BTK, α-globin, β-globin, γ-globin, and BCL11A gene.

In various embodiments, the nuclease (e.g., endonuclease, HE, megaTAL, TALEN, ZFN, or CRISPR-Cas) target site is within a TRAC (TCRα) gene, a PDCD1 (PD-1) gene, or a PCSK9 gene. In particular embodiments, a TCRα megaTAL RNA comprises the sequence set forth in SEQ ID NO: 2 or 3 (see, e.g., WO 2018/071565, which is incorporated herein by reference in its entirety). In particular embodiments, a PD-1 megaTAL RNA comprises the sequence set forth in SEQ ID NO: 5 or 6 (see, e.g., WO 2018/049226, which is incorporated herein by reference in its entirety). In particular embodiments, a PCSK9 megaTAL RNA comprises the sequence set forth in SEQ ID NO: 8 or 9 (see, e.g., WO 2019/070974, which is incorporated herein by reference in its entirety).

In various embodiments, the RNA transcript encodes for an exonuclease, endprocessing enzyme, or fragment or variant thereof. In some embodiments, the RNA transcript is an exonuclease, endprocessing enzyme, or fragment or variant thereof, selected from the group consisting of: Trex2, Trex1, Trex1 without transmembrane domain, Apollo, Artemis, DNA2, ExoI, ExoT, ExoIII, ExoX, Fen1, Fan1, MreII, Rad2, Rad9, TdT (terminal deoxynucleotidyl transferase), PNKP, RecE, RecJ, RecQ, Lambda exonuclease, Sox, Vaccinia DNA polymerase, exonuclease I, exonuclease III, exonuclease VII, NDK1, NDK5, NDK7, NDK8, WRN, T7-exonuclease Gene 6, avian myeloblastosis virus integration protein (IN), Bloom, Antarctic Phosphatase, Alkaline Phosphatase, Poly nucleotide Kinase (PNK), ApeI, Mung Bean nuclease, Hex1, TTRAP (TDP2), Sgs1, Sae2, CUP, Pol mu, Pol lambda, MUS81, EME1, EME2, SLX1, SLX4 and UL-12. In some embodiments, the exonuclease is Trex2, or biologically active fragment thereof. In particular embodiments, the Trex2 RNA comprises the sequence set forth in SEQ ID NO: 11 or 12.

In various embodiments, the RNA transcript may code for a protein or polypeptide associated with a disease (e.g., a therapeutically active protein or polypeptide). In some embodiments, the therapeutically active protein or polypeptide is α-globin, β-globin, γ-globin, FVIII or anti-hemophilic factor (AHF), ATP-binding cassette sub-family D member 1 (ABCD1), adenosine deaminase, interleukin 2 receptor gamma, tripeptidyl peptidase 1, alpha-L iduronidase, iduronate 2-sulfatase.

Alternatively, the selected RNA sequence may be any RNA as defined herein, particularly a messenger RNA (mRNA), small interfering RNA (siRNA), an antisense RNA, a CRISPR RNA, a circular RNA (circRNA), a ribozyme, an aptamer, a riboswitch, an immunostimulating RNA, a transfer RNA (tRNA), a ribosomal RNA (rRNA), a small nuclear RNA (snRNA), a small nucleolar RNA (snoRNA), a microRNA (miRNA), or a Piwi-interacting RNA (piRNA). In some embodiments, the RNA may comprise naturally occurring and/or modified nucleotides.

In one embodiment, an RNA (e.g., mRNA) comprises one or more modified nucleosides selected from the group consisting of: pseudouridine, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxy methyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxy methylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonylcarbamoyladenosine, N6,N6-dimethyladenosine, 7-methyladenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In one embodiment, an RNA (e.g., mRNA) comprises one or more modified nucleosides selected from the group consisting of: pseudouridine, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxy methyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine.

In one embodiment, an RNA (e.g., mRNA) comprises one or more modified nucleosides selected from the group consisting of: 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In one embodiment, an RNA (e.g., mRNA) comprises one or more modified nucleosides selected from the group consisting of: 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In one embodiment, an RNA (e.g., mRNA) comprises one or more modified nucleosides selected from the group consisting of: inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In one embodiment, an RNA (e.g., mRNA) comprises one or more pseudouridines, one or more 5-methyl-cytosines, and/or one or more 5-methyl-cytidines. In one embodiment, an mRNA comprises one or more pseudouridines. In one embodiment, an mRNA comprises one or more 5-methyl-cytidines. In one embodiment, an mRNA comprises one or more 5-methyl-cytosines.

### E. Sequence Listing

| SEQ | | |
|---|---|---|
| ID | | |
| NO: | Description | Sequence |
| 1 | TCRα | CCTCCGAAGAAGAAGCGGAAAGTCGTGGAC |
| | megaTAL | CTCCGGACCCTGGGTTACTCTCAGCAGCAG |
| | DNA | CAGGAGAAGATCAAGCCGAAGGTGCGGTCG |
| | | ACTGTGGCCCAGCATCACGAGGCCCTGGTG |
| | | GGACACGGCTTCACCCACGCCCACATTGTG |
| | | GCCCTGAGCCAGCACCCGGCAGCGCTGGGA |
| | | ACCGTGGCCGTGACCTACCAGCACATCATT |
| | | ACTGCCCTGCCTGAAGCGACCCACGAGGAT |
| | | ATCGTGGGCGTCGGAAAGCAGTGGTCCGGA |
| | | GCCAGAGCCTTGGAGGCTCTGCTGACTGAC |
| | | GCCGGAGAGCTGCGGGGCCCGCCCCTGCAA |
| | | CTGGATACCGGCCAGCTCGTGAAAATCGCC |
| | | AAGAGAGGAGGAGTGACCGCCATGGAAGCC |
| | | GTGCATGCATCCCGCAATGCACTGACTGGT |
| | | GCACCCCTGAACCTCACTCCTGACCAGGTC |
| | | GTCGCTATCGCAAGCAACATCGGAGGGAAA |
| | | CAAGCTCTCGAGACAGTGCAGCGCCTCCTG |
| | | CCAGTGCTTTGCCAGGACCACGGCCTGACT |
| | | CCAGACCAGGTGGTCGCTATTGCGTCGAAC |
| | | ATTGGAGGGAAGCAAGCCCTTGAAACCGTG |
| | | CAGAGGCTGCTCCCGGTGCTGTGCCAAGAC |
| | | CATGGACTCACCCCGGACCAAGTGGTGGCT |
| | | ATTGCTAGCAACATTGGCGGTAAGCAGGCG |
| | | CTGGAGACAGTCCAGCGGCTGCTGCCGGTG |
| | | TTGTGCCAAGATCACGGTCTTACCCCAGAC |
| | | CAAGTCGTGGCGATTGCCTCCAACGGTGGC |
| | | GGCAAGCAAGCACTCGAAACTGTCCAGAGA |
| | | CTGCTCCCTGTGCTCTGTCAAGACCACGGG |
| | | TTGACCCCCGACCAAGTGGTGGCCATCGCC |
| | | TCCCATGATGGAGGAAAGCAGGCCCTCGAG |
| | | ACTGTCCAGCGACTGCTCCCCGTGTTGTGT |
| | | CAGGATCATGGATTGACGCCCGATCAGGTC |
| | | GTGGCCATTGCCTCCCACGACGGTGGAAAG |
| | | CAAGCGCTGGAAACTGTGCAGCGGTTGCTG |
| | | CCGGTCCTGTGCCAGGACCACGGACTGACT |
| | | CCGGACCAGGTGGTCGCCATCGCATCCAAC |
| | | ATTGGTGGCAAGCAGGCTCTCGAAACCGTC |
| | | CAACGCCTGTTGCCGGTGCTGTGTCAGGAT |
| | | CATGGACTGACCCCGGACCAAGTGGTGGCT |
| | | ATCGCCTCCAACAACGGGGGCAAACAGGCC |
| | | CTGGAAACCGTGCAACGCCTGCTGCCCGTC |
| | | CTCTGCCAGGACCACGGTCTGACCCCTGAC |
| | | CAGGTCGTCGCGATCGCGTCAAATGGGGGG |
| | | GGCAAACAGGCTCTGGAAACGGTGCAGCGG |
| | | CTCCTTCCGGTGTTATGCCAGGACCACGGG |
| | | CTGACTCCCGACCAGGTGGTGGCGATCGCC |
| | | TCGAACAACGGAGGCAAACAAGCCCTGGAG |
| | | ACTGTGCAGAGACTCCTGCCCGTGCTGTGC |
| | | CAAGACCATGGGCTCACCCCTGATCAGGTG |
| | | GTGGCAATCGCCTCAAACATCGGCGGGAAG |
| | | CAGGCACTGGAAACTGTGCAGAGACTCCTG |
| | | CCCGTGCTGTGCCAAGACCATGGGCTGACC |
| | | CCGGACCAAGTGGTGGCTATCGCCTCCCAC |
| | | GACGGGGGCAAACAGGCCCTGGAATCCATC |
| | | GTCGCTCAGCTGAGCCGGCCTGACCCAGCA |
| | | CTCGCCGCCCTGACCAATGACCATCTGGTC |
| | | GCCCTGGCCTGCCTGGGAGGCAGACCCGCG |
| | | ATGGACGCGGTCAAGAAGGGTCTGCCGCAC |
| | | GCCCCTGAGCTTATTCGGAGAGTGAACAGG |
| | | CGCATCGGTGAACGCACCTCCCATCGGGTC |
| | | GCAATCTCTAGAGTGGGCGGATCGTCCCGG |
| | | CGGGAGTCCATCAATCCTTGGATCCTGACC |
| | | GGCTTCGCCGACGCCGAAGGCTCCTTCATC |
| | | CTGGACATCAGGAACAGGAACAACGAGTCA |
| | | AACAGGTACCGCACCTCCCTTCGGTTCCAG |
| | | ATTACTCTGCACAACAAGGATAAGTCCATC |
| | | CTCGAGAACATCCAGTCAACCTGGAAAGTG |
| | | GGCAAGATCACTAACTCCTCGGACCGCGCA |
| | | GTGATGCTCCGGGTGACCCGCTTCGAGGAC |
| | | CTGAAGGTGATCATTGACCACTTCGAGAAG |
| | | TACCCTCTCATAACCCAGAAGCTGGGAGAT |
| | | TACAAGCTGTTTAAGCAGGCGTTCTCCGTG |
| | | ATGGAGAACAAAGAACACCTTAAGGAGAAT |
| | | GGGATTAAGGAACTGGTCCGCATTAAGGCC |
| | | AAGATGAACTGGGGACTGAACGACGAGTTG |
| | | AAAAAGGCATTTCCTGAAAACATCTCCAAG |
| | | GAACGGCCGCTCATCAACAAGAACATTCCC |
| | | AATTTCAAGTGGCTGGCGGGGTTCACTGCC |
| | | GGGGACGGACACTTCGGAGTGAACCTGAAG |
| | | AAGGTGAAGGGCACCGCCAAGGTGTACGTG |
| | | GGCCTGCGGTTCGCGATCAGCCAGCACATC |
| | | CGGGATAAGAACCTGATGAACAGCCTCATC |
| | | ACCTACCTGGGATGCGGAAGCATCCGGGAG |
| | | AAGAACAAGTCAGAATTCCGATGGCTGGAA |
| | | TTTGAAGTGACCAAGTTCTCCGACATCAAC |
| | | GACAAGATCATCCCCGTGTTCCAGGAGAAC |
| | | ACCCTCATTGGAGTGAAGCTGGAGGACTTC |
| | | GAGGACTGGTGCAAGGTGGCCAAGCTCATC |
| | | GAAGAGAAGAAGCACCTGACCGAAAGCGGC |
| | | CTGGATGAGATTAAGAAGATTAAGCTCAAC |
| | | ATGAACAAGGGAAGATAGTAG |
| 2 | TCRα | GGGGCCGCCACCAUGGGAUCCGCCCCUCCG |
| | megaTAL | AAGAAGAAGCGGAAAGUCGUGGACCUCCGG |
| | RNA | ACCCUGGGUUACUCUCAGCAGCAGCAGGAG |
| | | AAGAUCAAGCCGAAGGUGCGGUCGACUGUG |
| | | GCCCAGCAUCACGAGGCCCUGGUGGGACAC |
| | | GGCUUCACCCACGCCCACAUUGUGGCCCUG |
| | | AGCCAGCACCCGGCAGCGCUGGGAACCGUG |
| | | GCCGUGACCUACCAGCACAUCAUUACUGCC |
| | | CUGCCUGAAGCGACCCACGAGGAUAUCGUG |
| | | GGCGUCGGAAAGCAGUGGUCCGGAGCCAGA |
| | | GCCUUGGAGGCUCUGCUGACUGACGCCGGA |
| | | GAGCUGCGGGGCCCGCCCCUGCAACUGGAU |
| | | ACCGGCCAGCUCGUGAAAAUCGCCAAGAGA |
| | | GGAGGAGUGACCGCCAUGGAAGCCGUGCAU |
| | | GCAUCCCGCAAUGCACUGACUGGUGCACCC |
| | | CUGAACCUCACUCCUGACCAGGUCGUCGCU |
| | | AUCGCAAGCAACAUCGGAGGGAAACAAGCU |
| | | CUCGAGACAGUGCAGCGCCUCCUGCCAGUG |
| | | CUUUGCCAGGACCACGGCCUGACUCCAGAC |
| | | CAGGUGGUCGCUAUUGCGUCGAACAUUGGA |
| | | GGGAAGCAAGCCCUUGAAACCGUGCAGAGG |
| | | CUGCUCCCGGUGCUGUGCCAAGACCAUGGA |
| | | CUCACCCCGGACCAAGUGGUGGCUAUUGCU |
| | | AGCAACAUUGGCGGUAAGCAGGCGCUGGAG |
| | | ACAGUCCAGCGGCUGCUGCCGGUGUUGUGC |
| | | CAAGAUCACGGUCUUACCCCAGACCAAGUC |
| | | GUGGCGAUUGCCUCCAACGGUGGCGGCAAG |
| | | CAAGCACUCGAAACUGUCCAGAGACUGCUC |
| | | CCUGUGCUCUGUCAAGACCACGGGUUGACC |
| | | CCCGACCAAGUGGUGGCCAUCGCCUCCCAU |
| | | GAUGGAGGAAAGCAGGCCCUCGAGACUGUC |
| | | CAGCGACUGCUCCCCGUGUUGUGUCAGGAU |
| | | CAUGGAUUGACGCCCGAUCAGGUCGUGGCC |
| | | AUUGCCUCCCACGACGGUGGAAAGCAAGCG |
| | | CUGGAAACUGUGCAGCGGUUGCUGCCGGUC |
| | | CUGUGCCAGGACCACGGACUGACUCCGGAC |
| | | CAGGUGGUCGCCAUCGCAUCCAACAUUGGU |
| | | GGCAAGCAGGCUCUCGAAACCGUCCAACGC |
| | | CUGUUGCCGGUGCUGUGUCAGGAUCAUGGA |
| | | CUGACCCCGGACCAAGUGGUGGCUAUCGCC |
| | | UCCAACAACGGGGGCAAACAGGCCCUGGAA |
| | | ACCGUGCAACGCCUGCUGCCCGUCCUCUGC |
| | | CAGGACCACGGUCUGACCCCUGACCAGGUC |
| | | GUCGCGAUCGCGUCAAAUGGGGGGGGCAAA |
| | | CAGGCUCUGGAAACGGUGCAGCGGCUCCUU |
| | | CCGGUGUUAUGCCAGGACCACGGGCUGACU |
| | | CCCGACCAGGUGGUGGCGAUCGCCUCGAAC |
| | | AACGGAGGCAAACAAGCCCUGGAGACUGUG |
| | | CAGAGACUCCUGCCCGUGCUGUGCCAAGAC |
| | | CAUGGGCUCACCCCUGAUCAGGUGGUGGCA |
| | | AUCGCCUCAAACAUCGGCGGGAAGCAGGCA |
| | | CUGGAAACUGUGCAGAGACUCCUGCCCGUG |
| | | CUGUGCCAAGACCAUGGGCUGACCCCGGAC |
| | | CAAGUGGUGGCUAUCGCCUCCCACGACGGG |
| | | GGCAAACAGGCCCUGGAAUCCAUCGUCGCU |
| | | CAGCUGAGCCGGCCUGACCCAGCACUCGCC |
| | | GCCCUGACCAAUGACCAUCUGGUCGCCCUG |
| | | GCCUGCCUGGGAGGCAGACCCGCGAUGGAC |
| | | GCGGUCAAGAAGGGUCUGCCGCACGCCCCU |
| | | GAGCUUAUUCGGAGAGUGAACAGGCGCAUC |
| | | GGUGAACGCACCUCCCAUCGGGUCGCAAUC |
| | | UCUAGAGUGGGCGGAUCGUCCCGGCGGGAG |
| | | UCCAUCAAUCCUUGGAUCCUGACCGGCUUC |
| | | GCCGACGCCGAAGGCUCCUUCAUCCUGGAC |
| | | AUCAGGAACAGGAACAACGAGUCAAACAGG |
| | | UACCGCACCUCCCUUCGGUUCCAGAUUACU |
| | | CUGCACAACAAGGAUAAGUCCAUCCUCGAG |
| | | AACAUCCAGUCAACCUGGAAAGUGGGCAAG |
| | | AUCACUAACUCCUCGGACCGCGCAGUGAUG |
| | | CUCCGGGUGACCCGCUUCGAGGACCUGAAG |
| | | GUGAUCAUUGACCACUUCGAGAAGUACCCU |
| | | CUCAUAACCCAGAAGCUGGGAGAUUACAAG |
| | | CUGUUUAAGCAGGCGUUCUCCGUGAUGGAG |
| | | AACAAAGAACACCUUAAGGAGAAUGGGAUU |
| | | AAGGAACUGGUCCGCAUUAAGGCCAAGAUG |
| | | AACUGGGGACUGAACGACGAGUUGAAAAAG |
| | | GCAUUUCCUGAAAACAUCUCCAAGGAACGG |
| | | CCGCUCAUCAACAAGAACAUUCCCAAUUUC |
| | | AAGUGGCUGGCGGGGUUCACUGCCGGGGAC |
| | | GGACACUUCGGAGUGAACCUGAAGAAGGUG |
| | | AAGGGCACCGCCAAGGUGUACGUGGGCCUG |
| | | CGGUUCGCGAUCAGCCAGCACAUCCGGGAU |
| | | AAGAACCUGAUGAACAGCCUCAUCACCUAC |
| | | CUGGGAUGCGGAAGCAUCCGGGAGAAGAAC |
| | | AAGUCAGAAUUCCGAUGGCUGGAAUUUGAA |
| | | GUGACCAAGUUCUCCGACAUCAACGACAAG |
| | | AUCAUCCCCGUGUUCCAGGAGAACACCCUC |
| | | AUUGGAGUGAAGCUGGAGGACUUCGAGGAC |
| | | UGGUGCAAGGUGGCCAAGCUCAUCGAAGAG |
| | | AAGAAGCACCUGACCGAAAGCGGCCUGGAU |
| | | GAGAUUAAGAAGAUUAAGCUCAACAUGAAC |
| | | AAGGGAAGAUAGUAGCGCCGUACGGAAAAA |
| | | AAAAAAAAAAAAAAAAAAAAAAAAAAAAAA |
| | | AAAAAAAAAAAAAAAAAAAAAAAAAAAAAA |
| | | AAAAAAAAAAAAAAAAAAAAAAAAAAAAAA |
| | | AAAAAAAAAAAAAAAAAAAAAAAAAAAAAA |
| | | AAAAAAAAAAAAAAAAAAAAAAAAAAAAAA |
| | | AAAAAAAAAAAAAAAAAAAAAAAAAAAAAA |
| | | AAAAAAAAAAAAAAAAA |
| 3 | TCRα | CCUCCGAAGAAGAAGCGGAAAGUCGUGGAC |
| | megaTAL | CUCCGGACCCUGGGUUACUCUCAGCAGCAG |
| | RNA | CAGGAGAAGAUCAAGCCGAAGGUGCGGUCG |
| | | ACUGUGGCCCAGCAUCACGAGGCCCUGGUG |
| | | GGACACGGCUUCACCCACGCCCACAUUGUG |
| | | GCCCUGAGCCAGCACCCGGCAGCGCUGGGA |
| | | ACCGUGGCCGUGACCUACCAGCACAUCAUU |
| | | ACUGCCCUGCCUGAAGCGACCCACGAGGAU |
| | | AUCGUGGGCGUCGGAAAGCAGUGGUCCGGA |
| | | GCCAGAGCCUUGGAGGCUCUGCUGACUGAC |
| | | GCCGGAGAGCUGCGGGGCCCGCCCCUGCAA |
| | | CUGGAUACCGGCCAGCUCGUGAAAAUCGCC |
| | | AAGAGAGGAGGAGUGACCGCCAUGGAAGCC |
| | | GUGCAUGCAUCCCGCAAUGCACUGACUGGU |
| | | GCACCCCUGAACCUCACUCCUGACCAGGUC |
| | | GUCGCUAUCGCAAGCAACAUCGGAGGGAAA |
| | | CAAGCUCUCGAGACAGUGCAGCGCCUCCUG |
| | | CCAGUGCUUUGCCAGGACCACGGCCUGACU |
| | | CCAGACCAGGUGGUCGCUAUUGCGUCGAAC |
| | | AUUGGAGGGAAGCAAGCCCUUGAAACCGUG |
| | | CAGAGGCUGCUCCCGGUGCUGUGCCAAGAC |
| | | CAUGGACUCACCCCGGACCAAGUGGUGGCU |
| | | AUUGCUAGCAACAUUGGCGGUAAGCAGGCG |
| | | CUGGAGACAGUCCAGCGGCUGCUGCCGGUG |
| | | UUGUGCCAAGAUCACGGUCUUACCCCAGAC |
| | | CAAGUCGUGGCGAUUGCCUCCAACGGUGGC |
| | | GGCAAGCAAGCACUCGAAACUGUCCAGAGA |
| | | CUGCUCCCUGUGCUCUGUCAAGACCACGGG |
| | | UUGACCCCCGACCAAGUGGUGGCCAUCGCC |
| | | UCCCAUGAUGGAGGAAAGCAGGCCCUCGAG |
| | | ACUGUCCAGCGACUGCUCCCCGUGUUGUGU |
| | | CAGGAUCAUGGAUUGACGCCCGAUCAGGUC |
| | | GUGGCCAUUGCCUCCCACGACGGUGGAAAG |
| | | CAAGCGCUGGAAACUGUGCAGCGGUUGCUG |
| | | CCGGUCCUGUGCCAGGACCACGGACUGACU |
| | | CCGGACCAGGUGGUCGCCAUCGCAUCCAAC |
| | | AUUGGUGGCAAGCAGGCUCUCGAAACCGUC |
| | | CAACGCCUGUUGCCGGUGCUGUGUCAGGAU |
| | | CAUGGACUGACCCCGGACCAAGUGGUGGCU |
| | | AUCGCCUCCAACAACGGGGGCAAACAGGCC |
| | | CUGGAAACCGUGCAACGCCUGCUGCCCGUC |
| | | CUCUGCCAGGACCACGGUCUGACCCCUGAC |
| | | CAGGUCGUCGCGAUCGCGUCAAAUGGGGGG |
| | | GGCAAACAGGCUCUGGAAACGGUGCAGCGG |
| | | CUCCUUCCGGUGUUAUGCCAGGACCACGGG |
| | | CUGACUCCCGACCAGGUGGUGGCGAUCGCC |
| | | UCGAACAACGGAGGCAAACAAGCCCUGGAG |
| | | ACUGUGCAGAGACUCCUGCCCGUGCUGUGC |
| | | CAAGACCAUGGGCUCACCCCUGAUCAGGUG |
| | | GUGGCAAUCGCCUCAAACAUCGGCGGGAAG |
| | | CAGGCACUGGAAACUGUGCAGAGACUCCUG |
| | | CCCGUGCUGUGCCAAGACCAUGGGCUGACC |
| | | CCGGACCAAGUGGUGGCUAUCGCCUCCCAC |
| | | GACGGGGGCAAACAGGCCCUGGAAUCCAUC |
| | | GUCGCUCAGCUGAGCCGGCCUGACCCAGCA |
| | | CUCGCCGCCCUGACCAAUGACCAUCUGGUC |
| | | GCCCUGGCCUGCCUGGGAGGCAGACCCGCG |
| | | AUGGACGCGGUCAAGAAGGGUCUGCCGCAC |
| | | GCCCCUGAGCUUAUUCGGAGAGUGAACAGG |
| | | CGCAUCGGUGAACGCACCUCCCAUCGGGUC |
| | | GCAAUCUCUAGAGUGGGCGGAUCGUCCCGG |
| | | CGGGAGUCCAUCAAUCCUUGGAUCCUGACC |
| | | GGCUUCGCCGACGCCGAAGGCUCCUUCAUC |
| | | CUGGACAUCAGGAACAGGAACAACGAGUCA |
| | | AACAGGUACCGCACCUCCCUUCGGUUCCAG |
| | | AUUACUCUGCACAACAAGGAUAAGUCCAUC |
| | | CUCGAGAACAUCCAGUCAACCUGGAAAGUG |
| | | GGCAAGAUCACUAACUCCUCGGACCGCGCA |
| | | GUGAUGCUCCGGGUGACCCGCUUCGAGGAC |
| | | CUGAAGGUGAUCAUUGACCACUUCGAGAAG |
| | | UACCCUCUCAUAACCCAGAAGCUGGGAGAU |
| | | UACAAGCUGUUUAAGCAGGCGUUCUCCGUG |
| | | AUGGAGAACAAAGAACACCUUAAGGAGAAU |
| | | GGGAUUAAGGAACUGGUCCGCAUUAAGGCC |
| | | AAGAUGAACUGGGGACUGAACGACGAGUUG |
| | | AAAAAGGCAUUUCCUGAAAACAUCUCCAAG |
| | | GAACGGCCGCUCAUCAACAAGAACAUUCCC |
| | | AAUUUCAAGUGGCUGGCGGGGUUCACUGCC |
| | | GGGGACGGACACUUCGGAGUGAACCUGAAG |
| | | AAGGUGAAGGGCACCGCCAAGGUGUACGUG |
| | | GGCCUGCGGUUCGCGAUCAGCCAGCACAUC |
| | | CGGGAUAAGAACCUGAUGAACAGCCUCAUC |
| | | ACCUACCUGGGAUGCGGAAGCAUCCGGGAG |
| | | AAGAACAAGUCAGAAUUCCGAUGGCUGGAA |
| | | UUUGAAGUGACCAAGUUCUCCGACAUCAAC |
| | | GACAAGAUCAUCCCCGUGUUCCAGGAGAAC |
| | | ACCCUCAUUGGAGUGAAGCUGGAGGACUUC |
| | | GAGGACUGGUGCAAGGUGGCCAAGCUCAUC |
| | | GAAGAGAAGAAGCACCUGACCGAAAGCGGC |
| | | CUGGAUGAGAUUAAGAAGAUUAAGCUCAAC |
| | | AUGAACAAGGGAAGAUAGUAG |
| 4 | PD1 | CCGCCGAAGAAGAAGCGCAAGGTGGTGGAT |
| | megaTAL | CTGAGAACCCTGGGATACAGCCAGCAGCAG |
| | DNA | CAGGAGAAGATCAAGCCGAAGGTCCGGTCT |
| | | ACCGTGGCCCAGCACCATGAGGCCCTTGTG |
| | | GGCCACGGCTTCACACATGCACACATCGTC |
| | | GCCCTGTCGCAGCATCCCGCCGCCCTGGGG |
| | | ACCGTGGCCGTGACCTATCAACACATCATT |
| | | ACCGCCCTGCCGGAGGCCACCCACGAGGAC |
| | | ATCGTGGGTGTGGGGAAGCAGTGGAGCGGA |
| | | GCCAGGGCACTCGAAGCCCTCCTCACTGAC |
| | | GCTGGAGAACTGCGCGGACCGCCTCTCCAG |
| | | CTGGACACCGGACAGCTGGTGAAAATCGCC |
| | | AAGCGGGGAGGAGTGACCGCCATGGAAGCC |
| | | GTGCACGCCTCGAGGAACGCGCTGACTGGC |
| | | GCCCCTCTGAACCTGACCCCTGATCAGGTC |
| | | GTGGCTATCGCCTCAAACAACGGGGGTAAG |
| | | CAGGCGCTGGAGACAGTGCAACGACTTCTG |
| | | CCAGTGCTTTGTCAGGACCATGGTCTGACC |
| | | CCCGACCAGGTCGTCGCCATTGCATCCAAC |
| | | AATGGTGGCAAGCAGGCACTGGAGACTGTC |
| | | CAGAGGCTGCTCCCGGTGCTGTGCCAGGAC |
| | | CACGGGCTCACCCCGGACCAAGTGGTCGCC |
| | | ATCGCCTCCAACGGAGGAGGAAAACAAGCT |
| | | CTGGAGACTGTGCAACGCCTGCTGCCTGTG |
| | | TTGTGCCAAGACCACGGACTGACGCCCGAT |
| | | CAGGTGGTGGCGATCGCATCGAACAACGGA |
| | | GGAAAGCAAGCGCTGGAAACCGTGCAGCGC |
| | | CTCCTGCCCGTCCTCTGCCAGGATCACGGC |
| | | CTGACTCCGGACCAGGTGGTCGCGATCGCC |
| | | AGCAATAACGGGGGGAAGCAAGCCCTCGAG |
| | | ACTGTGCAGCGGTTGCTGCCCGTGCTCTGC |
| | | CAAGATCATGGCCTTACCCCAGACCAAGTC |
| | | GTGGCCATTGCTTCCAACAACGGTGGCAAA |
| | | CAGGCGCTCGAAACCGTCCAGCGGCTGTTG |
| | | CCCGTGCTTTGCCAGGATCACGGACTCACC |
| | | CCTGATCAGGTGGTGGCAATTGCGTCCAAC |
| | | AACGGTGGAAAGCAGGCCCTGGAAACGGTG |
| | | CAGCGGCTGCTTCCGGTCCTGTGTCAGGAT |
| | | CATGGGCTGACTCCCGACCAGGTCGTCGCC |
| | | ATTGCATCCCACGATGGGGGTAAACAGGCC |
| | | CTCGAAACAGTGCAGAGACTCCTGCCAGTC |
| | | CTGTGCCAAGACCACGGACTTACCCCGGAT |
| | | CAGGTGGTGGCCATAGCCTCGAACGGCGGC |
| | | GGGAAACAGGCTCTGGAAACTGTGCAAAGA |
| | | CTCCTCCCGGTGTTGTGTCAAGACCATGGA |
| | | CTGACCCCAGATCAGGTGGTGGCTATTGCC |
| | | TCTAACAACGGCGGCAAGCAAGCACTCGAA |
| | | AGCATCGTGGCCCAGTTGTCACGCCCCGAC |
| | | CCCGCACTGGCTGCCCTGACGAATGACCAT |
| | | CTGGTGGCGCTGGCCTGCCTGGGAGGGAGG |
| | | CCAGCGATGGATGCGGTGAAGAAGGGACTG |
| | | CCCCATGCTCCGGAGCTGATTCGGAGAGTG |
| | | AATAGGCGCATCGGAGAGAGAACTTCACAT |
| | | CGGGTGGCCATTTCTAGAGTGGGCGGCAGC |
| | | TCCCGGCGCGAGTCCATTAACCCCTGGATC |
| | | CTGACCGGCTTTGCCGACGCCGAAGGGTCC |
| | | TTCGGCCTCTCGATCCTGAACCGGAACCGG |
| | | GGTACCGCTCGGTACCACACCAGACTGTCC |
| | | TTCACCATCGTGCTGCACAACAAGGACAAG |
| | | AGCATCCTCGAAAACATTCAGTCAACGTGG |
| | | AAGGTGGGAATTATTACTAACGACGGCGAC |
| | | AGATACGTGCGCCTGTGCGTGACCCGGTTT |
| | | GAGGACCTGAAGGTCATTATCGACCACTTC |
| | | GAGAAGTACCCCCTCGTGACTCAGAAGCTG |
| | | GGAGACTACAAGCTGTTCAAGCAGGCGTTC |
| | | TCGGTGATGGAAAACAAGGAGCACCTGAAG |
| | | GAGAACGGCATCAAGGAGCTCGCCCGGATC |
| | | AAGGCCAAGATGAACTGGGGCCTGAATGAT |
| | | GAACTCAAGAAGGCGTTCCCTGAAAACATC |
| | | GGTAAAGAACGGCCCCTGATCAACAAGAAC |
| | | ATCCCGAACTTCAAGTGGCTTGCCGGATTC |
| | | ACCTCCGGCGACGGATCCTTCTTCGTCCGG |
| | | CTGCGCAAGTCCAACGTGAACGCGAGAGTG |
| | | CGGGTGCAATTGGTCTTTGAAATCTCACAG |
| | | CACATCAGGGACAAGAATTTGATGAACTCC |
| | | CTCATCACCTACCTGGGTTGCGGACACATC |
| | | TACGAAGGCAATAAGTCGGAGCGGAGCTGG |
| | | CTGCAGTTTCGCGTGGAAAAGTTCTCCGAC |
| | | ATTAACGACAAGATCATCCCAGTGTTCCAG |
| | | GAAAACACTCTGATTGGCGTGAAGCTTGAG |
| | | GATTTCGAGGACTGGTGCAAGGTGGCCAAG |
| | | CTGATTGAAGAGAAGAAGCACCTGACCGAG |
| | | TCCGGCCTGGACGAAATCAAGAAAATCAAG |
| | | CTGAACATGAACAAGGGACGG |
| 5 | PD1 megaTAL | GGGGCCGCCACCAUGGGAAGCUGCCGGUAC |
| | RNA | CCCUACGACGUCCCUGACUACGCCCCGCCG |
| | | AAGAAGAAGCGCAAGGUGGUGGAUCUGAGA |
| | | ACCCUGGGAUACAGCCAGCAGCAGCAGGAG |
| | | AAGAUCAAGCCGAAGGUCCGGUCUACCGUG |
| | | GCCCAGCACCAUGAGGCCCUUGUGGGCCAC |
| | | GGCUUCACACAUGCACACAUCGUCGCCCUG |
| | | UCGCAGCAUCCCGCCGCCCUGGGGACCGUG |
| | | GCCGUGACCUAUCAACACAUCAUUACCGCC |
| | | CUGCCGGAGGCCACCCACGAGGACAUCGUG |
| | | GGUGUGGGGAAGCAGUGGAGCGGAGCCAGG |
| | | GCACUCGAAGCCCUCCUCACUGACGCUGGA |
| | | GAACUGCGCGGACCGCCUCUCCAGCUGGAC |
| | | ACCGGACAGCUGGUGAAAAUCGCCAAGCGG |
| | | GGAGGAGUGACCGCCAUGGAAGCCGUGCAC |
| | | GCCUCGAGGAACGCGCUGACUGGCGCCCCU |
| | | CUGAACCUGACCCCUGAUCAGGUCGUGGCU |
| | | AUCGCCUCAAACAACGGGGGUAAGCAGGCG |
| | | CUGGAGACAGUGCAACGACUUCUGCCAGUG |
| | | CUUUGUCAGGACCAUGGUCUGACCCCCGAC |
| | | CAGGUCGUCGCCAUUGCAUCCAACAAUGGU |
| | | GGCAAGCAGGCACUGGAGACUGUCCAGAGG |
| | | CUGCUCCCGGUGCUGUGCCAGGACCACGGG |
| | | CUCACCCCGGACCAAGUGGUCGCCAUCGCC |
| | | UCCAACGGAGGAGGAAAACAAGCUCUGGAG |
| | | ACUGUGCAACGCCUGCUGCCUGUGUUGUGC |
| | | CAAGACCACGGACUGACGCCCGAUCAGGUG |
| | | GUGGCGAUCGCAUCGAACAACGGAGGAAAG |
| | | CAAGCGCUGGAAACCGUGCAGCGCCUCCUG |
| | | CCCGUCCUCUGCCAGGAUCACGGCCUGACU |
| | | CCGGACCAGGUGGUCGCGAUCGCCAGCAAU |
| | | AACGGGGGGAAGCAAGCCCUCGAGACUGUG |
| | | CAGCGGUUGCUGCCCGUGCUCUGCCAAGAU |
| | | CAUGGCCUUACCCCAGACCAAGUCGUGGCC |
| | | AUUGCUUCCAACAACGGUGGCAAACAGGCG |
| | | CUCGAAACCGUCCAGCGGCUGUUGCCCGUG |
| | | CUUUGCCAGGAUCACGGACUCACCCCUGAU |
| | | CAGGUGGUGGCAAUUGCGUCCAACAACGGU |
| | | GGAAAGCAGGCCCUGGAAACGGUGCAGCGG |
| | | CUGCUUCCGGUCCUGUGUCAGGAUCAUGGG |
| | | CUGACUCCCGACCAGGUCGUCGCCAUUGCA |
| | | UCCCACGAUGGGGGUAAACAGGCCCUCGAA |
| | | ACAGUGCAGAGACUCCUGCCAGUCCUGUGC |
| | | CAAGACCACGGACUUACCCCGGAUCAGGUG |
| | | GUGGCCAUAGCCUCGAACGGCGGCGGGAAA |
| | | CAGGCUCUGGAAACUGUGCAAAGACUCCUC |
| | | CCGGUGUUGUGUCAAGACCAUGGACUGACC |
| | | CCAGAUCAGGUGGUGGCUAUUGCCUCUAAC |
| | | AACGGCGGCAAGCAAGCACUCGAAAGCAUC |
| | | GUGGCCCAGUUGUCACGCCCCGACCCCGCA |
| | | CUGGCUGCCCUGACGAAUGACCAUCUGGUG |
| | | GCGCUGGCCUGCCUGGGAGGGAGGCCAGCG |
| | | AUGGAUGCGGUGAAGAAGGGACUGCCCCAU |
| | | GCUCCGGAGCUGAUUCGGAGAGUGAAUAGG |
| | | CGCAUCGGAGAGAGAACUUCACAUCGGGUG |
| | | GCCAUUUCUAGAGUGGGCGGCAGCUCCCGG |
| | | CGCGAGUCCAUUAACCCCUGGAUCCUGACC |
| | | GGCUUUGCCGACGCCGAAGGGUCCUUCGGC |
| | | CUCUCGAUCCUGAACCGGAACCGGGGUACC |
| | | GCUCGGUACCACACCAGACUGUCCUUCACC |
| | | AUCGUGCUGCACAACAAGGACAAGAGCAUC |
| | | CUCGAAAACAUUCAGUCAACGUGGAAGGUG |
| | | GGAAUUAUUACUAACGACGGCGACAGAUAC |
| | | GUGCGCCUGUGCGUGACCCGGUUUGAGGAC |
| | | CUGAAGGUCAUUAUCGACCACUUCGAGAAG |
| | | UACCCCCUCGUGACUCAGAAGCUGGGAGAC |
| | | UACAAGCUGUUCAAGCAGGCGUUCUCGGUG |
| | | AUGGAAAACAAGGAGCACCUGAAGGAGAAC |
| | | GGCAUCAAGGAGCUCGCCCGGAUCAAGGCC |
| | | AAGAUGAACUGGGGCCUGAAUGAUGAACUC |
| | | AAGAAGGCGUUCCCUGAAAACAUCGGUAAA |
| | | GAACGGCCCCUGAUCAACAAGAACAUCCCG |
| | | AACUUCAAGUGGCUUGCCGGAUUCACCUCC |
| | | GGCGACGGAUCCUUCUUCGUCCGGCUGCGC |
| | | AAGUCCAACGUGAACGCGAGAGUGCGGGUG |
| | | CAAUUGGUCUUUGAAAUCUCACAGCACAUC |
| | | AGGGACAAGAAUUUGAUGAACUCCCUCAUC |
| | | ACCUACCUGGGUUGCGGACACAUCUACGAA |
| | | GGCAAUAAGUCGGAGCGGAGCUGGCUGCAG |
| | | UUUCGCGUGGAAAAGUUCUCCGACAUUAAC |
| | | GACAAGAUCAUCCCAGUGUUCCAGGAAAAC |
| | | ACUCUGAUUGGCGUGAAGCUUGAGGAUUUC |
| | | GAGGACUGGUGCAAGGUGGCCAAGCUGAUU |
| | | GAAGAGAAGAAGCACCUGACCGAGUCCGGC |
| | | CUGGACGAAAUCAAGAAAAUCAAGCUGAAC |
| | | AUGAACAAGGGACGGUGAUAGCGCGCUAGC |
| | | CGUACGGAAAAAAAAAAAAAAAAAAAAAAA |
| | | AAAAAAAAAAAAAAAAAAAAAAAAAAAAAA |
| | | AAAAAAAAAAAAAAAAAAAAAAAAAAAAAA |
| | | AAAAAAAAAAAAAAAAAAAAAAAAAAAAAA |
| | | AAAAAAAAAAAAAAAAAAAAAAAAAAAAAA |
| | | AAAAAAAAAAAAAAAAAAAAAAAAAAAAAA |
| | | AAAAAAAAAAAAAAAAAAAAAAAAAAAAA |
| 6 | PD1 megaTAL | CCGCCGAAGAAGAAGCGCAAGGUGGUGGAU |
| | RNA | CUGAGAACCCUGGGAUACAGCCAGCAGCAG |
| | | CAGGAGAAGAUCAAGCCGAAGGUCCGGUCU |
| | | ACCGUGGCCCAGCACCAUGAGGCCCUUGUG |
| | | GGCCACGGCUUCACACAUGCACACAUCGUC |
| | | GCCCUGUCGCAGCAUCCCGCCGCCCUGGGG |
| | | ACCGUGGCCGUGACCUAUCAACACAUCAUU |
| | | ACCGCCCUGCCGGAGGCCACCCACGAGGAC |
| | | AUCGUGGGUGUGGGGAAGCAGUGGAGCGGA |
| | | GCCAGGGCACUCGAAGCCCUCCUCACUGAC |
| | | GCUGGAGAACUGCGCGGACCGCCUCUCCAG |
| | | CUGGACACCGGACAGCUGGUGAAAAUCGCC |
| | | AAGCGGGGAGGAGUGACCGCCAUGGAAGCC |
| | | GUGCACGCCUCGAGGAACGCGCUGACUGGC |
| | | GCCCCUCUGAACCUGACCCCUGAUCAGGUC |
| | | GUGGCUAUCGCCUCAAACAACGGGGGUAAG |
| | | CAGGCGCUGGAGACAGUGCAACGACUUCUG |
| | | CCAGUGCUUUGUCAGGACCAUGGUCUGACC |
| | | CCCGACCAGGUCGUCGCCAUUGCAUCCAAC |
| | | AAUGGUGGCAAGCAGGCACUGGAGACUGUC |
| | | CAGAGGCUGCUCCCGGUGCUGUGCCAGGAC |
| | | CACGGGCUCACCCCGGACCAAGUGGUCGCC |
| | | AUCGCCUCCAACGGAGGAGGAAAACAAGCU |
| | | CUGGAGACUGUGCAACGCCUGCUGCCUGUG |
| | | UUGUGCCAAGACCACGGACUGACGCCCGAU |
| | | CAGGUGGUGGCGAUCGCAUCGAACAACGGA |
| | | GGAAAGCAAGCGCUGGAAACCGUGCAGCGC |
| | | CUCCUGCCCGUCCUCUGCCAGGAUCACGGC |
| | | CUGACUCCGGACCAGGUGGUCGCGAUCGCC |
| | | AGCAAUAACGGGGGGAAGCAAGCCCUCGAG |
| | | ACUGUGCAGCGGUUGCUGCCCGUGCUCUGC |
| | | CAAGAUCAUGGCCUUACCCCAGACCAAGUC |
| | | GUGGCCAUUGCUUCCAACAACGGUGGCAAA |
| | | CAGGCGCUCGAAACCGUCCAGCGGCUGUUG |
| | | CCCGUGCUUUGCCAGGAUCACGGACUCACC |
| | | CCUGAUCAGGUGGUGGCAAUUGCGUCCAAC |
| | | AACGGUGGAAAGCAGGCCCUGGAAACGGUG |
| | | CAGCGGCUGCUUCCGGUCCUGUGUCAGGAU |
| | | CAUGGGCUGACUCCCGACCAGGUCGUCGCC |
| | | AUUGCAUCCCACGAUGGGGGUAAACAGGCC |
| | | CUCGAAACAGUGCAGAGACUCCUGCCAGUC |
| | | CUGUGCCAAGACCACGGACUUACCCCGGAU |
| | | CAGGUGGUGGCCAUAGCCUCGAACGGCGGC |
| | | GGGAAACAGGCUCUGGAAACUGUGCAAAGA |
| | | CUCCUCCCGGUGUUGUGUCAAGACCAUGGA |
| | | CUGACCCCAGAUCAGGUGGUGGCUAUUGCC |
| | | UCUAACAACGGCGGCAAGCAAGCACUCGAA |
| | | AGCAUCGUGGCCCAGUUGUCACGCCCCGAC |
| | | CCCGCACUGGCUGCCCUGACGAAUGACCAU |
| | | CUGGUGGCGCUGGCCUGCCUGGGAGGGAGG |
| | | CCAGCGAUGGAUGCGGUGAAGAAGGGACUG |
| | | CCCCAUGCUCCGGAGCUGAUUCGGAGAGUG |
| | | AAUAGGCGCAUCGGAGAGAGAACUUCACAU |
| | | CGGGUGGCCAUUUCUAGAGUGGGCGGCAGC |
| | | UCCCGGCGCGAGUCCAUUAACCCCUGGAUC |
| | | CUGACCGGCUUUGCCGACGCCGAAGGGUCC |
| | | UUCGGCCUCUCGAUCCUGAACCGGAACCGG |
| | | GGUACCGCUCGGUACCACACCAGACUGUCC |
| | | UUCACCAUCGUGCUGCACAACAAGGACAAG |
| | | AGCAUCCUCGAAAACAUUCAGUCAACGUGG |
| | | AAGGUGGGAAUUAUUACUAACGACGGCGAC |
| | | AGAUACGUGCGCCUGUGCGUGACCCGGUUU |
| | | GAGGACCUGAAGGUCAUUAUCGACCACUUC |
| | | GAGAAGUACCCCCUCGUGACUCAGAAGCUG |
| | | GGAGACUACAAGCUGUUCAAGCAGGCGUUC |
| | | UCGGUGAUGGAAAACAAGGAGCACCUGAAG |
| | | GAGAACGGCAUCAAGGAGCUCGCCCGGAUC |
| | | AAGGCCAAGAUGAACUGGGGCCUGAAUGAU |
| | | GAACUCAAGAAGGCGUUCCCUGAAAACAUC |
| | | GGUAAAGAACGGCCCCUGAUCAACAAGAAC |
| | | AUCCCGAACUUCAAGUGGCUUGCCGGAUUC |
| | | ACCUCCGGCGACGGAUCCUUCUUCGUCCGG |
| | | CUGCGCAAGUCCAACGUGAACGCGAGAGUG |
| | | CGGGUGCAAUUGGUCUUUGAAAUCUCACAG |
| | | CACAUCAGGGACAAGAAUUUGAUGAACUCC |
| | | CUCAUCACCUACCUGGGUUGCGGACACAUC |
| | | UACGAAGGCAAUAAGUCGGAGCGGAGCUGG |
| | | CUGCAGUUUCGCGUGGAAAAGUUCUCCGAC |
| | | AUUAACGACAAGAUCAUCCCAGUGUUCCAG |
| | | GAAAACACUCUGAUUGGCGUGAAGCUUGAG |
| | | GAUUUCGAGGACUGGUGCAAGGUGGCCAAG |
| | | CUGAUUGAAGAGAAGAAGCACCUGACCGAG |
| | | UCCGGCCUGGACGAAAUCAAGAAAAUCAAG |
| | | CUGAACAUGAACAAGGGACGG |
| 7 | PCSK9 | CCTCCTAAGAAGAAGCGCAAGGTCGTGGAT |
| | megaTAL | CTGCGGACCCTAGGTTACTCCCAGCAGCAG |
| | DNA | CAGGAAAAGATTAAGCCAAAGGTCCGCTCC |
| | | ACCGTGGCGCAGCACCACGAAGCACTTGTG |
| | | GGGCACGGATTCACCCACGCCCACATCGTG |
| | | GCCCTGTCCCAACATCCGGCCGCCCTGGGT |
| | | ACTGTCGCCGTGACTTATCAGCACATTATC |
| | | ACTGCCCTGCCCGAGGCCACCCACGAAGAT |
| | | ATTGTGGGAGTCGGAAAGCAGTGGTCAGGA |
| | | GCCCGCGCTCTCGAGGCCCTCCTGACCGAT |
| | | GCTGGAGAGCTGAGGGGTCCTCCGCTCCAG |
| | | CTCGACACTGGACAGCTCGTGAAAATCGCT |
| | | AAGAGGGGTGGTGTGACCGCCATGGAAGCT |
| | | GTGCACGCCAGCCGGAACGCTCTGACTGGA |
| | | GCCCCCCTGAACCTGACTCCTGATCAGGTG |
| | | GTTGCTATAGCTTCGAACAATGGCGGTAAG |
| | | CAAGCATTGGAAACCGTCCAACGGCTGTTG |
| | | CCCGTGCTTTGTCAAGATCACGGACTGACC |
| | | CCCGACCAGGTTGTGGCCATTGCGTCCAAC |
| | | AATGGAGGCAAGCAAGCCCTGGAGACAGTG |
| | | CAGCGGTTGCTGCCGGTGTTGTGCCAAGAT |
| | | CATGGGTTAACGCCTGACCAGGTCGTCGCA |
| | | ATCGCCTCAAATAACGGGGGAAAGCAGGCC |
| | | CTCGAGACTGTGCAACGCCTCCTGCCGGTC |
| | | CTCTGCCAAGATCATGGCCTGACGCCGGAT |
| | | CAGGTCGTGGCTATCGCAAGCCACGACGGC |
| | | GGGAAGCAAGCACTCGAGACTGTGCAGAGA |
| | | CTGCTCCCGGTCCTGTGTCAAGACCATGGA |
| | | CTCACGCCCGATCAAGTCGTCGCGATTGCC |
| | | AGCAACATTGGAGGGAAACAAGCTCTCGAA |
| | | ACCGTGCAACGCTTGCTGCCCGTCCTGTGC |
| | | CAAGACCACGGACTCACCCCCGATCAGGTG |
| | | GTGGCAATCGCCAGCCACGATGGGGGGAAA |
| | | CAGGCATTGGAAACTGTGCAAAGACTGCTG |
| | | CCAGTCTTGTGCCAGGACCACGGGCTCACC |
| | | CCTGATCAAGTGGTGGCGATCGCAAGCCAT |
| | | GACGGAGGAAAACAAGCCTTGGAGACTGTC |
| | | CAGAGGCTCCTTCCCGTGTTGTGTCAAGAC |
| | | CATGGCCTCACTCCGGACCAAGTCGTGGCC |
| | | ATCGCCTCGCACGACGGAGGCAAACAAGCA |
| | | CTGGAAACCGTCCAGAGATTGCTGCCTGTC |
| | | CTGTGCCAAGACCACGGTCTGACTCCCGAC |
| | | CAAGTGGTCGCCATTGCATCCAACGGAGGT |
| | | GGCAAGCAGGCTTTGGAAACCGTACAACGG |
| | | CTGCTGCCTGTGCTCTGCCAGGACCACGGG |
| | | CTCACCCCGGATCAAGTIGTGGCTATTGCC |
| | | TCCAATAACGGCGGAAAACAGGCCCTGGAA |
| | | ACGGTCCAGCGGCTGCTTCCGGTGCTGTGC |
| | | CAGGATCACGGGCTGACACCGGACCAGGTG |
| | | GTCGCTATCGCCTCCAACAACGGGGGGAAG |
| | | CAGGCTCTCGAAACCGTGCAGAGGCTGCTC |
| | | CCTGTGCTTTGCCAGGATCACGGCCTTACC |
| | | CCTGACCAGGTCGTCGCCATCGCTTCGAAC |
| | | ATCGGTGGTAAGCAGGCGCTGGAAACTGTC |
| | | CAACGCCTTCTTCCAGTGCTGTGTCAGGAC |
| | | CATGGGCTTACCCCAGACCAGGTAGTCGCT |
| | | ATAGCATCAAACGGTGGAGGAAAGCAGGCC |
| | | TTGGAAACGGTGCAGCGGCTCCTGCCAGTC |
| | | CTGTGCCAAGACCACGGCCTGACCCCAGAT |
| | | CAGGTGGTAGCCATCGCCAGCAATAACGGT |
| | | GGCAAACAGGCTCTGGAGTCCATTGTGGCC |
| | | CAGCTGTCGAGGCCTGACCCGGCGCTGGCC |
| | | GCCCTGACCAACGATCACCTGGTCGCGCTG |
| | | GCCTGTCTCGGAGGCAGACCCGCTATGGAC |
| | | GCAGTGAAGAAAGGCCTGCCCCATGCCCCG |
| | | GAACTGATCCGCAGGGTGAATCGCCGGATC |
| | | GGCGAAAGAACCAGCCATCGAGTGGCCATC |
| | | TCCCGCGTGGGCGGCTCCTCCAGACGCGAG |
| | | TCCATAAACCCGTGGATCCTGACCGGGTTC |
| | | GCCGATGCCGAAGGTTGCTTCATGCTGAAT |
| | | ATTTGGAATAAGAACCGAACTCGGGCCAAA |
| | | TACTACACCGCTCTCCGCTTCGAGATCGCG |
| | | CTCCACAACAAGGATAAGTCCATACTGGAG |
| | | AACATCCGATCGACCTGGAAAGTCGGCAAG |
| | | ATCCGCAACATCGGGGACCGGGTGGTGAAG |
| | | CTGGTCGTGGGACGGTTCGAAGATTTGAAG |
| | | GTCATTATCGACCATTTCGAGAAGTACCCT |
| | | CTGATTACGCAGAAGCTGGGGGACTACAAG |
| | | CTGTTCAAACAGGCTTTCTCCCTGATGGAG |
| | | AACAAGGAACACTTGAAGGAAAACGGCATC |
| | | AAGGAACTGGTCCGCATCAAGGCCAAGATG |
| | | AACTGGGGGCTGAACGACGAGCTGAAGAAG |
| | | GCCTTCCCGGAAAACATTAGCAAGGAGCGC |
| | | CCGCTCATCAACAAGAACATCCCCAACCTG |
| | | AAGTGGCTGGCGGGATTCACTTCCGGCGAC |
| | | GGTTATTTTGGAGTGGAACTCATGAAGAGA |
| | | ACAACCGGAACCCACGTGACCGTGCGACTC |
| | | CGGTTCTCCATTACCCAGCATATCCGGGAC |
| | | AAAAACCTGATGAACAGCCTGATTACATAC |
| | | TTGGGATGTGGCCGCATTTCCGAGCGGAAC |
| | | AAGTCCGAATACAGCTGGCTGGAATTCGTG |
| | | GTCACCAAGTTCAGCGACATCAACGACAAG |
| | | ATCATCCCAGTGTTCCAGGAGAATACCCTG |
| | | ATTGGAGTGAAGCTCGAGGATTTTGAGGAC |
| | | TGGTGCAAGGTCGCCAAGCTGATAGAAGAA |
| | | AAGAAGCATCTCACCGAATCAGGCCTGGAT |
| | | GAAATTAAGAAGATCAAACTCAACATGAAT |
| | | AAGGGACGCGTGTTC |
| 8 | PCSK9 | GGGGCCGCCACCAUGGGGUCAUGUCGGUAC |
| | megaTAL | CCCUACGACGUCCCCGACUACGCGCCUCCU |
| | RNA | AAGAAGAAGCGCAAGGUCGUGGAUCUGCGG |
| | | ACCCUAGGUUACUCCCAGCAGCAGCAGGAA |
| | | AAGAUUAAGCCAAAGGUCCGCUCCACCGUG |
| | | GCGCAGCACCACGAAGCACUUGUGGGGCAC |
| | | GGAUUCACCCACGCCCACAUCGUGGCCCUG |
| | | UCCCAACAUCCGGCCGCCCUGGGUACUGUC |
| | | GCCGUGACUUAUCAGCACAUUAUCACUGCC |
| | | CUGCCCGAGGCCACCCACGAAGAUAUUGUG |
| | | GGAGUCGGAAAGCAGUGGUCAGGAGCCCGC |
| | | GCUCUCGAGGCCCUCCUGACCGAUGCUGGA |
| | | GAGCUGAGGGGUCCUCCGCUCCAGCUCGAC |
| | | ACUGGACAGCUCGUGAAAAUCGCUAAGAGG |
| | | GGUGGUGUGACCGCCAUGGAAGCUGUGCAC |
| | | GCCAGCCGGAACGCUCUGACUGGAGCCCCC |
| | | CUGAACCUGACUCCUGAUCAGGUGGUUGCU |
| | | AUAGCUUCGAACAAUGGCGGUAAGCAAGCA |
| | | UUGGAAACCGUCCAACGGCUGUUGCCCGUG |
| | | CUUUGUCAAGAUCACGGACUGACCCCCGAC |
| | | CAGGUUGUGGCCAUUGCGUCCAACAAUGGA |
| | | GGCAAGCAAGCCCUGGAGACAGUGCAGCGG |
| | | UUGCUGCCGGUGUUGUGCCAAGAUCAUGGG |
| | | UUAACGCCUGACCAGGUCGUCGCAAUCGCC |
| | | UCAAAUAACGGGGGAAAGCAGGCCCUCGAG |
| | | ACUGUGCAACGCCUCCUGCCGGUCCUCUGC |
| | | CAAGAUCAUGGCCUGACGCCGGAUCAGGUC |
| | | GUGGCUAUCGCAAGCCACGACGGCGGGAAG |
| | | CAAGCACUCGAGACUGUGCAGAGACUGCUC |
| | | CCGGUCCUGUGUCAAGACCAUGGACUCACG |
| | | CCCGAUCAAGUCGUCGCGAUUGCCAGCAAC |
| | | AUUGGAGGGAAACAAGCUCUCGAAACCGUG |
| | | CAACGCUUGCUGCCCGUCCUGUGCCAAGAC |
| | | CACGGACUCACCCCCGAUCAGGUGGUGGCA |
| | | AUCGCCAGCCACGAUGGGGGGAAACAGGCA |
| | | UUGGAAACUGUGCAAAGACUGCUGCCAGUC |
| | | UUGUGCCAGGACCACGGGCUCACCCCUGAU |
| | | CAAGUGGUGGCGAUCGCAAGCCAUGACGGA |
| | | GGAAAACAAGCCUUGGAGACUGUCCAGAGG |
| | | CUCCUUCCCGUGUUGUGUCAAGACCAUGGC |
| | | CUCACUCCGGACCAAGUCGUGGCCAUCGCC |
| | | UCGCACGACGGAGGCAAACAAGCACUGGAA |
| | | ACCGUCCAGAGAUUGCUGCCUGUCCUGUGC |
| | | CAAGACCACGGUCUGACUCCCGACCAAGUG |
| | | GUCGCCAUUGCAUCCAACGGAGGUGGCAAG |
| | | CAGGCUUUGGAAACCGUACAACGGCUGCUG |
| | | CCUGUGCUCUGCCAGGACCACGGGCUCACC |
| | | CCGGAUCAAGUUGUGGCUAUUGCCUCCAAU |
| | | AACGGCGGAAAACAGGCCCUGGAAACGGUC |
| | | CAGCGGCUGCUUCCGGUGCUGUGCCAGGAU |
| | | CACGGGCUGACACCGGACCAGGUGGUCGCU |
| | | AUCGCCUCCAACAACGGGGGGAAGCAGGCU |
| | | CUCGAAACCGUGCAGAGGCUGCUCCCUGUG |
| | | CUUUGCCAGGAUCACGGCCUUACCCCUGAC |
| | | CAGGUCGUCGCCAUCGCUUCGAACAUCGGU |
| | | GGUAAGCAGGCGCUGGAAACUGUCCAACGC |
| | | CUUCUUCCAGUGCUGUGUCAGGACCAUGGG |
| | | CUUACCCCAGACCAGGUAGUCGCUAUAGCA |
| | | UCAAACGGUGGAGGAAAGCAGGCCUUGGAA |
| | | ACGGUGCAGCGGCUCCUGCCAGUCCUGUGC |
| | | CAAGACCACGGCCUGACCCCAGAUCAGGUG |
| | | GUAGCCAUCGCCAGCAAUAACGGUGGCAAA |
| | | CAGGCUCUGGAGUCCAUUGUGGCCCAGCUG |
| | | UCGAGGCCUGACCCGGCGCUGGCCGCCCUG |
| | | ACCAACGAUCACCUGGUCGCGCUGGCCUGU |
| | | CUCGGAGGCAGACCCGCUAUGGACGCAGUG |
| | | AAGAAAGGCCUGCCCCAUGCCCCGGAACUG |
| | | AUCCGCAGGGUGAAUCGCCGGAUCGGCGAA |
| | | AGAACCAGCCAUCGAGUGGCCAUCUCCCGC |
| | | GUGGGCGGCUCCUCCAGACGCGAGUCCAUA |
| | | AACCCGUGGAUCCUGACCGGGUUCGCCGAU |
| | | GCCGAAGGUUGCUUCAUGCUGAAUAUUUGG |
| | | AAUAAGAACCGAACUCGGGCCAAAUACUAC |
| | | ACCGCUCUCCGCUUCGAGAUCGCGCUCCAC |
| | | AACAAGGAUAAGUCCAUACUGGAGAACAUC |
| | | CGAUCGACCUGGAAAGUCGGCAAGAUCCGC |
| | | AACAUCGGGGACCGGGUGGUGAAGCUGGUC |
| | | GUGGGACGGUUCGAAGAUUUGAAGGUCAUU |
| | | AUCGACCAUUUCGAGAAGUACCCUCUGAUU |
| | | ACGCAGAAGCUGGGGGACUACAAGCUGUUC |
| | | AAACAGGCUUUCUCCCUGAUGGAGAACAAG |
| | | GAACACUUGAAGGAAAACGGCAUCAAGGAA |
| | | CUGGUCCGCAUCAAGGCCAAGAUGAACUGG |
| | | GGGCUGAACGACGAGCUGAAGAAGGCCUUC |
| | | CCGGAAAACAUUAGCAAGGAGCGCCCGCUC |
| | | AUCAACAAGAACAUCCCCAACCUGAAGUGG |
| | | CUGGCGGGAUUCACUUCCGGCGACGGUUAU |
| | | UUUGGAGUGGAACUCAUGAAGAGAACAACC |
| | | GGAACCCACGUGACCGUGCGACUCCGGUUC |
| | | UCCAUUACCCAGCAUAUCCGGGACAAAAAC |
| | | CUGAUGAACAGCCUGAUUACAUACUUGGGA |
| | | UGUGGCCGCAUUUCCGAGCGGAACAAGUCC |
| | | GAAUACAGCUGGCUGGAAUUCGUGGUCACC |
| | | AAGUUCAGCGACAUCAACGACAAGAUCAUC |
| | | CCAGUGUUCCAGGAGAAUACCCUGAUUGGA |
| | | GUGAAGCUCGAGGAUUUUGAGGACUGGUGC |
| | | AAGGUCGCCAAGCUGAUAGAAGAAAAGAAG |
| | | CAUCUCACCGAAUCAGGCCUGGAUGAAAUU |
| | | AAGAAGAUCAAACUCAACAUGAAUAAGGGA |
| | | CGCGUGUUCAGCGGACGCUAGUGACGCGCU |
| | | AGCCAUUUAAAUGUUAAUCGUACGGAAAAA |
| | | AAAAAAAAAAAAAAAAAAAAAAAAAAAAAA |
| | | AAAAAAAAAAAAAAAAAAAAAAAAAAAAAA |
| | | AAAAAAAAAAAAAAAAAAAAAAAAAAAAAA |
| | | AAAAAAAAAAAAAAAAAAAAAAAAAAAAAA |
| | | AAAAAAAAAAAAAAAAAAAAAAAAAAAAAA |
| | | AAAAAAAAAAAAAAAAAAAAAAAAAAAAAA |
| | | AAAAAAAAAAAAAAAAA |
| 9 | PCSK9 | CCUCCUAAGAAGAAGCGCAAGGUCGUGGAU |
| | megaTAL | CUGCGGACCCUAGGUUACUCCCAGCAGCAG |
| | RNA | CAGGAAAAGAUUAAGCCAAAGGUCCGCUCC |
| | | ACCGUGGCGCAGCACCACGAAGCACUUGUG |
| | | GGGCACGGAUUCACCCACGCCCACAUCGUG |
| | | GCCCUGUCCCAACAUCCGGCCGCCCUGGGU |
| | | ACUGUCGCCGUGACUUAUCAGCACAUUAUC |
| | | ACUGCCCUGCCCGAGGCCACCCACGAAGAU |
| | | AUUGUGGGAGUCGGAAAGCAGUGGUCAGGA |
| | | GCCCGCGCUCUCGAGGCCCUCCUGACCGAU |
| | | GCUGGAGAGCUGAGGGGUCCUCCGCUCCAG |
| | | CUCGACACUGGACAGCUCGUGAAAAUCGCU |
| | | AAGAGGGGUGGUGUGACCGCCAUGGAAGCU |
| | | GUGCACGCCAGCCGGAACGCUCUGACUGGA |
| | | GCCCCCCUGAACCUGACUCCUGAUCAGGUG |
| | | GUUGCUAUAGCUUCGAACAAUGGCGGUAAG |
| | | CAAGCAUUGGAAACCGUCCAACGGCUGUUG |
| | | CCCGUGCUUUGUCAAGAUCACGGACUGACC |
| | | CCCGACCAGGUUGUGGCCAUUGCGUCCAAC |
| | | AAUGGAGGCAAGCAAGCCCUGGAGACAGUG |
| | | CAGCGGUUGCUGCCGGUGUUGUGCCAAGAU |
| | | CAUGGGUUAACGCCUGACCAGGUCGUCGCA |
| | | AUCGCCUCAAAUAACGGGGGAAAGCAGGCC |
| | | CUCGAGACUGUGCAACGCCUCCUGCCGGUC |
| | | CUCUGCCAAGAUCAUGGCCUGACGCCGGAU |
| | | CAGGUCGUGGCUAUCGCAAGCCACGACGGC |
| | | GGGAAGCAAGCACUCGAGACUGUGCAGAGA |
| | | CUGCUCCCGGUCCUGUGUCAAGACCAUGGA |
| | | CUCACGCCCGAUCAAGUCGUCGCGAUUGCC |
| | | AGCAACAUUGGAGGGAAACAAGCUCUCGAA |
| | | ACCGUGCAACGCUUGCUGCCCGUCCUGUGC |
| | | CAAGACCACGGACUCACCCCCGAUCAGGUG |
| | | GUGGCAAUCGCCAGCCACGAUGGGGGGAAA |
| | | CAGGCAUUGGAAACUGUGCAAAGACUGCUG |
| | | CCAGUCUUGUGCCAGGACCACGGGCUCACC |
| | | CCUGAUCAAGUGGUGGCGAUCGCAAGCCAU |
| | | GACGGAGGAAAACAAGCCUUGGAGACUGUC |
| | | CAGAGGCUCCUUCCCGUGUUGUGUCAAGAC |
| | | CAUGGCCUCACUCCGGACCAAGUCGUGGCC |
| | | AUCGCCUCGCACGACGGAGGCAAACAAGCA |
| | | CUGGAAACCGUCCAGAGAUUGCUGCCUGUC |
| | | CUGUGCCAAGACCACGGUCUGACUCCCGAC |
| | | CAAGUGGUCGCCAUUGCAUCCAACGGAGGU |
| | | GGCAAGCAGGCUUUGGAAACCGUACAACGG |
| | | CUGCUGCCUGUGCUCUGCCAGGACCACGGG |
| | | CUCACCCCGGAUCAAGUUGUGGCUAUUGCC |
| | | UCCAAUAACGGCGGAAAACAGGCCCUGGAA |
| | | ACGGUCCAGCGGCUGCUUCCGGUGCUGUGC |
| | | CAGGAUCACGGGCUGACACCGGACCAGGUG |
| | | GUCGCUAUCGCCUCCAACAACGGGGGGAAG |
| | | CAGGCUCUCGAAACCGUGCAGAGGCUGCUC |
| | | CCUGUGCUUUGCCAGGAUCACGGCCUUACC |
| | | CCUGACCAGGUCGUCGCCAUCGCUUCGAAC |
| | | AUCGGUGGUAAGCAGGCGCUGGAAACUGUC |
| | | CAACGCCUUCUUCCAGUGCUGUGUCAGGAC |
| | | CAUGGGCUUACCCCAGACCAGGUAGUCGCU |
| | | AUAGCAUCAAACGGUGGAGGAAAGCAGGCC |
| | | UUGGAAACGGUGCAGCGGCUCCUGCCAGUC |
| | | CUGUGCCAAGACCACGGCCUGACCCCAGAU |
| | | CAGGUGGUAGCCAUCGCCAGCAAUAACGGU |
| | | GGCAAACAGGCUCUGGAGUCCAUUGUGGCC |
| | | CAGCUGUCGAGGCCUGACCCGGCGCUGGCC |
| | | GCCCUGACCAACGAUCACCUGGUCGCGCUG |
| | | GCCUGUCUCGGAGGCAGACCCGCUAUGGAC |
| | | GCAGUGAAGAAAGGCCUGCCCCAUGCCCCG |
| | | GAACUGAUCCGCAGGGUGAAUCGCCGGAUC |
| | | GGCGAAAGAACCAGCCAUCGAGUGGCCAUC |
| | | UCCCGCGUGGGCGGCUCCUCCAGACGCGAG |
| | | UCCAUAAACCCGUGGAUCCUGACCGGGUUC |
| | | GCCGAUGCCGAAGGUUGCUUCAUGCUGAAU |
| | | AUUUGGAAUAAGAACCGAACUCGGGCCAAA |
| | | UACUACACCGCUCUCCGCUUCGAGAUCGCG |
| | | CUCCACAACAAGGAUAAGUCCAUACUGGAG |
| | | AACAUCCGAUCGACCUGGAAAGUCGGCAAG |
| | | AUCCGCAACAUCGGGGACCGGGUGGUGAAG |
| | | CUGGUCGUGGGACGGUUCGAAGAUUUGAAG |
| | | GUCAUUAUCGACCAUUUCGAGAAGUACCCU |
| | | CUGAUUACGCAGAAGCUGGGGGACUACAAG |
| | | CUGUUCAAACAGGCUUUCUCCCUGAUGGAG |
| | | AACAAGGAACACUUGAAGGAAAACGGCAUC |
| | | AAGGAACUGGUCCGCAUCAAGGCCAAGAUG |
| | | AACUGGGGGCUGAACGACGAGCUGAAGAAG |
| | | GCCUUCCCGGAAAACAUUAGCAAGGAGCGC |
| | | CCGCUCAUCAACAAGAACAUCCCCAACCUG |
| | | AAGUGGCUGGCGGGAUUCACUUCCGGCGAC |
| | | GGUUAUUUUGGAGUGGAACUCAUGAAGAGA |
| | | ACAACCGGAACCCACGUGACCGUGCGACUC |
| | | CGGUUCUCCAUUACCCAGCAUAUCCGGGAC |
| | | AAAAACCUGAUGAACAGCCUGAUUACAUAC |
| | | UUGGGAUGUGGCCGCAUUUCCGAGCGGAAC |
| | | AAGUCCGAAUACAGCUGGCUGGAAUUCGUG |
| | | GUCACCAAGUUCAGCGACAUCAACGACAAG |
| | | AUCAUCCCAGUGUUCCAGGAGAAUACCCUG |
| | | AUUGGAGUGAAGCUCGAGGAUUUUGAGGAC |
| | | UGGUGCAAGGUCGCCAAGCUGAUAGAAGAA |
| | | AAGAAGCAUCUCACCGAAUCAGGCCUGGAU |
| | | GAAAUUAAGAAGAUCAAACUCAACAUGAAU |
| | | AAGGGACGCGUGUUC |
| 10 | Trex2 DNA | ATGTCAGAACCACCTCGCGCTGAGACTTTC |
| | | GTGTTCCTTGACCTTGAAGCCACAGGACTG |
| | | CCCAACATGGACCCGGAAATCGCCGAGATC |
| | | AGCCTCTTCGCGGTGCATCGGTCATCCCTG |
| | | GAGAACCCCGAACGGGACGATTCCGGGTCA |
| | | CTGGTGTTGCCTCGCGTGCTGGATAAGCTG |
| | | ACTCTCTGCATGTGCCCTGAAAGGCCGTTC |
| | | ACCGCCAAAGCGTCGGAAATCACCGGTCTG |
| | | TCGTCCGAATCCCTGATGCATTGCGGAAAG |
| | | GCCGGGTTCAATGGTGCCGTCGTCAGAACC |
| | | CTGCAGGGATTCCTGTCACGGCAGGAAGGT |
| | | CCTATCTGCCTGGTGGCACACAACGGCTTC |
| | | GACTACGACTTTCCCCTCCTGTGTACCGAG |
| | | CTGCAGAGACTCGGAGCCCATCTGCCACAG |
| | | GATACTGTGTGCCTCGACACTCTGCCTGCG |
| | | CTCAGAGGACTTGATCGGGCACACTCCCAT |
| | | GGAACCAGGGCTCAGGGAAGAAAGTCCTAC |
| | | AGCTTGGCCTCGCTGTTCCACCGGTACTTC |
| | | CAGGCAGAACCATCGGCCGCACATTCAGCT |
| | | GAGGGCGATGTGCACACCCTGCTGCTGATC |
| | | TTCCTTCACCGCGCACCTGAACTGCTGGCT |
| | | TGGGCAGACGAACAAGCTAGATCCTGGGCG |
| | | CACATTGAGCCTATGTACGTGCCTCCGGAT |
| | | GGACCTAGCCTCGAGGCCTAG |
| 11 | Trex2 RNA | GGGGCCGCCACCAUGUCAGAACCACCUCGC |
| | | GCUGAGACUUUCGUGUUCCUUGACCUUGAA |
| | | GCCACAGGACUGCCCAACAUGGACCCGGAA |
| | | AUCGCCGAGAUCAGCCUCUUCGCGGUGCAU |
| | | CGGUCAUCCCUGGAGAACCCCGAACGGGAC |
| | | GAUUCCGGGUCACUGGUGUUGCCUCGCGUG |
| | | CUGGAUAAGCUGACUCUCUGCAUGUGCCCU |
| | | GAAAGGCCGUUCACCGCCAAAGCGUCGGAA |
| | | AUCACCGGUCUGUCGUCCGAAUCCCUGAUG |
| | | CAUUGCGGAAAGGCCGGGUUCAAUGGUGCC |
| | | GUCGUCAGAACCCUGCAGGGAUUCCUGUCA |
| | | CGGCAGGAAGGUCCUAUCUGCCUGGUGGCA |
| | | CACAACGGCUUCGACUACGACUUUCCCCUC |
| | | CUGUGUACCGAGCUGCAGAGACUCGGAGCC |
| | | CAUCUGCCACAGGAUACUGUGUGCCUCGAC |
| | | ACUCUGCCUGCGCUCAGAGGACUUGAUCGG |
| | | GCACACUCCCAUGGAACCAGGGCUCAGGGA |
| | | AGAAAGUCCUACAGCUUGGCCUCGCUGUUC |
| | | CACCGGUACUUCCAGGCAGAACCAUCGGCC |
| | | GCACAUUCAGCUGAGGGCGAUGUGCACACC |
| | | CUGCUGCUGAUCUUCCUUCACCGCGCACCU |
| | | GAACUGCUGGCUUGGGCAGACGAACAAGCU |
| | | AGAUCCUGGGCGCACAUUGAGCCUAUGUAC |
| | | GUGCCUCCGGAUGGACCUAGCCUCGAGGCC |
| | | UAGUUAAUUAACGAGCAUCUUACCGCCAUU |
| | | UAUACCGUACGGAAAAAAAAAAAAAAAAAA |
| | | AAAAAAAAAAAAAAAAAAAAAAAAAAAAAA |
| | | AAAAAAAAAAAAAAAAAAAAAAAAAAAAAA |
| | | AAAAAAAAAAAAAAAAAAAAAAAAAAAAAA |
| | | AAAAAAAAAAAAAAAAAAAAAAAAAAAAAA |
| | | AAAAAAAAAAAAAAAAAAAAAAAAAAAAAA |
| | | AAAAAAAAAAAAAAAAAAAAAAAAAAAAAA |
| | | AAAA |
| 12 | Trex2 RNA | AUGUCAGAACCACCUCGCGCUGAGACUUUC |
| | | GUGUUCCUUGACCUUGAAGCCACAGGACUG |
| | | CCCAACAUGGACCCGGAAAUCGCCGAGAUC |
| | | AGCCUCUUCGCGGUGCAUCGGUCAUCCCUG |
| | | GAGAACCCCGAACGGGACGAUUCCGGGUCA |
| | | CUGGUGUUGCCUCGCGUGCUGGAUAAGCUG |
| | | ACUCUCUGCAUGUGCCCUGAAAGGCCGUUC |
| | | ACCGCCAAAGCGUCGGAAAUCACCGGUCUG |
| | | UCGUCCGAAUCCCUGAUGCAUUGCGGAAAG |
| | | GCCGGGUUCAAUGGUGCCGUCGUCAGAACC |
| | | CUGCAGGGAUUCCUGUCACGGCAGGAAGGU |
| | | CCUAUCUGCCUGGUGGCACACAACGGCUUC |
| | | GACUACGACUUUCCCCUCCUGUGUACCGAG |
| | | CUGCAGAGACUCGGAGCCCAUCUGCCACAG |
| | | GAUACUGUGUGCCUCGACACUCUGCCUGCG |
| | | CUCAGAGGACUUGAUCGGGCACACUCCCAU |
| | | GGAACCAGGGCUCAGGGAAGAAAGUCCUAC |
| | | AGCUUGGCCUCGCUGUUCCACCGGUACUUC |
| | | CAGGCAGAACCAUCGGCCGCACAUUCAGCU |
| | | GAGGGCGAUGUGCACACCCUGCUGCUGAUC |
| | | UUCCUUCACCGCGCACCUGAACUGCUGGCU |
| | | UGGGCAGACGAACAAGCUAGAUCCUGGGCG |
| | | CACAUUGAGCCUAUGUACGUGCCUCCGGAU |
| | | GGACCUAGCCUCGAGGCCUAG |

All publications, patent applications, and issued patents cited in this specification are herein incorporated by reference as if each individual publication, patent application, or issued patent were specifically and individually indicated to be incorporated by reference.

Although the foregoing embodiments have been described in some detail by way of illustration and example for purposes of clarity of understanding, it will be readily apparent to one of ordinary skill in the art in light of the teachings contemplated herein that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### A New Method for Removal of dsRNA from Therapeutic RNA Preparations

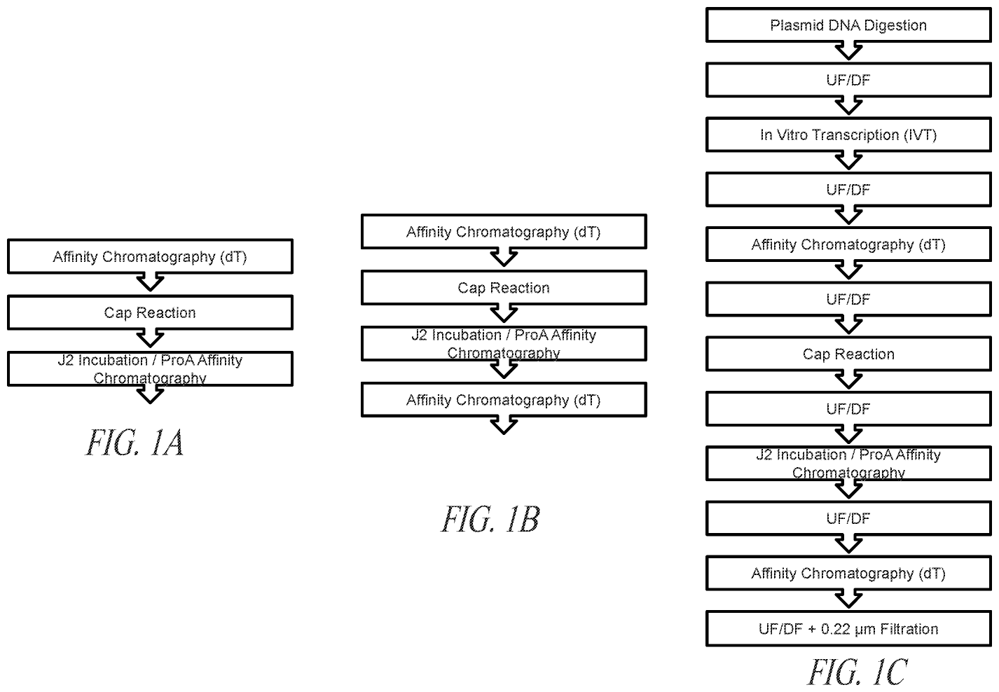

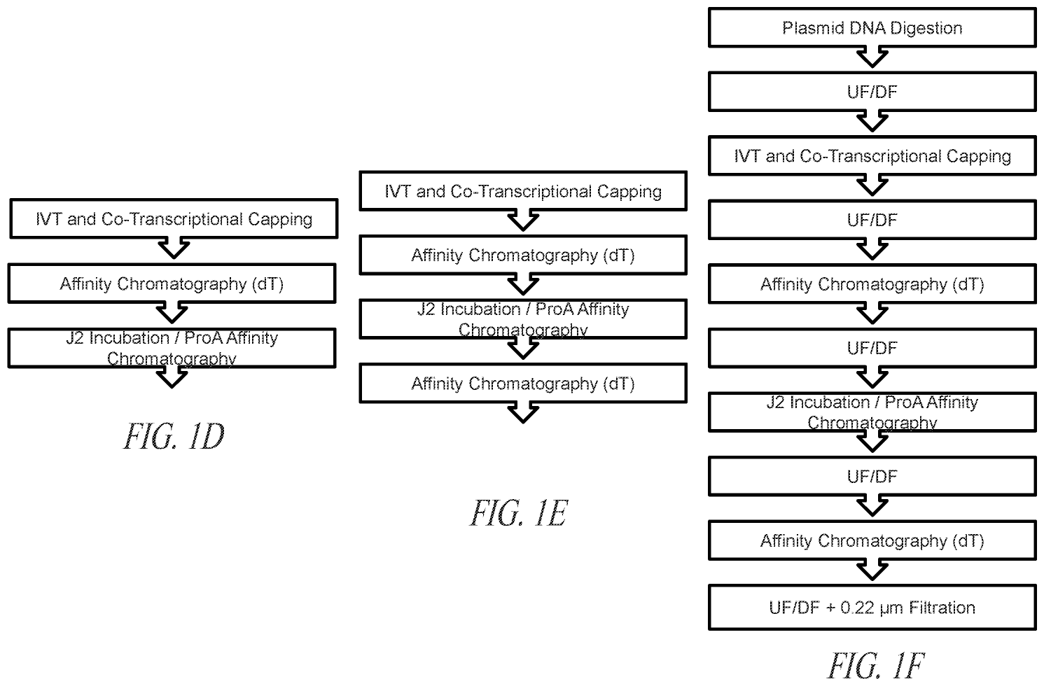

A new and improved method/process for removal of dsRNA was developed which is surprisingly effective and reduces cellular toxicity in vivo. FIGS. 1A-1F show illustrative method unit operations. In brief, the process uses non-amplified, linear DNA (e.g., from a digested plasmid or otherwise) as the template that codes for a gene of interest. An in vitro transcription reaction is used (e.g., using a T7 phage polymerase and nucleotide triphosphates) to synthesize the RNA transcript from the linear DNA. The RNA transcripts are enzymatically capped post translationally (or co-transcriptionally) at the 5′ end using a capping enzyme, e.g., Vaccinia guanylyltransferase, guanosine triphosphate, and S-adenosyl-L-methionine to result in a cap 0 structure. Alternatively, 2′O-methyltransferase can be used to yield a cap 1 structure. The cap 1 structure contains a methylated 2′OH group penultimate nucleotide. In some embodiments, the capping is conducted co-transcriptionally using known methods and/or commercially available products (e.g., CleanCap®).

An antibody that binds dsRNA is then batch incubated with the RNA transcripts to bind to the dsRNA impurities. A resin (e.g., MabCapture™ A Select ProA™ resin) is used to deplete the antibody-dsRNA complex and free antibody from the sample. The RNA transcripts are also chromatographically purified between reaction unit operations by affinity chromatography using oligo dT affinity column/resin (e.g., oligo dT cellulose resin/column or POROS™ Oligo (dT)25 column (SEQ ID NO: 15)), and diafiltrated into the desired formulation buffer. The mRNA is filtrated through a 0.22 μm filter as a final step.

### Example 2

### J2 Antibody Plus Oligo Dt Affinity Chromatography Effectively Removes dsRNA from RNA Preparations

TCRa megaTAL mRNA (SEQ ID NO: 2) was purified using the process unit operations described in Example 1 (see also FIG. 1C). Specifically, the anti-dsRNA antibody J2 bound to MabCapture™ A Select ProA™ resin (ThermoFisher Scientific™) was used to remove dsRNA from the RNA sample. 1 mL and 5 ml columns of ProA resin/J2 antibody were tested (“1× ProA Column” and “5× ProA Column”), as well as use of a 5 ml column (ProA resin/J2 antibody) with an additional step of affinity chromatography purification (oligo dT) after the J2 antibody purification step (“5× ProA Column+dT”). dsRNA content was measured by dsRNA dot blot assay as described in Kariko et al., Nucleic Acids Res. 2011 November; 39(21): e142. In brief, mRNA lots were blotted onto a charged Nytran™ membrane alongside a synthetic 100% double stranded RNA control. The membrane was dried, blocked, and incubated with J2 anti-dsRNA IgG2a monoclonal antibody overnight. After several washes, a fluorescent secondary antibody is then used to bind to the J2 antibody. After several washes, images were captured using a LI-COR Odyssey CLx Imaging System and analyses of percent double stranded RNA in mRNA lots were performed by fluorescent intensity comparison to the 100% control.

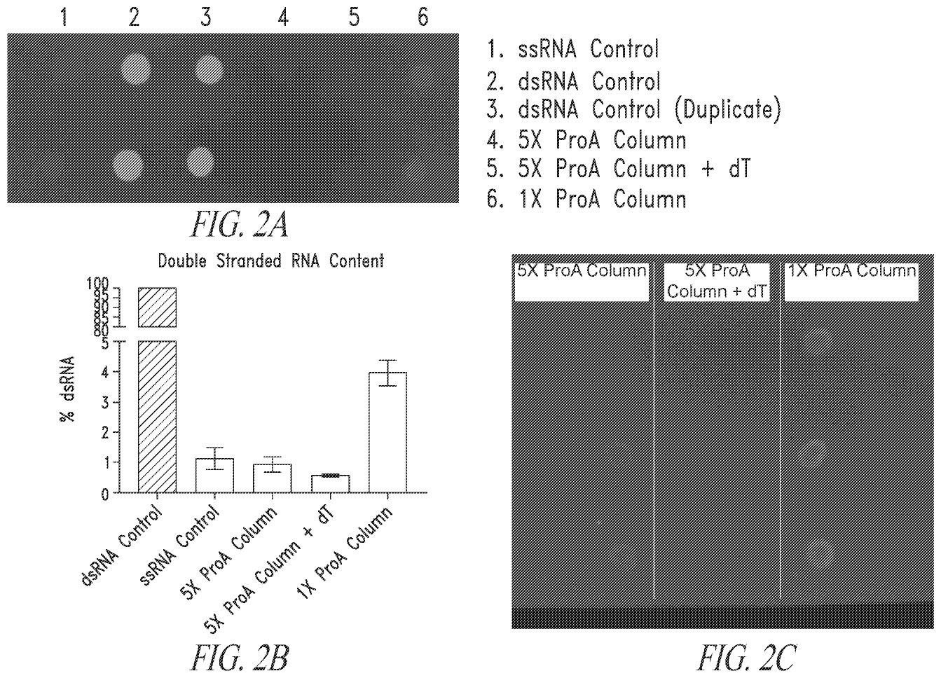

With a 1 mL column, there was notable depletion of dsRNA content as indicated by reduced fluorescence (FIGS. 2A and 2B). Further depletion of antibody:dsRNA complexes was observed by increasing the volume of ProA resin, i.e., by using a 5 ml column. However, surprisingly, further depletion of antibody:dsRNA complexes was achieved by adding an affinity chromatography purification step (i.e., an oligo dT purification step) after the J2 antibody purification step (FIG. 2B). Moreover, a direct dot blot of samples with fluorescently-labeled secondary indicates that residual fluorescence signals detected the FIG. 2B dot blot were due to residual J2 antibodies which passed through the ProA column (FIG. 2C). Accordingly, evaluation of dsRNA content by dsRNA dot blot shows that the percentage of dsRNA in the mRNA sample is undetectable by the assay when combining antibody and oligo dT based purifications as described above

### Example 3

### J2 Antibody Titration

J2 titration experiments to determine the effective quantity of J2 mAb for double-stranded RNA (dsRNA) clearance was performed. PD1 megaTAL (SEQ ID NO: 5; ˜3000 nt) and Trex2 (SEQ ID NO: 11; ˜1000 nt) mRNAs were prepared using an in-house mRNA production process. In brief, the mRNA materials were generated by in vitro transcription and capped at the 5′-end with a cap 0 structure prior to the J2 titration experiments. Quantities of J2 were calculated according to the mRNA molecule (up to 60 mol %). Samples were incubated with the appropriate quantities of J2 at room temperature for 30 min followed by the addition of ProA resin to capture the antibody:dsRNA complex (incubation at room temperature for 1 hour). The purified mRNA materials were then collected through a vacuum manifold. J2 dot blot (described above) and impedance assay (described below) were performed to determine the dsRNA content and toxicity to BJ fibroblasts cells.

Evaluations of cytotoxicity of mRNA lots were performed via a cellular impedance assay using BJ Fibroblast cells and the xCELLigence™ RTCA MP Instrument from ACEA Biosciences, Inc. The xCELLigence instrument uses noninvasive electrical impedance monitoring to continuously measure cell viability in the form of a “Cell Index” value. Cells were adhered to ACEA's E-plates containing interdigitated electrodes and given 24 hours to proliferate. Cells were then transfected with mRNA lots and a double stranded mRNA killing control and monitored for 72 hours post-transfection. The ACEA software is used to analyze the cell index value for each well over the 72-hour window post-transfection and report a value for the slope of the cell index. The slope of cell index of a given mRNA lot is compared to the slopes of cell index of the LNP only and double stranded killing controls to give an indication of cytotoxicity.

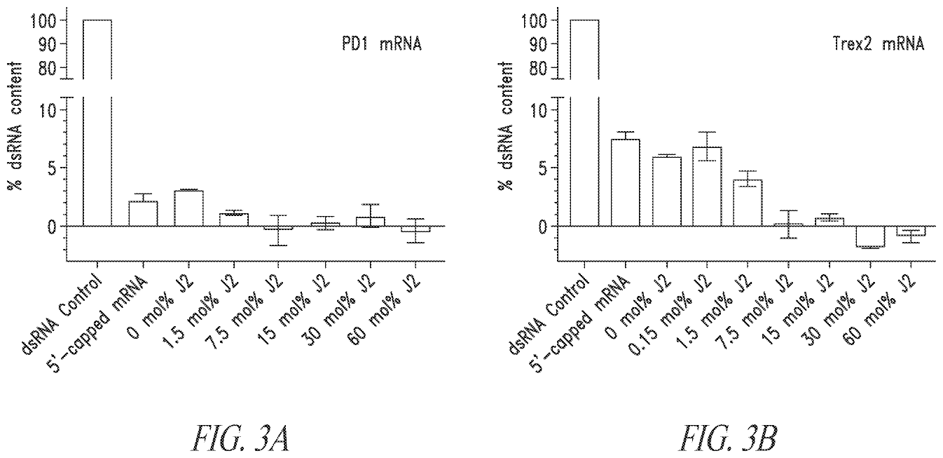

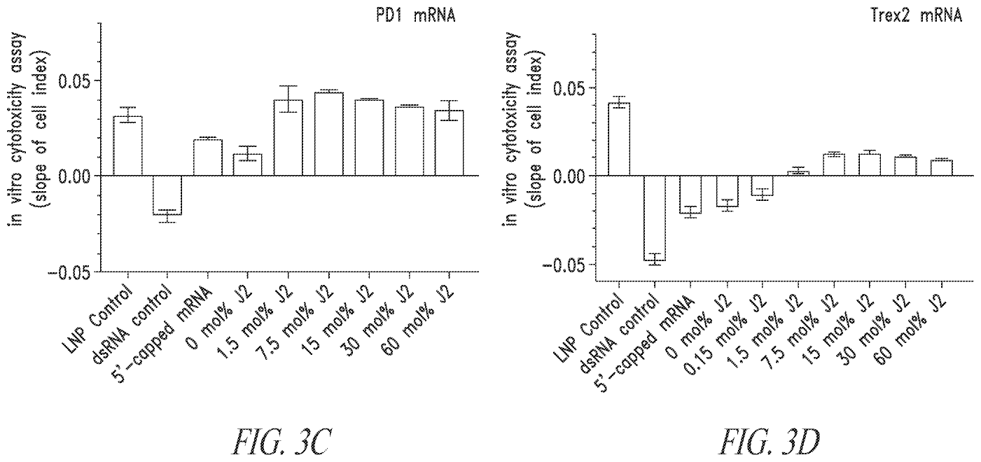

The titration experiments with both mRNA constructs indicated effective dsRNA clearance at ≥7.5 mol % J2 (FIGS. 3A and 3B). In addition, in vitro toxicity result correlates well with dsRNA content, showing reduced cytotoxicity at lower dsRNA level (FIGS. 3C and 3D).

### Example 4

### In Vivo Gene Editing and mRNA Characteristics

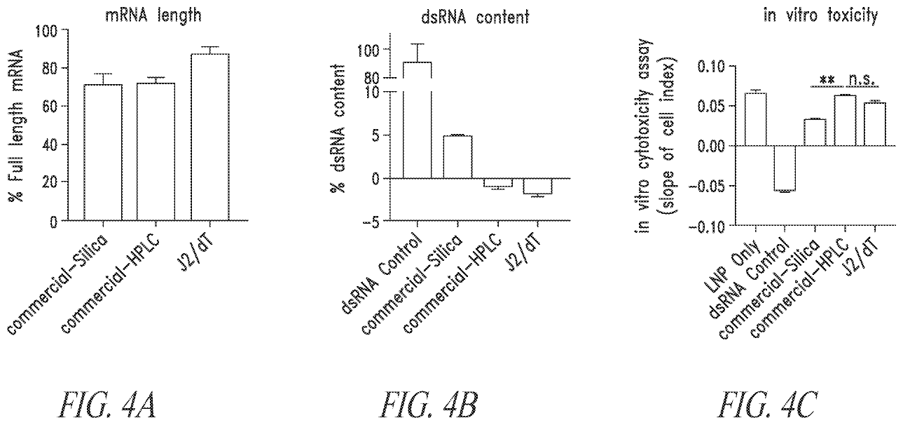

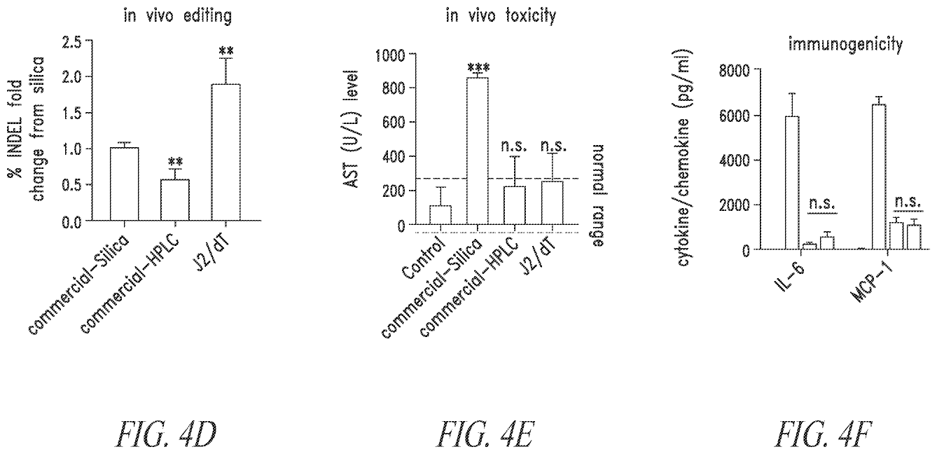

In-house generated PCSK9 megaTAL and Trex2 mRNAs (SEQ ID NOs: 8 and 11, respectively) crude in vitro transcription (IVT) RNA material was sent to a commercial vendor to be capped and purified with either a silica resin (commercial—Silica) or HPLC (commercial—HPLC). The same crude IVT material was also purified in-house with a poly(A) mRNA isolation (oligo dT purification) and dsRNA depletion (J2 purification), as described above. PCSK9 megaTAL mRNA purified using the three methods were compared in three separate in vitro assays (FIGS. 4A-4F). mRNA length was measured by running the mRNA on an Advanced Analytical, capillary electrophoreses based Fragment Analyzer using their standard RNA analysis reagents per the manufacturer's recommended protocol. The area under the curve was measured using ProSize software (Agilent Technologies, Inc) and the average total percent area of the selected peak for three replicates was plotted (FIG. 4A).

Double-stranded mRNA (dsRNA) can be toxic when delivered in vivo. To measure the amount of dsRNA in the mRNA preparations, dsRNA dot-blot assay was performed as described above. J2/dT mRNA production process produces mRNA with undetectable levels of dsRNA, similar to, or better than, the HPLC and silica purified mRNA (FIG. 4B).

mRNA toxicity was measured in an in vitro cell growth assay using ACEA Biosciences, Inc.'s RTCA iCELLigence™ impedance-based assy. Human BJ fibroblasts (ATCC, CRL-2522) cells were seeded into the iCELLigence plate and allowed to adhere for 18-24 hours. mRNA was formulated into Lipofectamine MessengerMax transfection reagent per the manufacturer's recommend protocol and used to transfect the cells. The amount of cell growth was measured for 48 hours as and the slope of growth is graphed as an indicator of toxicity (FIG. 4C).

PCSK9 megaTAL and Trex2 mRNA purified using the three methods were formulated with liquid nano particles (LNPs) (Acuitas Therapeutics) in a 1.0:1.0 molar ratio. mRNA/LNP formulations where diluted in phosphate buffered saline (PBS) and administered (via tail vein injection) to five Balb/C mice per condition at a dose of 1 mg/kg (FIGS. 4D-4F). INDEL analysis was performed using next-generation amplicon sequencing and graphed as fold change compared to the silica condition (FIG. 4D).

The relative toxicity of each mRNA preparation was assayed by taking mandibular bleeds from animals 24 hours after dosing and measuring Aspartate Aminotransferase (AST) enzyme levels (FIG. 4E). The in vivo immunogenicity of the purified mRNAs was measured by quantifying chemokine and cytokine levels (i.e., IL-6 and MCP-1) from serum collected four hours after mRNA formulation dosing using EMD Millipore's MILLIPLEX MAP Mouse Cytokine/Chemokine Magnetic Bead based Luminex panel (cat #: MCYTOMAG-70KM) (FIG. 4F).

Overall, the in-house (J2/dT) method comprising poly(A) mRNA purification and dsRNA depletion produced mRNA that was the same quality or better quality than the commercially sourced silica or HPLC purified mRNA, in both in vitro mRNA characteristics or in in vivo activity and toxicity/immunogenicity assays.

### Example 5

### Ex Vivo Gene Editing and mRNA Characteristics

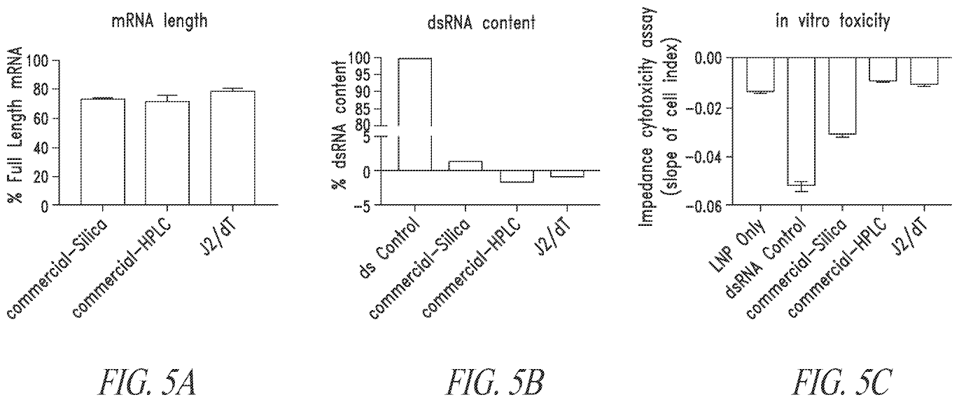

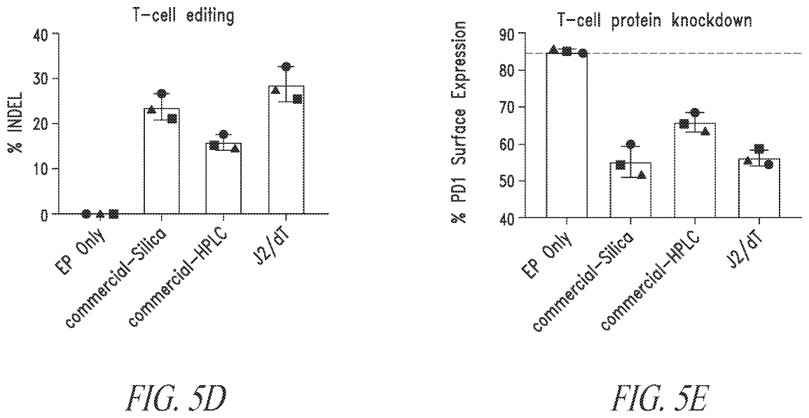

In-house generated PD1 megaTAL mRNA (SEQ ID NO: 5) crude in vitro transcription (IVT) RNA material was sent to a commercial vendor to be capped and purified with either a silica resin (commercial—Silica) or HPLC (commercial—HPLC). The same crude IVT material was also purified in-house with a poly(A) mRNA isolation (oligo dT purification) and dsRNA depletion (J2 purification). mRNA made using the three methods were compared in three separate assays to assess mRNA quality (FIG. 5A-5C). The mRNA was also evaluated in T-cells to compare differences in efficacy (FIGS. 5D and 5E). PBMC's from three donors were stimulated with αCD3 and αCD28 antibodies. After 72 hours in 37° C., T-cells were electroporated with mRNA using the Amaxa 4D-Nucleofector at a 50 μg/mL dose. Each mRNA was electroporated in triplicate for each of the three donors. After electroporation, cells were put in 30° C. for overnight recovery and then moved to 37° C. the next day. 96 hours after electroporation cells were split into two plates. One plate was stimulated with PMA/ionomycin for 24 hours and then analyzed of FACS to observe PD-1 knockdown in PD1 megaTAL mRNA treated cells (FIG. 5E). The remaining plate was processed for INDEL analysis via NGS (FIG. 5D).

### Example 6

### Comparison of dsRNA Levels Between Different Purification Methods

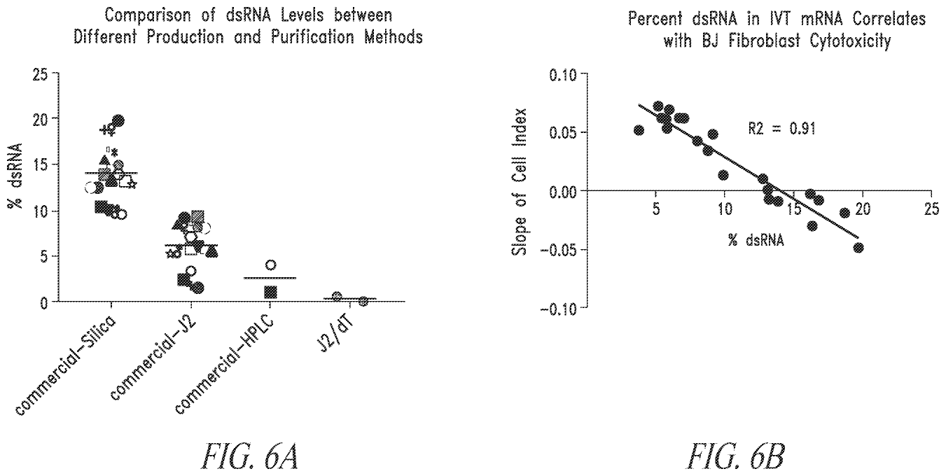

dsRNA levels were compared among mRNAs generated by different purification methods (FIG. 6A). Multiple mRNA constructs were made by a commercial supplier using their platform silica or HPLC purification process (commercial-Silica or commercial-HPLC). In addition, a portion of each silica-purified mRNA was further purified using bluebird's J2/dT process (commercial-J2). Two batches of in-house-generated mRNA using J2/dT process were included in the comparison. The analysis demonstrated that the additional J2/dT purification surprisingly decreased the dsRNA levels of silica-purified materials. Furthermore, in-house-generated materials purified by J2/dT exhibited the lowest dsRNA levels.

In addition to dsRNA analysis by dot blot, in vitro cytotoxicity was evaluated for a selective group of mRNA materials by an impedance-based assay using BJ fibroblast cells. The cytotoxicity results, which were expressed as the slopes of cell growth index, showed strong correlation with the dsRNA levels of the mRNA materials (FIG. 6B).

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. An RNA purification process comprising:
(a) contacting an RNA sample comprising single-stranded RNA and double-stranded RNA (dsRNA) with an antibody or antigen binding fragment thereof that binds dsRNA, thus forming dsRNA:antibody complexes;
(b) removing the dsRNA:antibody complexes from the sample; and
(c) purifying the single-stranded RNA.

2. A process for producing therapeutic RNA comprising:
(a) contacting an RNA sample comprising single-stranded RNA and double-stranded RNA (dsRNA) with an antibody or antigen binding fragment thereof that binds dsRNA, thus forming dsRNA:antibody complexes;
(b) removing the dsRNA:antibody complexes from the sample; and
(c) purifying the single-stranded RNA;
thereby producing therapeutic RNA.

3. A process for increasing nuclease editing efficiency comprising:
(a) contacting an RNA sample comprising single-stranded RNA and double-stranded RNA (dsRNA) encoding a nuclease with an antibody or antigen binding fragment thereof that binds dsRNA, thus forming dsRNA:antibody complexes;
(b) removing the dsRNA:antibody complexes from the sample; and
(c) purifying the single-stranded RNA;
wherein the editing rate of the nuclease is increased compared to the editing rate of a nuclease encoded by an RNA that is not contacted with an antibody that binds dsRNA.

4. A process for decreasing the immunogenicity and/or toxicity of RNA administered to a cell or subject comprising:
(a) contacting an RNA sample comprising single-stranded RNA and double-stranded RNA (dsRNA) with an antibody or antigen binding fragment thereof that binds dsRNA, thus forming dsRNA:antibody complexes;
(b) removing the dsRNA:antibody complexes from the sample; and
(c) purifying the single-stranded RNA;
wherein the immunogenicity and/or toxicity of the RNA when administered to a cell or subject is less than the immunogenicity and/or toxicity of an RNA administered to a cell or subject when the RNA has not been contacted with an antibody that binds dsRNA.

5. The process of claim 4, wherein the subject is human.

6. The process of any one of claims 1-5, wherein the single-stranded RNA is single-stranded circular RNA, single-stranded mRNA, or single-stranded non-coding RNA.

7. The process of any one of claims 1-6, wherein the single-stranded RNA is polyadenylated and/or the process comprises a polyadenylation step prior to contacting the sample with an antibody or antigen binding fragment thereof that binds dsRNA.

8. The process of claim 7, wherein the process comprises contacting the polyadenylated RNA sample with a first oligonucleotide dT (oligo dT) probe that binds polyadenylated RNA and removing unbound RNA from the sample prior to contacting the sample with an antibody or antigen binding fragment thereof that binds dsRNA.

9. The process of claim 7 or claim 8, wherein the process further comprises contacting the polyadenylated RNA with a second oligonucleotide dT probe after contacting with the antibody or antigen binding fragment thereof that binds dsRNA.

10. The process of any one of claims 1-9, wherein the RNA sample is obtained de novo through chemical synthesis.

11. The process of any one of claims 1-9, wherein the RNA sample is obtained from an in vitro transcription reaction.

12. The process of any one of claims 1-11, wherein cytotoxicity, as measured by impedance, of the purified RNA when administered to a cell is less than the cytotoxicity of RNA administered to a cell, when the RNA has not been contacted with an antibody that binds dsRNA and/or a second oligo dT.

13. The process of any one of claims 8-12, wherein the first and/or second oligonucleotide dT probe is bound to a surface.

14. The process of claim 13, wherein the first and/or second oligonucleotide dT probe is covalently linked to the surface.

15. The process of any one of claims 1-14, wherein the RNA in the sample is capped and/or the process comprises capping the RNA in the sample.

16. The process of any one of claims 1-15, wherein the RNA is obtained from an in vitro transcription reaction and is co-transcriptionally capped.

17. The process of claim 15 or claim 16, wherein the cap is a cap0 or cap1.

18. The process of claim 15 or claim 16, wherein the cap is an ARCA cap or modified ARCA cap.

19. The process of any one of claims 1-18, wherein the RNA in the sample is capped at its 5′ end using a capping enzyme, guanosine triphosphate, and S-adenosyl-L-methionine.

20. The process of claim 19, wherein the capping enzyme is Vaccinia guanylyltransferase.

21. The process of claim 19 or claim 20, wherein the capping comprises guanosine triphosphate.

22. The process of any one of claim 15-21, wherein the capping comprises S-adenosyl-L-methionine.

23. The process of any one of claims 15-22, wherein the capping comprises a 2′-O-Methyltransferase.

24. The process of any one of claims 1-23, wherein the antibody or antigen binding fragment thereof that binds dsRNA is selected from the group consisting of: a Camel Ig, a Llama Ig, an Alpaca Ig, Ig NAR, a Fab′ fragment, a F(ab′)2 fragment, a bispecific Fab dimer (Fab2), a trispecific Fab trimer (Fab3), an Fv, an single chain Fv protein (“scFv”), a bis-scFv, (scFv)2, a minibody, a diabody, a triabody, a tetrabody, a disulfide stabilized Fv protein (“dsFv”), and a single-domain antibody (sdAb, a camelid VHH, Nanobody).

25. The process of any one of claims 1-24, wherein the antibody or antigen binding fragment thereof that binds dsRNA is a monoclonal antibody.

26. The process of any one of claims 1-25, wherein the antibody is selected from the group consisting of: J2, J5, K1, K2, 1D3, CABT-B212, and 9D5.

27. The process of any one of claims 1-26, wherein the antibody is J2.

28. The process of any one of claims 1-27, wherein the RNA is contacted with at least about 1.5 mol %, at about least 2 mol %, at least about 2.5 mol %, at least about 3 mol %, at least about 3.5 mol %, at least about 4 mol %, at least about 4.5 mol %, at least about 5 mol %, at least about 5.5 mol %, at least about 6 mol %, at least about 6.5 mol %, at least about 7 mol %, at least about 7.5 mol %, at least about 15 mol %, at least about 30 mol %, or at least about 60 mol % antibody, compared to total moles of RNA within the sample.

29. The process of any one of claims 1-27, wherein the RNA is contacted with at least about 1.5 mol %, at least about 7.5 mol %, at least about 15 mol %, at least about 30 mol %, or at least about 60 mol % antibody, compared to total moles of RNA within the sample.

30. The process of any one of claims 1-27, wherein the sample is contacted with at least about 7.5 mol % antibody, compared to total moles of RNA within the sample.

31. The process of any one of claims 1-27, wherein the sample is contacted with about 1.5 mol %, about 2 mol %, about 2.5 mol %, about 3 mol %, about 3.5 mol %, about 4 mol %, about 4.5 mol %, about 5 mol %, about 5.5 mol %, about 6 mol %, about 6.5 mol %, about 7 mol %, about 7.5 mol %, about 15 mol %, about 30 mol %, or about 60 mol % antibody, compared to total moles of RNA within the sample.

32. The process of any one of claims 1-27, wherein the sample is contacted with about 7.5 mol % antibody, compared to total moles of RNA within the sample.

33. The process of any one of claims 1-27, wherein the sample is contacted with about 1.5 mol % to about 60 mol % antibody, compared to total moles of RNA within the sample.

34. The process of any one of claim 1-27, wherein the sample is contacted with about 2 mol % to about 60 mol % antibody, compared to total moles of RNA within the sample.

35. The process of any one of claims 1-27, wherein the sample is contacted with about 2.5 mol % to about 60 mol % antibody, compared to total moles of RNA within the sample.

36. The process of any one of claims 1-27, wherein the sample is contacted with about 3 mol % to about 60 mol % antibody, compared to total moles of RNA within the sample.

37. The process of any one of claims 1-27, wherein the sample is contacted with about 3.5 mol % to about 60 mol % antibody, compared to total moles of RNA within the sample.

38. The process of any one of claims 1-27, wherein the sample is contacted with about 4 mol % to about 60 mol % antibody, compared to total moles of RNA within the sample.

39. The process of any one of claims 1-27, wherein the sample is contacted with about 4.5 mol % to about 60 mol % antibody, compared to total moles of RNA within the sample.

40. The process of any one of claims 1-27, wherein the sample is contacted with about 5 mol % to about 60 mol % antibody, compared to total moles of RNA within the sample.

41. The process of any one of claims 1-27, wherein the sample is contacted with about 5.5 mol % to about 60 mol % antibody, compared to total moles of RNA within the sample.

42. The process of any one of claims 1-27, wherein the sample is contacted with about 6 mol % to about 60 mol % antibody, compared to total moles of RNA within the sample.

43. The process of any one of claims 1-27, wherein the sample is contacted with about 6.5 mol % to about 60 mol % antibody, compared to total moles of RNA within the sample.

44. The process of any one of claims 1-27, wherein the sample is contacted with about 7 mol % to about 60 mol % antibody, compared to total moles of RNA within the sample.

45. The process of any one of claims 1-27, wherein the sample is contacted with about 7.5 mol % to about 60 mol % antibody, compared to total moles of RNA within the sample.

46. The process of any one of claims 1-27, wherein the sample is contacted with about 15 mol % to about 60 mol % antibody, compared to total moles of RNA within the sample.

47. The process of any one of claims 1-27, wherein the sample is contacted with about 30 mol % to about 60 mol % antibody, compared to total moles of RNA within the sample.

48. The process of any one of claims 1-27, wherein the sample is contacted with about 1.5 mol % to about 30 mol % antibody, compared to total moles of RNA within the sample.

49. The process of any one of claims 1-27, wherein the sample is contacted with about 1.5 mol % to about 15 mol % antibody, compared to total moles of RNA within the sample.

50. The process of any one of claims 1-27, wherein the sample is contacted with about 1.5 mol % to about 7.5 mol % antibody, compared to total moles of RNA within the sample.

51. The process of any one of claims 1-27, wherein the sample is contacted with about 1.5 mol % to about 7 mol % antibody, compared to total moles of RNA within the sample.

52. The process of any one of claims 1-27, wherein the sample is contacted with about 1.5 mol % to about 6.5 mol % antibody, compared to total moles of RNA within the sample.

53. The process of any one of claims 1-27, wherein the sample is contacted with about 1.5 mol % to about 6 mol % antibody, compared to total moles of RNA within the sample.

54. The process of any one of claims 1-27, wherein the sample is contacted with about 1.5 mol % to about 5.5 mol % antibody, compared to total moles of RNA within the sample.

55. The process of any one of claims 1-27, wherein the sample is contacted with about 1.5 mol % to about 5 mol % antibody, compared to total moles of RNA within the sample.

56. The process of any one of claims 1-27, wherein the sample is contacted with about 1.5 mol % to about 4.5 mol % antibody, compared to total moles of RNA within the sample.

57. The process of any one of claims 1-27, wherein the sample is contacted with about 1.5 mol % to about 4 mol % antibody, compared to total moles of RNA within the sample.

58. The process of any one of claims 1-27, wherein the sample is contacted with about 1.5 mol % to about 3.5 mol % antibody, compared to total moles of RNA within the sample.

59. The process of any one of claims 1-27, wherein the sample is contacted with about 1.5 mol % to about 3 mol % antibody, compared to total moles of RNA within the sample.

60. The process of any one of claims 1-27, wherein the sample is contacted with about 1.5 mol % to about 2.5 mol % antibody, compared to total moles of RNA within the sample.

61. The process of any one of claims 1-27, wherein the sample is contacted with about 1.5 mol % to about 2 mol % antibody, compared to total moles of RNA within the sample.

62. The process of any one of claims 1 to 61, wherein the dsRNA:antibody complex is separated from the single-stranded RNA by antibody-based affinity chromatography.

63. The process of claim 62, wherein the antibody-based affinity chromatography comprises a 1 ml column.

64. The process of claim 62, wherein the antibody-based affinity chromatography comprises a 5 ml column.

65. The process of claim 62, wherein the antibody-based affinity chromatography comprises a 10 ml column.

66. The process of any one of the preceding claims, wherein the process comprises a plasmid digestion step prior to an IVT step.

67. The process of any one of the preceding claims, wherein the process further comprises a step of treating the sample with a DNase to remove residual plasmid DNA template.

68. The process of claim 67, wherein the DNase treatment step occurs after an IVT step and/or after a capping step.

69. The process of any one of the preceding claims, further comprising one or more ultrafiltration/diafiltration steps.

70. The process of claim 69, wherein the UF/DF step is after a plasmid digestion step, an in vitro transcription step, a cap reaction step, or an affinity chromatography step (e.g., dT or J2).

71. The process of any one the preceding claims, further comprising a final sterile filtration step.

72. The process of claim 71, wherein the final sterile filtration step comprises filtration through a 0.22 μm filter.

73. The process of any one of claims 3 and 6-72, wherein the nuclease is an endonuclease or exonuclease.

74. The process of any one of claims 3 and 6-73, wherein the nuclease is a homing endonuclease, megaTAL, CRISPR-associated nuclease, zinc finger nuclease, transcription activator-like effector nuclease (TALEN).

75. The process of claim 74, wherein the CRISPR-associated nuclease is Cas9 or a variant thereof.

76. The process of any one of claims 1-75, wherein Aspartate Aminotransferase enzyme (AST) levels in a subject administered the purified RNA are less than AST levels in a subject administered purified RNA not contacted with an antibody that binds dsRNA and/or a second oligo dT.

77. The process of any one of claims 1-76, wherein IL-6 levels in a subject administered the purified RNA are less than IL-6 levels in a subject administered purified RNA not contacted with an antibody that binds dsRNA and/or a second oligo dT.

78. The process of any one of claims 1-77, wherein MCP-1 levels in a subject administered the purified RNA are less than MCP-1 levels in a subject administered purified RNA not contacted with an antibody that binds dsRNA and/or a second oligo dT.
